(19) United States
(12) Patent Application Publication
(10) Pub. No.: US 2025/0020645 A1
(43) Pub. Date: Jan. 16, 2025

(54) A4B1 BINDING APTAMERS

(71) Applicants: University of Washington, Seattle, WA (US); Seattle Children's Hospital d/b/a Seattle Children's Research Institute, Seattle, WA (US)

(72) Inventors: Suzie Hwang Pun, Seattle, WA (US); Ian Cardle, Seattle, WA (US); Ian Cardle, Seattle, WA (US); Michael C. Jensen, Bainbridge Island, WA (US); Dinh Chuong Nguyen, Seattle, WA (US); Yuan-Che Wu, Seattle, WA (US)

(73) Assignees: University of Washington, Seattle, WA (US); Seattle Children's Hospital d/b/a Seattle Children's Research Institute, Seattle, WA (US)

(21) Appl. No.: 18/771,737

(22) Filed: Jul. 12, 2024

Related U.S. Application Data
(60) Provisional application No. 63/513,334, filed on Jul. 12, 2023.

(51) Int. Cl. G01N 33/569; C12N 15/115; G01N 33/53

(52) U.S. Cl. CPC G01N 33/56966; C12N 15/115; G01N 33/5308; C12N 2310/16; C12N 2310/313; C12N 2310/531; G01N 2333/7055

(57) ABSTRACT

Various implementations described herein relate to oligonucleotides that specifically bind α4β1. According to some implementations, oligonucleotides are conjugated to a support, a tag, a linker, or a drug. Compositions described herein can be used for diagnosis or treatment of various diseases, such as T cell-mediated autoimmune diseases. An example method includes exposing a solution of cells to the oligonucleotides and isolating cells that express α4β1 from the solution of cells, wherein the cells that express α4β1 are bound to the oligonucleotides. Example methods and compositions described herein can be used for cell selection, diagnostic, therapeutic, or research purposes.

### CROSS-REFERENCE TO RELATED APPLICATION

This application claims priority to U.S. Provisional Patent Application No. 63/513,334 filed on Jul. 12, 2023, which is incorporated herein by reference in its entirety as if fully set forth herein.

### REFERENCE TO SEQUENCE LISTING

The Sequence Listing associated with this application is provided in XML format in lieu of a paper copy and is hereby incorporated by reference into the specification. The name of the file containing the Sequence Listing is Seq_W149-0046US2.xml. The file is 79,775 bytes, was created Jul. 10, 2024, and is being submitted electronically via Patent Center.

### TECHNICAL FIELD

This application relates to oligonucleotides that specifically bind α4β1, as well as uses thereof.

### BACKGROUND

Lymphomas and lymphocytic leukemias are estimated to account for 6.0% and 4.5% of new cancer cases and deaths, respectively, in the United States in 2022, and children and young adults are disproportionately afflicted with these cancers (Siegel et al., (2024) CA Cancer J Clin, 74, 12-49; Miller et al., (2020) CA Cancer J Clin, 70, 443-459). While the overall survival rate of children and young adults diagnosed with these conditions has improved dramatically in recent decades, approximately 10-20% of them will suffer from progressive disease or relapse (Hunger et al., (2012) J Clin Oncol, 30, 1663-9; Burkhardt et al., (2009) J Clin Oncol, 27, 3363-9; Schrappe et al., (2012) New England Journal of Medicine, 366, 1371-81; Bhojwani and Pui. (2013) Lancet Oncol, 14, e205-e217; Sellar et al., (2018) Br J Haematol, 181, 515-522; Quist-Paulsen et al., (2020) Leukemia, 34, 347-357). Of these patients with relapsed or refractory (r/r) disease, only 15-50% will survive, with especially poor outcomes associated with lymphomas and leukemias derived from T cells (Burkhardt, supra; Schrappe, supra, Sellar, supra; Quist-Paulsen, supra; Nguyen et al., (2008) Leukemia, 22, 2142-50; Ko et al., (2010) J Clin Oncol, 28, 648-54; Trausti et al., (2016) Haematologica, 101, 68-76; Sun et al. (2018) Leukemia, 32, 2316-25; Karrman and Johansson. (2017) Genes Chromosomes Cancer, 56, 89-116). Treatment of newly diagnosed T-cell malignancies usually involves multiagent, escalating chemotherapy, and curative allogeneic hematopoietic stem cell transplantation is only used during successful remission reinduction following disease recurrence, which is achieved in only 30-40% cases of relapsed disease (Raetz and Teachey. (2016) Hematology, 2016, 580-588). Accordingly, new therapeutic strategies that can achieve durable responses in these subsets of patients are urgently required.

While targeted immunotherapy has taken center stage in recent years as a revolutionary cancer treatment for B-cell malignancies, these treatments have remained largely ineffective for T-cell malignancies due to challenges in finding markers selectively expressed on malignant cells over healthy T cells (Scherer et al., (2019) Front Oncol, 9, 126; Alcantara et al., (2018) Leukemia, 32, 2307-15). Unlike B-cell aplasia and hypogammaglobulinemia than can be tolerated with periodic prophylactic immunoglobulin replacement after immunotherapy targeting of B-cell malignancies, prolonged T-cell aplasia as a result of non-selective therapeutic targeting of T-cell malignancies is a nontrivial toxicity with fatal consequences (Wayner et al., (1989) Journal of Cell Biology, 109, 1321-30). The discovery of biomarkers and associated targeting reagents that can sufficiently distinguish T-leukemia and lymphoma cells from healthy counterparts would thus be of high value for designing effective treatments for these diseases.

Designing targeted therapies for T-cell leukemia and lymphoma has been a challenging task. Due to the shared lineage between healthy and malignant T cells, biomarkers that can be uniquely or preferentially targeted for an anti-cancer therapy are limited (Chiaretti et al., (2014) Mediterr J Hematol Infect Dis, 6; Patel et al., (2012) Br J Haematol, 159, 454-61). Therapeutic targeting of pan T-cell markers such as CD2, CD3, CD5, and CD7 carries the risk of T-cell aplasia, which would result in severe immunodeficiency that exposes patients to fatal opportunistic infections (Leonard, W. J. (2001) Nat Rev Immunol, 1, 200-8). More restricted T-cell antigens, including terminal deoxynucleotidyl transferase (TdT), T cell receptor beta constant 1 (TRBC1), CD1a, and CD30, are either expressed intracellularly or only on a subset of T-cell malignancies, hampering their broader targeting appeal (Patel, supra; Scherer, supra; Ali et al., (2022) Nat Biotechnol, 40, 488-98). For these reasons, the identification of antigens selectively and uniformly expressed on T-cell malignancies and the development of associated targeting ligands remain important goals for treating these cancers.

Monoclonal antibodies have been widely used at targeting ligands for theranostic applications, owing to their high specificities and nanomolar binding affinities. However, antibodies are expensive for oncology use due to their biological production (>$100,000 annually), and their large size (150 kDa) prohibits deep tissue penetration needed for comprehensive tumor targeting (Hernandez et al., (2018) Am J Manag Care, 24, 109-12; Thurber et al., (2008) Adv Drug Deliv Rev, 60, 1421-34). Additionally, antibodies are recycled via neonatal Fc receptor binding, leading to long circulation half-lives in patients (several days to weeks) that can prevent rapid management of treatment-associated side effects (Mankarious et al., (1988) J Lab Clin Med, 112, 634-40; Mackness et al. (2019) MAbs, 11, 1276-88). For treating T-cell malignancies, more short-lived targeting approaches may be preferred to prevent prolonged T-cell aplasia and preferentially target antigens that are upregulated on malignant cells but not strictly tumor-specific (Alcantara, supra).

Considered nucleic acid analogues of monoclonal antibodies, aptamers fulfill many of the unique needs for safe and effective targeting of T-cell malignancies. Aptamers are single-stranded oligonucleotides that fold into sequence-specific structures capable of recognizing almost any kind of target with high affinity and specificity. The small size of aptamers (6-30 kDa) allows them to possess higher tumor penetration and shorter circulation half-lives than antibodies, simplifying the management of their concentrations in vivo (Xiang et al., (2015) Theranostics, 5, 1083-97; Healy et al., (2004) Pharm Res, 21, 2234-46). Aptamers are also synthetic, making them inexpensive to manufacture and permissible to many chemical modifications for diverse functions (Zhou and Rossi. (2017) Nat Rev Drug Discov, 16, 440). Given these favorable properties, aptamers have garnered considerable interest as targeting ligands for cancer theranostic applications (Shigdar et al., (2021) Molecular Therapy, 29, 2396-2411; Bohrmann et al., (2022) Theranostics, 12, 4010-50).

### BRIEF DESCRIPTION OF THE DRAWINGS

FIG. 1 illustrates an example environment 100 for using an oligonucleotide 102 that specifically binds α4β1.

FIGS. 2A-2C illustrate that HR7A1 binds α4β1 with single-digit nanomolar affinity and can be significantly truncated without loss of function.

FIGS. 3A, 3B illustrate that aptamer pools from rounds 4-7 of cell-SELEX non-selectively bind both H9 parental and H9 4IgB7H3 cells.

FIGS. 4A-C illustrate the identification of HR7A1, a highly enriched aptamer that broadly binds lymphoma and lymphocytic leukemia cells.

FIGS. 5A, 5B illustrate that integrin α4β1 is identified as a potential target of HR7A1.

FIGS. 6A-6C illustrate that HR7A1 interacts with both CD49d (α4) and CD29 (β1) for selective recognition of α4β1.

FIG. 7 illustrates that CD49d and CD29 expression on α4+β1+ cell lines correlate with each other.

FIGS. 8A, 8B illustrate that HR7A1.Tr3 does not retain binding capabilities of full-length aptamer.

FIGS. 9A, 9B illustrate that HR7A1.Tr4 is the minimal binding motif of HR7A1.

FIGS. 10A, 10B illustrate that HR7A1 outcompetes Sgc4f for binding to α4β1-expressing cells.

FIGS. 11A, 11B illustrate that HR7A1.Tr2 selectively binds immortalized T-leukemia and T-lymphoma cancer cells over healthy PBMCs.

FIGS. 12A, 12B illustrate that HR7A1.Tr2 binds the majority of PBMCs.

FIGS. 13A, 13B illustrate that HR7A1.Tr2, fibronectin, and VCAM-1 share an overlapping binding epitope on α4β1.

FIG. 14 illustrates that mouse integrin α4 shares high sequence similarity with human α4 in beta-propeller domain.

FIG. 15 illustrates mouse integrin β1 shares high sequence similarity with human β1 in β1 and hybrid domains.

FIGS. 16A, 16B illustrate that HR7A1 binds mouse α4β1.

FIGS. 17A, 17B illustrate binding of HR7A1 and truncated variants in serum in this Example.

FIGS. 18A, 18B illustrate binding of HR7A1.Tr2 at physiological temperature.

FIG. 19 illustrates circulation half-lives of unmodified aptamers in vivo.

FIGS. 20A-20C illustrate that HR7A1.Tr2.S2E2 preserves binding affinity of HR7A1.Tr2.

FIGS. 21A-21C illustrate an example synthesis schematic for (21A) HPMA and AzP3MA monomers, (21B) biotin-PEG4-CCP chain transfer agent, and (21C) biotin-PEG4-(HPMA-co-AzP3MA)-CCP polymer. HPMA, 2-hydroxypropyl methacrylamide; AzP3MA; 11-azido-3,6,9-trioxaundecan-1-methacrylamide; PEG4, tetraethylene glycol; CCP, 4-Cyano-4-(dodecylsulfanylthiocarbonyl)sulfanylpentanoic acid.

FIG. 22 illustrates an example proton nuclear magnetic resonance (¹H-NMR) of AzP3MA monomer. AzP3MA; 11-azido-3,6,9-trioxaundecan-1-methacrylamide.

FIG. 23 illustrates an example ¹H-NMR of biotin-PEG4-CCP chain transfer agent. PEG4, tetraethylene glycol; CCP, 4-Cyano-4-(dodecylsulfanylthiocarbonyl)sulfanylpentanoic acid.

FIG. 24 illustrates an example ¹H-NMR characterization of biotin-PEG4-(HPMA-co-AzP3MA)-CCP polymerization at different timepoints. HPMA, 2-hydroxypropyl methacrylamide; AzP3MA; 11-azido-3,6,9-trioxaundecan-1-methacrylamide; PEG4, tetraethylene glycol; CCP, 4-Cyano-4-(dodecylsulfanylthiocarbonyl)sulfanylpentanoic acid.

FIG. 25 illustrates an example ¹H-NMR of biotin-PEG4-(HPMA-co-AzP3MA)-CCP polymer. HPMA, 2-hydroxypropyl methacrylamide; AzP3MA; 11-azido-3,6,9-trioxaundecan-1-methacrylamide; PEG4, tetraethylene glycol; CCP, 4-Cyano-4-(dodecylsulfanylthiocarbonyl)sulfanylpentanoic acid.

FIG. 26 illustrates an example gel permeation chromatogram of biotin-PEG4-(HPMA-co-AzP3MA)-CCP polymer. HPMA, 2-hydroxypropyl methacrylamide; AzP3MA; 11-azido-3,6,9-trioxaundecan-1-methacrylamide; PEG4, tetraethylene glycol; CCP, 4-Cyano-4-(dodecylsulfanylthiocarbonyl)sulfanylpentanoic acid.

FIGS. 27A-27G illustrate multivalent aptamer-polymer conjugates constructed with metal-free click chemistry address issues with aptamer serum and thermal stability.

FIG. 28 illustrates that higher valency aptamer-polymer conjugates display more non-specific cell binding.

FIG. 29 illustrates that aptamer-polymer conjugates have slightly altered circulation half-lives compared to free aptamers.

FIG. 30A illustrates that phosphorothioate-substituted HR7A1.Tr2.S2E2 aptamers PS3 and PSA retain binding to Jurkat cells, with apparent KD values of 3.2 and 9.6 nM, respectively. FIG. 30B illustrates that the phosphorothioate-substituted HR7A1.Tr2.S2E2.PSA aptamer displays improved thermal stability than the parental HR7A1.Tr2.S2E2 aptamer, retaining more binding to Jurkat cells at both 20° C. and 37° C. relative to its 4° C. binding.

FIGS. 31A and 31B illustrate that the more phosphorothioate-substituted HR7A1.Tr2.S2E2.PSA aptamer displays improved serum stability than the parental HR7A1.Tr2.S2E2 aptamer and the less phosphorothioate-substituted HR7A1.Tr2.S2E2.PS3 aptamer, displaying a serum half-life of 6.8 h compared <2 h for the other aptamers.

FIG. 32A illustrates that 100 nM and 1 μM HR7A1.Tr2 can inhibit Jurkat cell adhesion to VCAM-1 coated plates at 4° C. by >70% and >90%, respectively, regardless of the cells being pre-armed with aptamer or not. FIG. 32B illustrates that 100 nM HR7A1.Tr2.S2E2 and phosphorothioate-substituted HR7A1.Tr2.S2E2.PSA inhibit Jurkat cell adhesion to VCAM-1 coated plates less at 37° C. compared to 4° C. FIG. 32C illustrates that the 3:1 HR7A1.Tr2.S2E2-polymer conjugate displays improved inhibition of Jurkat cell adhesion to VCAM-1 coated plates at 37° C. compared to free aptamer, especially at lower concentrations.

### DETAILED DESCRIPTION

Various implementations of the present disclosure relate to oligonucleotides (e.g., aptamers) that specifically bind α4β1 (also known as VLA-4) and compositions thereof. For instance, an aptamer that specifically binds α4β1 is provided by SEQ ID NO: 1 (ATCCAGAGTGACGCAGCAAACCTGACCTCCTTACTAGATGCAACCCGACTACTAACGTCG TAAGAGAGCCTGGACACGGTGGCTTAGT). In some implementations, an oligonucleotide that specifically binds α4β1 is provided by SEQ ID NO: 5, SEQ ID NO: 6, SEQ ID NO: 8, or SEQ ID NO: 10. In some implementations, an oligonucleotide that specifically binds α4β1 includes the sequence as set forth in SEQ ID NO: 65 (ACTAGATGCAACCCGACTACTAACGTCGT). In some cases, the oligonucleotide is conjugated to a polymer, which can increase its stability, particularly in in vivo environments. The polymer, for example, includes N-(2-Hydroxypropyl) methacrylamide (HPMA) and/or 11-azido-3,6,9-trioxaundecan-1-methacrylamide (AzP3MA). In some examples, the polymer includes glycerol monomethacrylate.

Various compositions described herein can be utilized to increase the efficacy of a chemotherapeutic. Certain cancer cells that express α4β1 may be resistant to various cytotoxic chemotherapeutics. In some implementations, a therapeutically effective dosage of a composition including an oligonucleotide that specifically binds α4β1 can be administered to a subject with those cancer cells, thereby reducing their resistance to certain chemotherapeutic agents. According to various implementations, example chemotherapeutic agents enhanced by the administration of example compositions described herein include cytarabine; nilotinib; vincristine, dexamethasone, and L-asparaginase (VDL); doxorubicin; paclitaxel; and auristatin-based drugs.

Some compositions described herein can be utilized to treat one or more diseases. For example, α4β1 may be expressed by cells of a subject with multiple sclerosis (MS), sickle-cell disease, or T-leukemia. In various implementations of the present disclosure, administration of a therapeutically effective dosage of a composition including an oligonucleotide that specifically binds α4β1 can treat the disease of the subject. In some cases, some compositions can be used to inhibit α4β1 interactions and/or pathways to treat one or more diseases. For example, particular compositions may be used increase efficacy of immune therapies (also referred to as “immunotherapies”). In some examples, particular compositions can be used to block the α4β1 adhesion pathway to reduce migration of activated leukocytes to inflammation sites. In various cases, such as for treatment of multiple sclerosis (MS), some compositions can be used to decrease inflammation by blocking the α4-β1 integrin receptor that lines the blood-brain barrier, inhibiting leukocytes from entering the patient's central nervous system (CNS).

In addition, particular compositions can be utilized to sort and/or image cells that express α4β1. For instance, a composition may include an oligonucleotide that specifically binds α4β1 as well as a contrast agent, a radionucleotide, or some other detectable tag, such as a tag that can be imaged using one or more imaging modalities. In some cases, a composition includes an oligonucleotide that specifically binds α4β1 as well as a magnetic bead or other solid substrate. When exposed to the cells, the composition may be utilized to identify, image, or sort the cells.

Various implementations of the present disclosure offer several advantages over antibody-based compositions that specifically bind α4β1. For example, the oligonucleotides described herein can be more easily manufactured than antibodies that specifically bind α4β1. Oligonucleotides can be more easily conjugated to other compositions (e.g., polymers, substrates, therapeutically active compounds, etc.) than antibodies. Various oligonucleotides described herein are substantially smaller in size than many antibodies, which enables the oligonucleotides to more easily diffuse to targets. In various implementations, oligonucleotides exhibit “switchable” binding, such that the oligonucleotides can be selectively unbound to a target by introducing a reversal construct.

Further, various oligonucleotide-polymer compositions described herein provide advantages over other compositions. The polymer in an oligonucleotide-polymer composition can stabilize the oligonucleotide, particularly in the enzymatic, chemical, and thermal conditions of in vivo environments. Further, oligonucleotide-polymer compositions are more resistant to kidney-based filtration than bare oligonucleotides, due to their relative sizes. In some cases, oligonucleotide-polymer compositions can be constructed with multiple copies of the same oligonucleotide sequence, and may thereby have higher avidity for targets than single oligonucleotide molecules alone.

The present disclosure describes, for instance, an α4β1-specific DNA aptamer, named HR7A1, that selectively recognizes T-leukemia and lymphoma cell lines over healthy primary immune cells. The aptamer displays antibody-like affinity for both human and mouse α4β1-expressing malignant cells, outperforming other aptamers in its class, and retains its binding ability even after extensive truncation while blocking natural ligands like fibronectin and VCAM-1 from binding to α4β1. As expression of the α4β1 antigen is both associated with chemotherapy-refractory ALL and an adverse risk factor in childhood ALL at first relapse, and blocking binding of α4β1 to VCAM-1+ stromal cells can sensitize these cancer cells to chemotherapy, the HR7A1 aptamer holds great promise to synergize with drug payloads for targeted treatments of r/r leukemia and lymphoma, especially T-lineage malignancies that have high expression of this integrin.

Outside of cancer, α4β1 targeting has potential implications for the treatment and diagnosis of other disorders. Natalizumab is an integrin α4-specific antibody approved by the FDA for the treatment of multiple sclerosis and Crohn's disease (Polman et al., (2006) New England Journal of Medicine, 354, 899-910; Targan et al., (2007) astroenterology, 132, 1672-1683; Butzkueven et al., (2020) J Neurol Neurosurg Psychiatry, 91, 660-668). Natalizumab binds both α4β1 and α4β7, antagonizing their interaction with their respective adhesion molecules VCAM-1 and MAdCAM-1 to block lymphocyte migration to the central nervous system (CNS) and gastrointestinal tract, respectively (Kurmaeva, supra; Yu 2013, supra). While HR7A1 lacks binding to α4β7 and thus holds little promise for treating Crohn's disease, it may inhibit α4β1-dependent transmigration of circulating immune cells across the vascular endothelium into the CNS for the treatment of multiple sclerosis. Measuring the impact of HR7A1 on leukocyte adhesion to VCAM-1 coated plates and transmigratory capacity in an in vitro blood-brain barrier assay will be important to gauge the potential of the aptamer for this application (Khatri et al., (2009) Neurology, 72, 402-409). Besides autoimmune disorders, α4β1 has been shown to play an important role in sickle cell disease, mediating the adhesion of reticulocytes to inflamed endothelium that drive painful vaso-occlusive episodes (VOEs) (Swerlick et al., (1993) Blood, 82, 1891-189998). Accordingly, HR7A1 could be used in positron emission tomography (PET) imaging of VOEs or as an anti-adhesive therapy in sickle cell disease (Perkins et al., (2020) Blood Adv, 4, 4102-4112; White et al., (2016) Br J Haematol, 174, 970-982).

Various implementations of the present disclosure provide identification of aptamers that broadly binds to bulk PBMCs, which could have utility for cell sorting. For prospective ex vivo usage of HR7A1, traceless aptamer-based isolation strategies for cell therapy manufacturing have been previously developed (Kacherovsky 2019, supra; Cheng et al., (2022) ACS Appl Mater Interfaces, 14, 44136-44146). In these approaches, aptamers attached to solid supports (e.g., magnetic beads, chromatography resin) are used to positively enrich specific cells, and complementary reversal agents are used to recover label-free cells for downstream processing. While HR7A1 does not target a specific immune cell type, it broadly binds to bulk PBMCs, which could have utility for cell sorting. Commonly, the isolation of PBMCs from whole blood or other processed blood products involves density-gradient centrifugation, which is laborious and time-consuming. An approach that quickly isolates label-free PBMCs with few processing steps would thus greatly benefit research and clinical teams that use these cells for various applications. Accordingly, HR7A1 could prove useful in magnetic activated cell sorting (MACS) or affinity chromatography strategies with a complementary reversal agent to isolate label-free PBMCs from whole blood at low cost with high throughput.

In addition, the aptamers disclosed herein can be used for T-cell differentiation. For example, particular aptamers can be used to enhance T-cell lineage commitment of induced pluripotent stem cells (iPSCs) and CD34+ progenitors. In some cases, the aptamers described herein can be used as synthetic alternatives to VCAM1 in combination with Notch ligands for T-cell differentiation.

Aptamers are selected through a process known as Systematic Evolution of Ligands by EXponential enrichment (SELEX), in which a library of aptamer sequences is screened for binding to desired targets in iterative rounds of positive and negative selection. Targets used for SELEX can include small molecules, proteins, viruses, bacteria, cells, tissues, and even live animals (Ruscito and DeRosa. (2016) Front Chem, 4; Bayat et al., (2018) Biochimie, 154, 132-155; Gopinath et al., (2006) Journal of General Virology, 87, 479-487; Yu et al., (2018) J Biotechnol, 266, 39-49; Shangguan et al., (2006) Proceedings of the National Academy of Sciences, 103, 11838-11843; Li et al., (2021) Anal Chem, 93, 7369-7377; Cheng et al., (2013) Mol Ther Nucleic Acids, 2, e67). SELEX can be performed without knowledge of the target antigen. For example, cancer cell-specific aptamers can first be developed and then used to identify the target protein, leading to the discovery of novel biomarkers for targeted therapy (Shangguan et al., (2008) J Proteome Res, 7, 2133-9). Using SELEX, a portfolio of aptamers for targeting CD8+ T cells, monocytes, TfR1+ tumor cells, and SARS-CoV-2 was developed in just a short-span of five years (Kacherovsky et al., (2019) Nat Biomed Eng, 3, 783-795; Sylvestre et al., (2020) Bioconjug Chem, 31, 1899-1907; Cheng et al., (2022) J Am Chem Soc, 144, 13851-64; Kacherovsky et al., (2021) Angewandte Chemie Intl Ed, 60, 21211-21215).

Particular examples will now be described with reference to the accompanying figures. The scope of this disclosure includes individual examples described herein as well as any combination of the examples, unless otherwise specified.

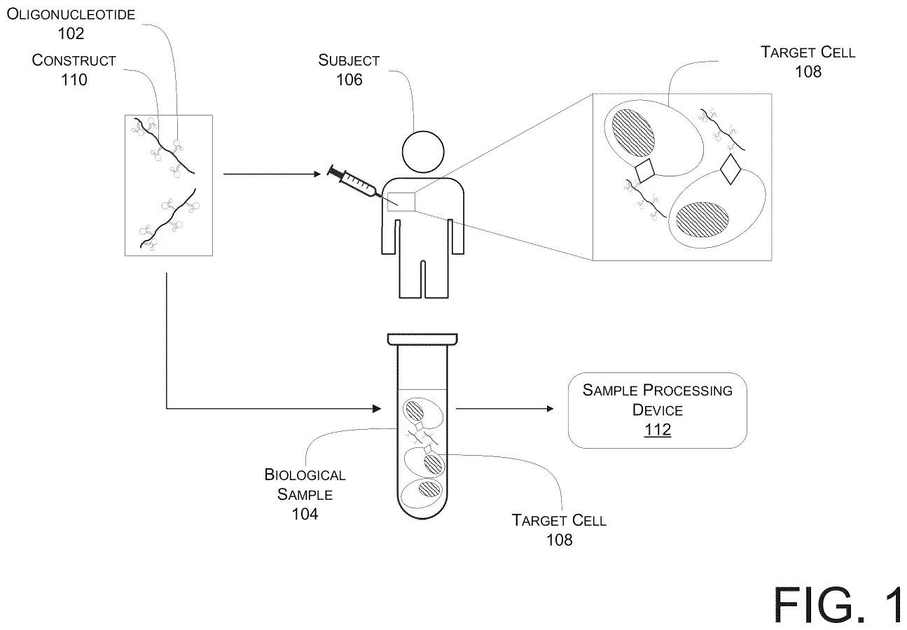

FIG. 1 illustrates an example environment 100 for using an oligonucleotide 102 that specifically binds α4β1. In various examples, a biological sample 104 may be taken from a subject 106. The subject 106 may be a human, a non-human primate, a mammal, or another animal. In various examples, a subject 106 suffers from a pathological disease associated with α4β1. For instance, the subject 106 has at least one of sickle-cell disease, an autoimmune disease (e.g., autoimmune encephalomyelitis, multiple sclerosis (MS), rheumatoid arthritis (RA), inflammatory bowel syndrome (IBS), T-cell mediated autoimmune disease), duchenne muscular dystrophy, dry eye disease, dry age-related macular degeneration, or a type of cancer. In some examples, the subject 104 has leukemia or lymphoma. In various cases, the subject 106 has a T-cell mediated disease, such as T-leukemia. The subject 106 may be undergoing treatment for the pathological disease associated with α4β1. For instance, the subject 106 may be receiving chemotherapy or an immune therapy.

α4β1, also referred to as very-late antigen-4 (VLA-4), is an integrin heterodimer that is involved in various cell functions, such as migration, growth, and survival, as well as inflammatory responses and tumor invasion. α4β1 is expressed on the cell surface of various cells, including leukocytes, stem cells, and progenitor cells. “Expression,” as used herein, can refer to the presence of a particular protein in a cell.

In various implementations, the biological sample 104 includes a mixture of cells. For instance, the biological sample 104 may include cells of more than one cell type. The biological sample 104 may include immune cells (e.g., lymphocytes, granulocytes, monocytes, etc.), red blood cells, epithelial cells, fat cells, muscle cells, stem cells, or a combination thereof. The biological sample 104 may include a target cell 108. In various implementations, the target cell 108 expresses α4β1. In some examples, α4β1 is expressed on the surface of the target cell 108. The target cell 108 is, in some examples, a leukocyte, a stem cell, or a progenitor cell. In various instances, the biological sample 104 includes a cell not expressing α4β1. The biological sample 104 may be a blood sample, a tissue sample, or another sample that includes cells of the subject 106.

In some cases, it may be possible to isolate an individual cell type from the biological sample 104 using antibodies that specifically bind to a marker expressed by the individual cell type. For example, anti-CD4 antibodies can be used to capture and isolate T cells in the biological sample 104 that express the CD4 glycoprotein. However, antibody-based cell selection techniques have limitations, including selection difficulties, selectivity problems (e.g., cross-reactivity, off-target binding, and batch-to-batch variation), preparation difficulties, high costs of production, stability issues, and lengthy production (e.g., manufacturing) times. Furthermore, due to the selectivity problems associated with antibodies, cell selection may involve more than one round of selection, including positive and/or negative selection. In certain cases, such as if the subject 106 has a condition that could cause serious and/or permanent harm to the subject 106 unless a treatment is administered quickly, minimizing processing time of the biological sample 104 may be particularly beneficial.

These issues can be addressed, for instance, by using the oligonucleotide 102 that specifically binds α4β1 to isolate cells that express α4β1. In various implementations, the oligonucleotide 102 is introduced to the biological sample 104 and/or the body of the subject 106 directly (e.g., intravenously), such as after the biological sample 104 has been subjected to one or more processing steps. The oligonucleotide 102, for instance, may spontaneously and specifically bind to α4β1 expressed by the target cell 108.

In some cases, it may be beneficial to inhibit α4β1 pathways and/or interactions associated with the pathological disease. In various instances, the efficacy of an immune therapy may be reduced due to immune cell adhesion that is mediated by α4β1. Inhibition of the α4β1 adhesion pathway may improve an immune response of the subject 106. In some cases, the target cell 108 may be a cancer cell that is resistant to a chemotherapy.

These issues can be addressed, in some cases, by administering, such as by injecting into the subject 106, the oligonucleotide 102 that specifically binds α4β1. The oligonucleotide 102 may be administered, in various instances, by intravenous injection, intravitreal injection, subcutaneous injection, peritoneal injection, intrathecal injection, or by intravenous infusion. Based on the administration of the oligonucleotide 102, α4β1 pathways and/or interactions, such as inflammatory responses or immune cell adhesion, may be reduced. In particular cases, the binding of the oligonucleotide 102 to the α4β1 expressed by various cells in the body of the subject 106 can at least partially inhibit an inflammatory response and/or immune cell adhesion. In various examples, the binding of the oligonucleotide 102 to cells in the body of the subject 106 can enhance the efficacy (e.g., reduce resistance) to one or more treatments subsequently administered to the subject 106. These treatments include, for instance, chemotherapy treatments and/or immunotherapy treatments.

The oligonucleotide 102, in various implementations, specifically binds α4β1. “Oligonucleotide” or “aptamer,” as used herein refers to a single-stranded nucleic acid. In some examples, the oligonucleotide 102 includes a sequence having at least 70% sequence identify to SEQ ID NO: 1, SEQ ID NO: 5, SEQ ID NO: 6, SEQ ID NO: 8, or SEQ ID NO: 10. In some implementations, a length of the oligonucleotide 102 is in a range of 22 to 150 nucleotides or in a range of 29 to 100 nucleotides. In some examples, the oligonucleotide 102 includes a sequence having at least 75%, 80%, 85%, 90%, 95%, 97%, or 99% sequence identify to SEQ ID NO: 1, SEQ ID NO: 5, SEQ ID NO: 6, SEQ ID NO: 8, or SEQ ID NO: 10. In some implementations, the oligonucleotide includes the sequence as set forth in SEQ ID NO: 1, SEQ ID NO: 5, SEQ ID NO: 6, SEQ ID NO: 8, or SEQ ID NO: 10.

In some cases, the oligonucleotide 102 is conjugated to a construct 110. The construct 110, in various examples, facilitates the detection or isolation of the oligonucleotide 102. In some implementations, the construct 110 facilitates the administration of the oligonucleotide 102 to the subject 106. For instance, the construct 110 may improve the in vitro or in vivo stability of the oligonucleotide 102. The construct 110 may include a polymer, a solid support, a tag, a linker, a protein, a lipid, a small molecule, or any combination thereof. In various cases, the polymer includes a water-soluble polymer (e.g., polyethylene glycol (PEG), multi-arm PEG, poly(N-isopropylacrylamide) (PNIPAM), polyethyleneimine (PEI), hydroxypropyl Methylcellulose (HPMC), or the like). In various examples, an oligonucleotide-to-polymer ratio is in a range of 1:1 to 10:1. Examples of a solid support include paper, glass, a polymer, a resin, a particle, or any combination thereof. Examples of a tag include a fluorophore, biotin, a dye, a chromophore tag, a quantum dot, a nanoparticle, a contrast agent, a radionuclide, another type of detectable label, or any combination thereof. In some examples, the oligonucleotide 102 is conjugated to a linker conjugated to a second oligonucleotide. For instance, the linker may link the oligonucleotide 102 to a second oligonucleotide 102 or a second oligonucleotide that binds a protein (e.g., albumin) to improve the stability or binding kinetics of the oligonucleotide 102. In some examples, the linker may link the oligonucleotide 102 to a small interfering RNA (siRNA) to manipulate genes associated with the pathological disease. For examples, the siRNA may cause the knock down of genes associated with the proliferation of cancer cells or genes associated with immune responses in autoimmune diseases. In various instances, the oligonucleotide 102 can be linked to a second oligonucleotide or to another nucleic acid (e.g., a siRNA, a microRNA (miRNA), an antisense oligonucleotide (ASO), or the like) without the linker. For example, the oligonucleotide 102 may include with a linker sequence (e.g., an overhanging palindrome sequence) that binds to the second oligonucleotide. The second oligonucleotide may include a sequence that is complementary to the linker sequence. In various implementations, the oligonucleotide 102 is conjugated to a lipid that is within a cell membrane, a liposome, a lipid nanoparticle, a microbubble, an extracellular vesicle, or any combination thereof. The lipid, in some cases, includes cholesterol. In some examples, the oligonucleotide 102 is conjugated to a small molecule that associates with a protein or polymer to extend the half-life of the oligonucleotide. For instance, the oligonucleotide 102 may be conjugated to Evans Blue. Evans Blue, in various instances, associates with albumin to improve the stability of the oligonucleotide 102.

The polymer may, in some instances, improve the stability of the oligonucleotide 102. For instance, based on conjugation to the polymer, an in vivo half-life of the oligonucleotide 102 may improve. The polymer, for example, includes HPMA and/or AzP3MA. For example, the polymer may be p(HPMA-co-AzP3MA) or p(GmMA-co-AzP3MA). In some examples, the polymer includes glycerol monomethacrylate. In various implementations, the oligonucleotide 102 includes one or more non-naturally occurring nucleotides or nucleotide analogs. In some examples, the backbone of the oligonucleotide 102 includes at least one phosphorothioate bond. For example, the oligonucleotide 102 may include 1, 2, 3, 4, 5, 6, 7, 8, 9, 10, 12, 15, 20, 25, 30, 35, or any other number of phosphorothioate bonds. The phosphorothioate bond may, in various instances, improve the stability of the oligonucleotide. In some instances, the backbone of the oligonucleotide 102 may include at least one phosphoramide linkage, a phosphorodithioate linkage, a boranophosphate linkage, a O-methylphosphoramidite linkage, a methylphosphorothioate linkage, a methylphosphonate linkage, or a peptide nucleic acid. In particular examples, the oligonucleotide 102 includes at least one locked nucleic acid (LNA). In some instances, the oligonucleotide 102 includes a 2′fluoro-arabino nucleic acid, a tricycle-DNA (tc-DNA), a peptide nucleic acid, a cyclohexene nucleic acid (CeNA), a locked nucleic acid (LNA) nucleotide comprising a methylene bridge between the 2′ and 4′ carbons of the ribose ring, a bridged nucleic acid (BNA), a ethylene-bridged nucleic acid (ENA), a phosphodiamidate morpholino, or a combination thereof. In particular examples, the non-naturally occurring nucleotides or nucleotide analogs include 2′-O-methyl analogs, 2′-deoxy analogs, 2-thiouridine analogs, N6-methyladenosine analogs, or 2′-fluoro analogs. For example, the oligonucleotide 102 may include at least one nucleotide with a fluorine molecule at the 2′ ribose position instead of a 2′-hydroxyl group. In various implementations, a number of linkages, non-naturally occurring nucleotides, or nucleotide analogs is in a range of 1 to 150. In some examples, the number of linkages, non-naturally occurring nucleotides, or nucleotide analogs is in a range of 5 to 50.

In some cases, the oligonucleotide 102 may include one or more nucleobase-modified ribonucleotides. The one or more modified ribonucleotides may include a non-naturally occurring base (instead of a naturally occurring base), such as uridines or cytidines modified at the 5′-position (e.g., 5′ (2-amino)propyl uridine or 5′-bromo uridine); adenosines and guanosines modified at the 8-position (e.g., 8-bromo guanosine); deaza nucleotides (e.g., 7-deaza-adenosine); and N-alkylated nucleotides (e.g., N6-methyl adenosine). The nucleobase-modified nucleotides may include an aminopurine, 2,6-diaminopurne (2-Amino-dA), 5-Bromo deoxyuridine (dU), deoxyuridine, Inverted deoxythymidine (dT), Inverted Dideoxy-T, dideoxy-C, 5-Methyl dC, Super (T), Super (G), 5-Nitroindole, 2′-O-Methyl base, 2′O-methoxyl ethyl base, 2′ methyl base, Hydroxymetyl deoxycytosine (dC), Iso deoxyguanosine (dG), Iso dC, Fluoro C, Fluoro U, Fluoro A, Fluoro G, 2-MethoxyEthoxy MeC, 2-MethoxyEthoxy G, or 2-MethoxyEthoxyT.

In some cases, the 3′ and 5′ termini of the oligonucleotides can be substantially protected from nucleases, e.g., by modifying the 3′ or 5′ linkages. For example, the oligonucleotides can be made resistant by the inclusion of one or more blocking groups. The one or more end-blocking groups can be a cap structure (e.g., a 7-methylguanosine cap), inverted nucleomonomer (e.g., with 3′-3′ or 5′-5′ end inversions), methylphosphonate, phosphoramidite, non-nucleotide groups (e.g., non-nucleotide linkers, amino linkers, conjugates) and the like. The 3′ terminal nucleomonomer can comprise a modified sugar moiety. For example, the 3′-hydroxyl can be esterified to a nucleotide through a 3′→3′ internucleotide linkage. For example, the alkyloxy radical can be methoxy, ethoxy, or isopropoxy. Optionally, the 3′→3′ linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. In particular examples, the oligonucleotide 102 includes a 3′ Inverted deoxythymidine (dT) modification to increase nuclease resistance of the oligonucleotide 102.

In some implementations, the oligonucleotide 102 is conjugated to a detectable label for detection of the target cell 108 in the subject 106 or in the biological sample 104. For example, the construct 110 may be a contrast agent, radionuclide, a dye, or any combination thereof. In some examples, the contrast agent is an iodine-based contrast agent, a gadolinium-based contrast agent, a microbubble, barium sulfate, or another contrast agent. In some examples, the contrast agent is an iodine-based contrast agent, a gadolinium-based contrast agent, a microbubble, barium sulfate, or another contrast agent. In some examples, the radionuclide may be fluorine-18 (F-18), yttrium-90 (Y-90), carbon-11 (C-11), gallium-68 (Ga-68), technetium-99m (Tc-99m), iodine-123 (I-123), Iodine-131 (I-131), Indium-111 (In-111), xenon-133 (xe-133), thallium-201 (Tl-201), or another radionuclide. In some examples, the dye may be a gadolinium-based dye, an iodine-based dye, barium sulfate, fluorescein, indocyanine green, or another dye. In various implementations, the conjugated oligonucleotide 102 is administered to the subject. The oligonucleotide 102 may bind the target cell 108 in the subject. The target cell 108 may be detected by performing medical imaging, such computed tomography (CT) imaging, positron emission tomography (PET) imaging, magnetic resonance imaging (MRI), single photon emission computed tomography (SPECT), other medical imaging techniques, or any combination thereof, on the subject 106.

In some examples, the detectable label includes a fluorophore, biotin, a dye, a chromophore tag, a liposome, a micelle, or a nanoparticle (a quantum dot, a metal nanoparticle, a metal oxide nanoparticle, a polymeric nanoparticle, a carbon-based nanoparticle, another type of nanoparticle, or any combination thereof), and the conjugated oligonucleotide 102 may be applied to the biological sample 104. The target cell 108 may be detected using one or more sample processing device(s) 112 configured to detect the detectable label. The target cell 108 can be detected by visual inspection or using spectroscopy, surface-enhanced Raman scattering, surface plasmon resonance, or another appropriate detection technique. In some instances, the oligonucleotide 102 is conjugated to biotin, and the target cell 108 is detected using a streptavidin-conjugated label. In some examples, the oligonucleotide 102 is conjugated to a fluorophore, and the target cell 108 is detected using the sample processing device(s) 112 (e.g., a microscope, a fluorometer, a plate reader, or another imaging system). In some examples, the sample processing device(s) 112 is a medical imaging device, such as MRI scanner, a CT scanner, an ultrasound machine (e.g., a sonography machine), or the like.

In various implementations, the biological sample 104 is exposed to the oligonucleotide 102 to isolate the target cell 108. In various implementations, cell selection using oligonucleotides offers several benefits over antibody-based techniques, including simple and inexpensive production, strong specificity and discrimination of molecular differences, longer shelf life, batch-to-batch consistency, and capacity for chemical modification. For instance, the subject 106 may benefit from the removal of the target cell 108. Based on exposing the biological sample 104 to the oligonucleotide 102, the oligonucleotide 102 may bind the target cell 108. Based on removing the target cell 108 from the biological sample 104, the biological sample 104 may be administered to the subject 106. In various implementations, a binding affinity of the oligonucleotide 102 to the target cell 108 is in a range of 10 nanomolar (nM) to 1 micromolar (μM). The binding affinity, in some cases, is in a range of 100 nM to 1 μM. The target cell 108 is, in some cases, a lymphocyte.

In some examples, the biological sample 104 is incubated with the oligonucleotide 102. The oligonucleotide 102 may be, in various cases, conjugated to the construct 110. For example, a solution including the mixture of cells may be produced from the biological sample 104. In some instances, the solution may be produced by generating a cell suspension from the biological sample 104, filtering the biological sample 104, diluting the biological sample 104, washing the biological sample 104 with a buffer and/or performing lysis of one or more cells that do not express α4β1 (e.g., red blood cells, cells not expressing α4β1, etc.). In various cases, filtering can be done manually or using a machine. Filtering may include gravity filtration, centrifugation, vacuum filtration, pressure filtration, microfiltration, or another suitable filtration method. In some examples, the oligonucleotide 102 is conjugated to a magnetic bead (e.g., the construct 110) and incubated with the solution for a time. The time, in some cases, is determined based on binding kinetics of the oligonucleotide 102 and α4β1. During the time, the oligonucleotide 102 may spontaneously bind the target cell 108.

In some examples, it may be beneficial to isolate cells expressing more than one antigen. For instance, it may be beneficial to isolate cells expressing a first antigen (e.g., α4β1) and a second antigen (e.g., CD4, CD8, etc.). For example, it may be beneficial to isolate cells expressing α4β1 and CD8 (e.g., CD8+ α4β1+ T cells).

In various implementations, a second solution including the target cell 108 is exposed to a third oligonucleotide that specifically binds the second antigen. In some examples, an antibody that specifically binds the second antigen is used instead of the third oligonucleotide. The second antigen, in various examples, is associated with the pathological disease. For example, the second antigen may be CD8. In some examples, the second antigen may be CD4. In some examples, the third oligonucleotide is conjugated to a disclosed support, a disclosed tag, or a disclosed linker. In some cases, a cell expressing α4β1 and the second antigen binds to the third oligonucleotide. The bound cell expressing α4β1 and the second antigen can be isolated as described herein. In particular examples, the third oligonucleotide can be used to isolate individual immune cell populations (e.g., CD4+ T cells, CD8+ T cells, monocytes, etc.).

The target cell 108 is isolated, in some examples, by physically manipulating and/or isolating the construct 110 from the solution. For instance, the oligonucleotide 102 may be conjugated to a magnetic bead, and the target cell 108 may be isolated applying a magnetic field to the solution. For instance, the magnetic field may be produced by a ferromagnetic material and/or an electromagnet. For example, the solution may be in a container, and a ferromagnetic material may be placed on the outer surface of the container. The magnetic field causes the magnetic beads to align with the magnetic field and form a cluster within the solution. In some examples, the magnetic beads aggregate toward a pole of a magnetic material or the source of the magnetic field. In some examples, the aggregated magnetic beads can be removed to isolate the cells expressing α4β1 108. In some examples, the aggregated magnetic beads can be immobilized by the magnet and the cells not expressing α4β1 may be removed by decanting, draining, aspirating, washing, or another suitable method. The sample processing device(s) 112 may include a device configured to apply a magnetic field to the solution.

In some examples, the oligonucleotide 102 is conjugated to a microbubble (e.g., the construct 110) including a shell encapsulating a gas. In some cases, the shell can include lipids, proteins, polymers (e.g., poly(lactic co-glycolic acid) (PLGA)), lipopolymers, or phospholipids. The gas, in various examples, may include air, nitrogen, or a perfluorocarbon (e.g., perfluorobutane or perfluoropropane). A size of the microbubble, in some examples, is in a range of 1 to 10 μm. The oligonucleotide 102 may be mixed into the solution derived from the biological sample 104. Based on mixing the oligonucleotide 102 into the solution, the oligonucleotide 102 may bind the target cell 108. Due to the buoyancy of the microbubble, the target cell 108 may float to the surface of the solution. In some cases, the target cell 108 is isolated by aspiration. “Aspiration,” as used herein, can refer to the process of collecting a component of a solution, wherein the component is generally located at the surface of the solution. In various examples, the aspiration can be performed manually (e.g., using a pipette or syringe), by using a vacuum aspiration system, or by using another appropriate system.

In various examples, the oligonucleotide is immobilized to paper, glass, or a polymer (e.g., a solid support, the construct 110). The solution derived from the biological sample 104 may be washed over the solid support or incubated with the solid support. In various instances, the target cell 108 binds to the oligonucleotide 102. The target cell 108 is isolated, for instance, by removing the solid support from the solution or by applying a buffer to the solid support to remove one or more cells not expressing α4β1.

In some examples, the oligonucleotide 102 is conjugated to one or more fluorophores. In various cases, the oligonucleotide 102 is conjugated to two or more fluorophores. The solution derived from the biological sample 104 may be incubated with the oligonucleotide 102, and the oligonucleotide 102 may bind the target cell 108. The binding of the oligonucleotide 102 to the target cell 108, for instance, causes a conformational change of the oligonucleotide 102. Based on the conformational change, a distance between the two fluorophores may decrease, causing a transfer of energy between the two fluorophores. Based on the transfer of energy, the fluorescence emission spectra of the oligonucleotide 102 may change. The target cell 108 can be isolated by detecting the fluorescence emission spectra. In some examples, the target cell 108 is isolated by performing fluorescence activated cell sorting (FACS). In various instances, the sample processing device(s) 112 include one or more devices described herein. For example, the sample processing device(s) 112 may include a fluorescence activated cell sorter or a flow cytometer.

In various implementations, based on the target cell 108 being bound to the oligonucleotide 102, the target cell 108 is dissociated from the oligonucleotide 102. The target cell 108 can be dissociated from the oligonucleotide 102, for example, by introducing a reversal construct to the solution, changing a pH of the solution (e.g., by adding an acid or a base to the solution), changing a concentration of salt in the solution, changing a concentration of chelators in the solution, changing a temperature of the solution, applying a mechanical force to the solution, or introducing a nuclease enzyme to the solution, or changing the composition of the solution in any suitable manner. In various implementations, any change in the pH of the solution is sufficient to dissociate the target cell 108 from the oligonucleotide 102. In some examples, changing the solution to be more basic may dissociate the target cell 108 from the oligonucleotide 102. In some examples, changing the solution to be more acidic may dissociate the target cell 108 from the oligonucleotide 102. In various examples, the pH of the solution may be increased by 0.1, 0.2, 0.5, 1, 2, 3, 4, 5, 6, 7, 8, 9, or 10 to dissociate the target cell 108 from the oligonucleotide 102. In various examples, the pH of the solution may be decreased by 0.1, 0.2, 0.5, 1, 2, 3, 4, 5, 6, 7, 8, 9, or 10 to dissociate the target cell 108 from the oligonucleotide 102. In various implementations, the mechanical force can be applied manually or using a machine (e.g., an orbital shaker, a platform shaker, a shaking incubator, or another suitable machine). In some examples, the target cell 108 is dissociated from the oligonucleotide 102 before the target cell 108 is introduced to the third oligonucleotide that specifically binds the second antigen.

In various implementations, the target cell 108 is removed from the solution for treatment of the pathological disease. For instance, a clinician may determine that the subject 106 suffers from a pathological disease associated with α4β1, and that the subject 106 may benefit from the removal of the target cell 108. In some cases, based on isolating the target cell 108 using the oligonucleotide 102, the solution is reintroduced into the subject 106. For instance, the target cell 108 may be removed from the solution, and the solution may be administered intravenously (e.g., via intravenous infusion) to the subject 106.

In some examples, the subject 106 may benefit from a treatment that includes the target cell 108. For instance, the target cell 108 may be a T cell that can be modified to treat the pathological disease of the subject 106. In various instances, the subject 106 may benefit from a treatment that includes a cell that is differentiated using the oligonucleotide 102. For instance, the oligonucleotide 102 may be applied to induced pluripotent stem cells (iPSCs) and/or CD34+ progenitors during cell therapy manufacturing to induce T-cell differentiation. For instance, a stem cell may be differentiated into a T cell using the oligonucleotide 102 that is conjugated to a microbead. In various cases, the stem cell is exposed to the oligonucleotide 102 and Delta-like ligand 4 (DLL-4) to induce differentiation into T cells or natural killer (NK) cells. In some examples, the target cell 108 is a T cell, and based on isolating the T cell, the T cell may be modified with a CAR for treatment of the pathological disease.

In various implementations, the target cell 108 or a T cell differentiated using the oligonucleotide 102 is modified to treat the subject 106. For example, the subject 106 may have a type of cancer, and cancer cells expressing an antigen may be present in the subject 106. The target cell 108 or the T cell differentiated using the oligonucleotide 102 is, in some examples, transduced with a vector encoding a chimeric antigen receptor (CAR). In some cases, the CAR is designed to specifically bind the antigen expressed by the cancer cells of the subject 106. In various examples, the target cell 108 or the T cell differentiated using the oligonucleotide 102 transduced with the CAR is a CAR T cell. The CAR T cells may be infused into the subject 106, and the CAR may bind to one or more cancer cells in the subject. Based on binding the one or more cancer cells, the CAR may activate the CAR T cell to cause cell death of the one or more cancer cells.

In particular implementations, treatments described herein (e.g., CAR T cells, the oligonucleotide 102, the oligonucleotide 102 conjugated to the construct 110) can be formulated into a carrier in a therapeutically-effective amount. As described herein, exemplary carriers include saline, buffered saline, physiological saline, water, Hanks' solution, Ringer's solution, Normosol-R (Abbott Labs), PLASMA-LYTE A® (Baxter Laboratories, Inc., Morton Grove, IL), and combinations thereof.

Cells (e.g., the target cell 108, CAR T cells), compositions including the oligonucleotide 102, and or other components described herein may be administered in a formulation that includes one or more carriers, stabilizers, anesthetics, preservatives, or any combinations thereof.

In particular implementations, carriers can be supplemented with human serum albumin (HAS) or other human serum components or fetal bovine serum. In particular implementations, a carrier for infusion includes buffered saline with 5% HAS or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol;

PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HAS, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where beneficial, formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

In some examples, the subject 106 may benefit from administration of the oligonucleotide 102. For instance, a clinician may determine that the subject 106 has a condition (e.g., a type of cancer) may benefit from one or more treatments, such as an immunotherapy and/or a chemotherapy. The chemotherapy, in various instances, includes a cytotoxic chemotherapy such as cytarabine; nilotinib; vincristine, dexamethasone, L-asparaginase (VDL); doxorubicin; paclitaxel; an auristatin; or another cytotoxic chemotherapy. In various implementations, the oligonucleotide 102 may be administered to the subject 106 concurrently with, or before administration of, the treatment(s). In some cases, the oligonucleotide 102 is administered before or after an administration of the treatment(s). For instance, the oligonucleotide 102 may be administered before a dose of the treatment(s) is administered to the subject 106. In various instances, the oligonucleotide 102 is administered after the subject 106 has completed a course of the treatment(s). In some examples, the oligonucleotide 102 is linked to a drug associated with the treatment or to a carrier (e.g., a polymer) that is linked to the drug associated with the treatment. Based on the administration of the oligonucleotide 102, the efficacy of the treatment(s) improve. For example, the efficacy of the treatment(s) may be determined based on a condition of the subject 106, a prognosis of the subject 106, one or more biomarkers associated with the pathological disease in the subject 106, one or more symptoms of the subject 106, or another metric associated with the pathological disease. In some examples, the subject 106 may benefit from administration of the oligonucleotide 102 due to the inhibition of the target cell 108 in the subject 106. For instance, based on the administration of the oligonucleotide, an inflammatory response or an hasmmune suppression associated with α4β1 may be reduced in the subject 106. Accordingly, the condition of the subject 106 may improve.

In some examples, the oligonucleotide 102 is conjugated to a drug for the treatment of the pathological disease. The drug may be a pharmaceutical drug or a therapeutic agent that is administered to the subject 106 for the purpose of diminishing or eliminating signs or symptoms of the pathological disease. For example, the subject 106 may have an autoimmune disease and may benefit from the destruction of cells expressing α4β1 in the subject 106. In some examples, the drug is a cytotoxic drug. Examples of cytotoxic drugs include actinomycin D, an alkylating agent, anthracycline, auristatin, calicheamicin, camptothecin, CC1065, 21olchicine, cyclophosphamide, cytarabine, cytochalasin B, daunorubicin, 1-dehydrotestosterone, dihydroxy anthracinedione, dolastatin, doxorubicin, duocarmycin, elinafide, emetine, ethidium bromide, etoposide, gramicidin D, glucocorticoids, lidocaine, maytansinoid, mithramycin, mitomycin, mitoxantrone, nemorubicin, PNU-159682, procaine, propranolol, puromycin, pyrrolobenzodiazepine, taxane, taxol, tenoposide, tetracaine, trichothecene, vinblastine, vinca alkaloid, or vincristine. A therapeutically effective amount of the oligonucleotide 102 may be administered to the subject 106. Based on the administration of the oligonucleotide 102, the drug may provide a cytotoxic effect on the cells expressing α4β1.

Treating subjects includes delivering therapeutically effective amounts. Therapeutically effective amounts include those that provide effective amounts, prophylactic treatments and/or therapeutic treatments.

An “effective amount” is the amount of a formulation used to result in a desired physiological change in the subject. For example, an effective amount can provide a change in a metric associated with a pathological disease associated with α4β1 (e.g., a severity or frequency of symptoms or a change in a marker associated with the pathological disease). Effective amounts are often administered for research purposes. Effective amounts disclosed herein can cause a statistically significant effect in an animal model or in vitro assay relevant to the assessment of a pathological disease's development or progression.

A “prophylactic treatment” includes a treatment administered to a subject (e.g., the subject 106) who does not display signs or symptoms of a pathological disease associated with α4β1 or displays only early signs or symptoms of a pathological disease associated with α4β1 such that treatment is administered for the purpose of diminishing or decreasing the risk of developing the pathological disease further. Thus, a prophylactic treatment functions as a preventative treatment against a pathological disease associated with α4β1. In particular embodiments, prophylactic treatments reduce or delay physical symptoms associated with a pathological disease associated with α4β1.

A “therapeutic treatment” includes a treatment administered to a subject (e.g., the subject 106) who displays symptoms or signs of a pathological disease associated with α4β1 and is administered to the subject for the purpose of diminishing or eliminating those signs or symptoms of the pathological disease associated with α4β1. The therapeutic treatment can reduce, control, or eliminate the presence or activity of the pathological disease associated with α4β1 and/or reduce control or eliminate side effects of the pathological disease associated with α4β1.

Function as an effective amount, prophylactic treatment or therapeutic treatment are not mutually exclusive, and in particular embodiments, administered dosages may accomplish more than one treatment type.

In particular embodiments, therapeutically effective amounts induce a reduced progression of a pathological disease associated with α4β1. In particular embodiments, the reduced progression includes reduced or stabilized symptoms, reduced or stabilized levels of one or more markers associated with the pathological disease, or a change in another metric known in the art.

For administration, therapeutically effective amounts (also referred to herein as “doses” or “dosages”) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of condition, stage of condition, previous or concurrent therapeutic interventions, idiopathy of the subject and route of administration.

Useful doses can range from 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Exemplary doses of cell-based compositions can include 104 to 109 cells/kg body weight, or 10³ to 10¹¹ cells/kg body weight. Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular embodiments, the treatment protocol may be dictated by a clinical trial protocol or an FDA-approved treatment protocol.

Various compositions described herein can be administered by, for example, injection, inhalation, infusion, perfusion, lavage, or ingestion. Routes of administration can include intravenous, intravitreal, peritoneal, intradermal, intraarterial, intranodal, intravesicular, intrathecal, intraperitoneal, intraparenteral, intranasal, intralesional, intramuscular, oral, subcutaneous, and/or sublingual administration. Formulations are generally be administered by injection. In some examples, formulations may be administered by intravenous infusion.

In various implementations of the present disclosure, a composition including the oligonucleotide 102 can be administered by, e.g., injection, infusion, perfusion, or lavage. Routes of administration can include bolus intravenous, intradermal, intraarterial, intraparenteral, intranodal, intralymphatic, intraperitoneal, intralesional, intraprostatic, intravaginal, intrarectal, topical, intrathecal, intratumoral, intramuscular, intravesical, and/or subcutaneous administration.

In some implementations, the oligonucleotide 102 is administered with a

pharmaceutically acceptable carrier. Exemplary pharmaceutically acceptable carriers and formulations are disclosed in Remington's Pharmaceutical Sciences, 18^{th} Ed. Mack Printing Company, 1990. Moreover, formulations can be prepared to meet sterility, pyrogenicity, general safety and purity standards as required by United States FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants.

Exemplary buffering agents include citrate buffers, succinate buffers, tartrate buffers, fumarate buffers, gluconate buffers, oxalate buffers, lactate buffers, acetate buffers, phosphate buffers, histidine buffers and/or trimethylamine salts.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol and 3-pentanol.

Exemplary isotonic agents include polyhydric sugar alcohols including trihydric or higher sugar alcohols, such as glycerin, erythritol, arabitol, xylitol, sorbitol or mannitol.

Exemplary stabilizers include organic sugars, polyhydric sugar alcohols, polyethylene glycol, sulfur-containing reducing agents, amino acids, low molecular weight polypeptides, proteins, immunoglobulins, hydrophilic polymers or polysaccharides.

In some implementations, the oligonucleotide 102, the construct 102, and other elements described herein can be included in one or more kits. Kits can include various components to practice methods disclosed herein. For example, kits can include the oligonucleotide 102, the construct 110, α4β1, a nucleic acid encoding α4β1, a chimeric antigen receptor (CAR), a nucleic acid encoding a CAR, cells (e.g., immune cells, T-cells, B cells, natural killer (NK) cells, NK-T-cells, monocytes/macrophages, lymphocytes, hematopoietic stem cells (HSCs), hematopoietic progenitor cells (HPC), and/or a mixture of HSC and HPC (i.e., HSPC), untransduced T-cells, CAR T-cells); cell lines (e.g., J.RT3-T.35, Jurkat, and H9 cell lines); tissue samples (e.g., specimens, or other organ, and/or cells derived therefrom); genetic expression components (e.g., genes for expression provided by vectors (e.g., lentiviral vector, retroviral vector), CRISPR components, ZFNs, TALENs, MegaTALs, targeted viral vectors and/or nanoparticles); cell formulations or activation components (e.g., saline, buffered saline, phosphate buffered saline (PBS); biocompatible buffers such as, Ca++/Mg++ free PBS; physiological saline, water, Hanks' solution, Ringer's solution, T-cell stimulating epitopes (e.g., anti-CD3/anti-CD28 conjugated beads; OKT3, TGN1412), culture-initiating compositions, RPMI medium, non-essential amino acids, sodium pyruvate, penicillin/streptomycin, non-dividing EBV-transformed lymphoblastoid cells (LCL), IL-21, human serum albumin (HAS) or other human serum components or fetal bovine serum, dextrose, Stabilizers, preservatives); components for screening form KMT2A fusion (e.g., fusion probe, KMT2A probe, fluorescent-labeled nucleotide analog, microscope, FISH analytics software); combination therapy components (e.g., local anesthetics, chemotherapeutic agents, immunosuppressive agents, anti-inflammatory agents); an antibody tagged with a fluorescent molecule; PCR amplification sequences; cytokines (e.g., IL-2, IL-7, IL-15, IL-21); culture vessels; GAPDH; IFN-γ enzyme-linked immunosorbent assay (ELISA); culture plates; etc.

### Experimental Example 1

### Materials and Methods

Oligonucleotides, buffers, and aptamer folding. All oligonucleotides used were synthesized by Integrated DNA Technologies (Coralville, IA). Both the ssDNA library used in cell-SELEX and the primers used for library amplification between SELEX rounds are as previously described (Kacherovsky et al., (2019) Nat Biomed Eng, 3, 783-795). The individual synthesized ssDNA aptamers are listed in Table 1. Wash buffer was prepared from DPBS with calcium and magnesium (Corning of Corning, NY) supplemented with 4.5 g/L glucose and 5 mM MgCl², as previously described (Sefah et al., (2010) Nat Protoc, 5, 1169-85). Binding buffer for flow cytometry studies was prepared from wash buffer that was further supplemented with 1% bovine serum albumin (BSA) weight per volume (w/v) and 0.1 mg/mL yeast tRNA (Invitrogen of Carlsbad, CA). Before binding, aptamers were folded by preparing a 1 μM stock in wash buffer and heating at 95° C. for 5 min followed by snap-cooling on ice.

TABLE 1
Aptamer sequences

| Name | Sequence |
|---|---|
| RN | 5′-*ATCCAGAGTGACGCAGCA*AATTCCAAACTCGAGTAAGCGTAGAGCCTCTCAT |
| | CGCCTCAATAA*TGGACACGGTGGCTTAGT-3′ (SEQ ID NO: 2) |
| HR7A1 | 5′-*ATCCAGAGTGACGCAGCA*AACCTGACCTCCTTACTAGATGCAACCCGACTAC |
| | TAACGTCGTAAGAGAGCC*TGGACACGGTGGCTTAGT-3′ (SEQ ID NO: 1) |
| tJBA8.1 | 5′-*GCAGCA*GCGTAAAGGGGGTGTTTGTGCGGTGTGGAGTGCGCGTGCTGCTG |
| | C-3′ (SEQ ID NO: 3) |
| CD8.A3 | 5′-*ATCCAGAGTGACGCAGCA*ACAGAGGTGTAGAAGTACACGTGAACAAGCTTGA |
| | AATTGTCTCTGACAGAGG*TGGACACGGTGGCTTAGT-3′ (SEQ ID NO: 4) |
| HR7A1.Tr1 | 5′-*CAGCA*AACCTGACCTCCTTACTAGATGCAACCCGACTACTAACGTCGTAAGA |
| | GAG-3′ (SEQ ID NO: 5) |
| HR7A1.Tr2 | 5′-*CTCCTTACTAGATGCAACCCGACTACTAACGTCGTAAGAGAG-3′ |
| | (SEQ ID NO: 6) |
| HR7A1.Tr3 | 5′-CTAGATGCAACCCGACTACTAACGTCG-3′ (SEQ ID NO: 7) |
| HR7A1.Tr4 | 5′-GCGTACTAGATGCAACCCGACTACTAACGTCGTACGCG-3′ |
| | (SEQ ID NO: 8) |
| Sgc4f | 5′-ATCACTTATAACGAGTGCGGATGCAAACGCCAGACAGGGGGACAGGAGATAAGT |
| | GA-3′ (SEQ ID NO: 9) |
| HR7A1.Tr2.S2E2 | 5′-CGCGCCGTACTAGATGCAACCCGACTACTAACGTCGTACGAGCGCG-3′ |
| | (SEQ ID NO: 10) |

Table 1 describes the sequences of aptamers used in this Example. Aptamers were modified on their 5′ and/or 3′-end with 6-FAM, Cy5, biotin-hexa-ethyleneglycol, and/or DBCO depending on the assay. The exact modifications are specified in the figures and captions. Constant regions are surrounded by asterisks. Nucleotides that were gradually deleted to make the truncated aptamer variants are: positions 1-33 and 61-88 of HR7A1; positions 1-20 and 48-55 of HR7A1.Tr1; positions 1-7 and 35-42 of HR7A1.Tr2; positions 2, 4, 5, 33, 34, and 36 of HR7A1.Tr4; and positions 3, 5, 6, 8, 9, 37, 38, 40-42, and 44 of HR7A1.Tr2.S2E2. nucleotides that were added to are: positions 1 and 37 of HR7A1.Tr4; and positions 1, 2, 45, and 46 of HR7A1.Tr2.S2E2. Nucleotides that were substituted into aptamer sequences are: positions 3 and 35 of HR7A1.Tr4; and positions 4, 7, 39, and 43 of HR7A1.Tr2.S2E2.

Antibodies and flow cytometry. The following dyes and antibodies were used for cell staining: Zombie Violet (1:500 in 100 μL per 10⁶ cells, BioLegend of San Diego, CA), APC anti-human CD276 (1:200, 351006, BioLegend), FITC anti-human CD49d (1:50, 304315, BioLegend), PE anti-human CD29 (1:50, 303004, BioLegend), PE anti-mouse CD49d (1:50, 103607, BioLegend), PE anti-mouse/rat CD29 (1:50, 102207, BioLegend), Alexa Fluor 647 streptavidin (1:500, BioLegend), and APC anti-His tag (1:20, 362605, BioLegend). Stained cells were assessed on an Attune NxT Flow Cytometer (Life Technologies of Carlsbad, CA), and data was analyzed and plotted in FlowJo V10 software (Becton Dickinson of Franklin Lakes, NJ). The median fluorescence intensity of singlet live cell events was used as a measurement of aptamer binding. Binding curves, KD values, and graphs were generated using GraphPad Prism 9 software (San Diego, CA).

Cloning of 4IgB7H3 constructs and lentivirus production. The DNA sequence for 4IgB7H3 (UniProtKB: Q5ZPR3-1) was synthesized and cloned into epHIV7.2 lentiviral vectors by GeneArt (Regensburg, Bayern, Germany) using NheI and NotI restriction enzymes. 5-alpha chemically competent E. coli (New England Biolabs, NEB of Ipswich, MA) were transformed with the resulting plasmid and selected by kanamycin. Correct cloning was verified by sangar sequencing (GENEWIZ of Plainfield, NJ) of miniprep DNA (QIAGEN of Hilden, Germany) before transfection-grade plasmid DNA was prepared by maxiprep (MACHEREY-NAGEL of Düren, Nordrhein-westfalen, Germany).

HEK293T cells were purchased from ATCC and used before passage 20. HEK 293T cells were seeded 24 h prior to transfection in eight 10 cm plates at 3×10⁶ cells per plate in 8 mL virus prep medium comprised of DMEM with high-glucose, L-glutamine, and sodium pyruvate (Gibco of Grand Island, NY) supplemented with 10% heat-inactivated FBS (VWR of Radnor, Pennsylvania). The next day, each plate was transfected with 40 μL Lipofectamine 2000 reagent (Life Technologies) mixed with 1000 μL Opti-MEM containing transgene lentiviral vector (15 μg), pCMV-Rev2 (1 μg), pCHGP-2 (10 μg), and pCMV-G (2 μg). After 24 h, the top media was aspirated and replaced with virus prep medium further supplemented with sodium butyrate (Gibco). At 48 h after the media change, virus-containing supernatant was harvested (80 mL total for eight 10 cm plates) and cell debris was removed by centrifugation and 0.45 μm filtration. Virus was pelleted by ultracentrifugation at 24,500 rpm for 94 min at 4° C. in a Beckman Coulter Optima L90K Ultracentrifuge using a SW 28 rotor and 38.5 mL open-top tubes (Beckman Coulter of Brea, CA). Virus pellets were then resuspended in a combined volume of 300 μL serum-free Dulbecco's Modified Eagle Medium (DMEM) and stored at −80° C. until further use.

Cell line culture and T-cell activation. H9, Jurkat (clone E6-1), Raji, K562, and B16F10 cell lines used for SELEX and binding studies were purchased from ATCC. The H9 4IgB7H3 cell line was generated by transducing 10⁵ H9 cells with 3 μL lentivirus encoding 4IgB7H3 with 40 μg/mL protamine sulfate (AAP Pharmaceuticals of East Schaumburg, IL). Transduced cells were later purified by magnetic activated cell sorting (MACS) using PE anti-human B7H3 antibody (1 μL per 106 cells, 351004, BioLegend) and Anti-PE Microbeads (Miltenyi of Auburn, CA) according to the manufacturer's instructions. All the above cell lines were cultured in complete RPMI comprised of RPMI 1640 medium with L-glutamine (Corning) supplemented with 10% FBS, except for B16F10 cells that were cultured in DMEM (Gibco) supplemented with 10% FBS and 1× penicillin-streptomycin (Gibco). Human peripheral blood mononuclear cells (PBMCs) were isolated from TRIMA LRS chambers (Bloodworks Northwest of Seattle, WA) using Ficoll-Paque density gradient centrifugation (GE of Cambridge, MA).

Cell-SELEX. The SELEX procedure was adapted from reported methods (Zumrut et al., (2016) Nucleic Acid Ther, 26, 190-8; Sefah, supra). Conditions used in the individual rounds of SELEX are summarized in Table 2. Briefly, in round 1, 10 nmol of the initial ssDNA library (10¹⁵-10¹⁶ individual sequences) was incubated with 10⁶ H9 4IgB7H3 cells for 1 h at 4° C. in 700 μL wash buffer containing 0.1% BSA and 0.1 mg/mL tRNA. After washing, bound aptamers were extracted by heating cells at 95° C. for 10 min in 500 μL molecular-grade H2O (Corning) and cell debris was removed by centrifugation. Extracted aptamers were amplified with FAM-labeled forward and biotin-labeled reverse primers by PCR using Phusion High-Fidelity DNA Polymerase (NEB) with the following conditions: a 30 s hot start at 98° C., 10-18 cycles of 10 s at 98° C., 30 s at 56° C., and 30 s at 72° C., and lastly a 60 s final extension at 72° C. The resulting double-stranded DNA (dsDNA) product was separated from the PCR solution using High Capacity Streptavidin Agarose Resin (Thermo Fisher of Waltham, MA), and FAM-labeled ssDNA was eluted from the resin with 0.2 M NaOH. The ssDNA was de-salted using a NAP-5 column (GE Healthcare of Chicago, IL) and dried on a Savant ISS110 SpeedVac Concentrator (Thermo Fisher) for the next round of SELEX.

TABLE 2
Experimental conditions for cell-SELEX.

| | | | | | | Salmon | | | | | |
|---|---|---|---|---|---|---|---|---|---|---|---|
| | | | | | Yeast | Sperm | | (+) | | | (−) |
| | DNA | (+) | BSA | FBS | tRNA | DNA | Poly(I:C) | Time | | (−) | Time |
| R | (μM) | Selection | (%) | (%) | (0.1 mg/mL) | (0.1 mg/mL) | (0.1 mg/mL) | (min) | Washes | Selection | (min) |
| 1 | 14 | 10⁷ H9 | 0.1 | 0 | Yes | No | No | 60 | 3x, 30 s | None | None |
| | | 4IgB7H3 | | | | | | | | | |
| 2 | 0.5 | 10⁷ H9 | 0.1 | 0 | Yes | No | No | 60 | 3x, 3 min | 10⁷ H9 | 60 |
| | | 4IgB7H3 | | | | | | | | Parental | |
| 3 | 0.5 | 5 × 10⁶ H9 | 0.5 | 0 | Yes | Yes | No | 45 | 3x, 5 min | 2 × 10⁷ H9 | 60 |
| | | 4IgB7H3 | | | | | | | | Parental | |
| 4 | 0.5 | 4 × 10⁶ H9 | 1 | 0 | Yes | Yes | No | 30 | 4x, 5 min | 2x, 2 × 10⁷ | 30 |
| | | 4IgB7H3 | | | | | | | | H9 | each |
| | | | | | | | | | | Parental | |
| 5 | 0.25 | 2 × 10⁶ H9 | 1 | 0 | Yes | Yes | No | 30 | 5x, 5 min | 4x, 2 × 10⁷ | 30 |
| | | 4IgB7H3 | | | | | | | | H9 | each |
| | | | | | | | | | | Parental | |
| 6 | 0.25 | 2 × 10⁶ H9 | 0 | 2.5 | Yes | Yes | Yes | 30 | 6x, 5 min | 6x, 2 × 10⁷ | 20 |
| | | 4IgB7H3 | | | | | | | | H9 | each |
| | | | | | | | | | | Parental | |
| 7 | 0.1 | 2 × 10⁶ H9 | 0 | 5 | Yes | Yes | Yes | 30 | 6x, 5 min | 100 nM | 30 |
| | | 4IgB7H3 | | | | | | | | B7H3 | |
| | | | | | | | | | | Antibody | |

Table 2 lists the experimental conditions used in rounds I 1-7 of cell-SELEX.

For subsequent rounds of SELEX, the ssDNA concentration, H9 4IgB7H3 cell numbers, and incubation time were slowly decreased during positive selection whereas the concentration of BSA was increased or swapped with FBS. At the same time, additional anionic competitors were introduced for positive selection in later rounds such as salmon sperm DNA (Invitrogen) and poly(I:C) (InvivoGen of San Diego, CA), and the number of washes after binding was gradually extended. After positive selection in rounds 2-6, bound aptamers were extracted by heating positive selection cells at 95° C. for 10 min in 400 μL wash buffer with 0.1% BSA and 0.1 mg/mL tRNA, and cell debris was removed by centrifugation. The extracted aptamers were placed on ice for re-folding before undergoing negative selection with H9 parental cells, whereafter unbound ssDNA was PCR amplified to generate ssDNA pools for use in the next round of SELEX. Starting in round 4, successive negative selections were performed and gradually increased to maximize removal of non-selective binders. In round 7, instead of negative selection, antibody competition was conducted after positive selection by incubating aptamer bound H9 4IgB7H3 cells with 100 nM APC anti-human CD276 (1:13 dilution, BioLegend). The eluted ssDNA from this round was used for NGS.

NGS and analysis. The ssDNA pool from round 7 of cell-SELEX was PCR amplified with the barcoded primers listed in Table 3, purified to remove PCR reagents (Qiagen), and then sequenced using the MiSeq Reagent Kit v2 (300 cycles) and MiSeq System (Illumina of San Diego, CA) according to the manufacturer's instructions. Exported FASTA files were analyzed with FASTAptamer v1.0.3 (Alam et al., (2015) Mol Ther Nucleic Acids, 4, e230); specifically, FASTAptamer-Count was used to determine the rank and reads per million for each sequence identified (Table 4). For the top 50 sequences in round 7, phylogenetic trees were generated with FigTree v1.4.4 (http://tree.bio.ed.ac.uk/software/figtree/) and motifs were discovered with MEME Suite v5.3.0 (Bailey et al., (2009) Nucleic Acids Res, 37, W202-W208). Minimum free energy structures of select aptamer sequences were predicted with the NUPACK web application (Zadeh et al., (2011) J Comput Chem, 32, 170-3).

TABLE 3
Next generation sequencing (NGS) primers.

| Primer | | |
|---|---|---|
| Name | Sequence | Barcode |
| Aptamer F | AATGATACGGCGACCACCGAGATCTACACC | |
| | GAGGAGATACCACTAAGCCACCGTGTCCA | |
| | (SEQ ID NO: 11) | |
| Aptamer_ | CAAGCAGAAGACGGCATACGAGAT*GAATG | |
| R_80 | GAC*ACAGACCGTCGATCCAGAGTGACGCA | |
| | GCA | |
| | (SEQ ID NO: 12) | |

Table 3 lists the primers used for NGS of round 7 pools in the cell-SELEX. Complementary barcode sequence is indicated by asterisks. The complementary barcode sequence is complementary to the barcode in the third column.

TABLE 4
Top 50 round 7 (R7) aptamer sequences.

| R7 | % | | |
|---|---|---|---|
| Rank | Representation | Motif | Sequence |
| 1 | 41.61 | 1 | AACCTGACCTCCTTACTAGATGCAACCCGACTACTAACGTC |
| | | | GTAAGAGAGCC (SEQ ID NO: 13) |
| 2 | 23.77 | 2 | TAGTCAAGGCGATAGACGAAAAACATTGTTTTCCTGGAAGG |
| | | | CCCAGAATCTG (SEQ ID NO: 14) |
| 3 | 6.02 | 4 | CGCAGCAGCGTAAAGGGGGTGTTTGTGCGGTGTGGAGTGC |
| | | | GCGTGCTGCTGC (SEQ ID NO: 15) |
| 4 | 0.83 | 2 | TAGTCAAGGCGATAGACGAAAAACATTGTTTTCCTGGAAGG |
| | | | CCCGGAATCTG (SEQ ID NO: 16) |
| 5 | 0.78 | | GGTTGCTGTACCTACAATTGACAATAAAAAATCTAGCGGGC |
| | | | TTAGAGACAGT (SEQ ID NO: 17) |
| 6 | 0.57 | 3 | AGGCGCTAGACGCAAACACTTAAGCACCATGAGTGCCAAG |
| | | | TTTTCCTAATGG (SEQ ID NO: 18) |
| 7 | 0.39 | 2 | ATAAGTACGCAGTCAAGGCGATAGACGAAAAGAACACTTT |
| | | | CCCGGAAGGGCC (SEQ ID NO: 19) |
| 8 | 0.29 | 2 | ATAGTCAAGGOGATAGACGAAAAACATTGTTTTCCTGGAAG |
| | | | GCCCAGAATCG (SEQ ID NO: 20) |
| 9 | 0.21 | | GCGGAACCGAAGATTTGGCCAAAAAAATCAATCGGGCTTT |
| | | | GGCTCACCCCGT (SEQ ID NO: 21) |
| 10 | 0.13 | 3 | GATGCAAATGCTCGAGAGAGGCACGGCGCAAATCTGGTAG |
| | | | ATGAGCAACGTA (SEQ ID NO: 22) |
| 11 | 0.13 | 1 | AGCCTGACCTCCTTACTAGATGCAACCCGACTACTAACGTC |
| | | | GTAAGAGAGCC (SEQ ID NO: 23) |
| 12 | 0.12 | 1 | AACCTGACCTCCTTACTAGATGCAACCCGACTACTAACGTC |
| | | | GTAAGAGAGTC (SEQ ID NO: 24) |
| 13 | 0.11 | 1 | AACCTGACCTCCTTACTAGATGCAGCCCGACTACTAACGTC |
| | | | GTAAGAGAGCC (SEQ ID NO: 25) |
| 14 | 0.11 | 5 | GCGCAAGTATGGACTATCCCAACAAGGCGTTAGACGTAAAT |
| | | | GAATTATTTCC (SEQ ID NO: 26) |
| 15 | 0.10 | 3 | TCACATCTCGGCAGATGCAAATGCCTGGCCAGGAACCCGAA |
| | | | ACCTGTGATAG (SEQ ID NO: 27) |
| 16 | 0.09 | 3 | CGCAGCAACCCAAACCACCAGCCGGGGATGCAAACACCGC |
| | | | ACAGGGAACGGC (SEQ ID NO: 28) |
| 17 | 0.09 | | AGCTGTAGGTGTGCGGTTAACTCCGTGCTAGATAGGAGGTA |
| | | | TCTCCACAGGG (SEQ ID NO: 29) |
| 18 | 0.09 | 5 | CGCAGCAATCACAGGGCTACAAGGTGCTAAACGTAAACTA |
| | | | GCAAGAGAACTA (SEQ ID NO: 30) |
| 19 | 0.07 | 5 | GAGTAGGAAGAGTAACCAACAAGGCGGTAGACGTAAAAAG |
| | | | ATCAATTTTCCG (SEQ ID NO: 31) |
| 20 | 0.07 | 3 | GACGTTGATTAGGOGGATGCAAACGCACGGTCGTGGTACCA |
| | | | AAACGCACTGC (SEQ ID NO: 32) |
| 21 | 0.07 | 3 | GAAGAGAGTCOGGATGCAAACGCCTGACTAGGATAGACGA |
| | | | TGGTTCTGTGTA (SEQ ID NO: 33) |
| 22 | 0.06 | | AGGTGCTAAACGCAAACCTACCTCTGCTGAGGGCAAGGTGT |
| | | | TTCCTACGGTG (SEQ ID NO: 34) |
| 23 | 0.06 | | ACGCAGCAAGCGGTTTTCGGGTTCGGGTCTGGGGGTTGGGT |
| | | | TGTCGGCACTA (SEQ ID NO: 35) |
| 24 | 0.05 | 3 | GTGACACGTTCCAAATCCACCAACATOGGATGCAAACGCCA |
| | | | GACATGGGTAA (SEQ ID NO: 36) |
| 25 | 0.05 | 1 | AACCTGACCTCCTTACTAGATGCAACCCGACTACTAACGTC |
| | | | GTAAGAGATCC (SEQ ID NO: 37) |
| 26 | 0.05 | 4 | CGCAGCAGCTTAAAGGGGGTGTTTGTGCGGTGTGGAGTGCG |
| | | | CGTGCTGCTGC (SEQ ID NO: 38) |
| 27 | 0.04 | 1 | AACCTGACCTCCTTGCTAGATGCAACCCGACTACTAACGTC |
| | | | GTAAGAGAGCC (SEQ ID NO: 39) |
| 28 | 0.04 | 3 | AAGCTTTATTGGGGATGCAAACACCGAACAGGGAACAATA |
| | | | GATTCCAGAGCA (SEQ ID NO: 40) |
| 29 | 0.04 | 1 | AACCTGACCTCCTTACTAGATGCAACCCGACTGCTAACGTC |
| | | | GTAAGAGAGCC (SEQ ID NO: 41) |
| 30 | 0.04 | 2 | CGGCCCCAAGCATCAAGGCGTTAGACGAAACCCAGGGTTC |
| | | | ACGTAGTGTAAT (SEQ ID NO: 42) |
| 31 | 0.04 | 3 | CGACGTGGTTGACGGATGCAAACGCCTGACAAGGGAATCA |
| | | | ATAGTGGTGTAA (SEQ ID NO: 43) |
| 32 | 0.04 | 3 | TTCCAGACAGGGGGGATGCAAACACATGATAATGGGACCA |
| | | | TGGTTGGAAATC (SEQ ID NO: 44) |
| 33 | 0.04 | 1 | AACCCGACCTCCTTACTAGATGCAACCCGACTACTAACGTC |
| | | | GTAAGAGAGCC (SEQ ID NO: 45) |
| 34 | 0.04 | 1 | AACCTGACCTCCTTACTAGATGCATCCCGACTACTAACGTC |
| | | | GTAAGAGAGCC (SEQ ID NO: 46) |
| 35 | 0.04 | 3 | GATGCAAATGCCTGACGAGGGTGACAGCGAAACTTTGGTAT |
| | | | ATAAGCACCTC (SEQ ID NO: 47) |
| 36 | 0.03 | 3 | GCAAGATGCAACTGATGACTATCTAGGTGGAAGCTAAGTTT |
| | | | CACCGTAATGC (SEQ ID NO: 48) |
| 37 | 0.03 | 3 | TTATCGGGGATGCAACCAACGAATGTGGTACGGGAATGTCT |
| | | | GATGCTACAAA (SEQ ID NO: 49) |
| 38 | 0.03 | 3 | AGAAATGAGCAACGGATGCAAACGCCCAAACGGGGAACTG |
| | | | CGTATATTTTCT (SEQ ID NO: 50) |
| 39 | 0.03 | 3 | TTGCGTGAAGGGAGATGCAACTAACGATAGTGGTACCTGCA |
| | | | CCCGCACTGCA (SEQ ID NO: 51) |
| 40 | 0.03 | 4 | CGCAGCAGCGTAAAGGGGGTGTTTGTGCGGTGTGGAGTGC |
| | | | GCTTGCTGCTGC (SEQ ID NO: 52) |
| 41 | 0.03 | 3 | CCATCTTGACTTCGGATGCAAACGCTTGACCAAGGGAAAGA |
| | | | CATGATGCGCT (SEQ ID NO: 53) |
| 42 | 0.03 | 3 | AGAAGCTGGACTGAGGGGATGCAACCAGAGAATACGGTAC |
| | | | TCATGTCATTGG (SEQ ID NO: 54) |
| 43 | 0.03 | 3 | GAGAATCGGGGATGCAACCAGCTACGOGGTACGGTTCACA |
| | | | ACTTGTACACAC (SEQ ID NO: 55) |
| 44 | 0.03 | 2 | TAGTCAAGGCGATAGGOGAAAAACATTGTTTTOCTGGAAGG |
| | | | CCCAGAATCTG (SEQ ID NO: 56) |
| 45 | 0.03 | 1 | AACCTGACCTCCTTACTAGATGCACCCCGACTACTAACGTC |
| | | | GTAAGAGAGCC (SEQ ID NO: 57) |
| 46 | 0.03 | 2 | TAGTCAAGGCGATAGACGAAAAACATTGTTTTCCTGGAAGG |
| | | | CCCATAATCTG (SEQ ID NO: 58) |
| 47 | 0.03 | 5 | AAAAACGGCCAAGTCATCAAGGCGATAGACGAAAACGAAT |
| | | | CCCTGATTTCCG (SEQ ID NO: 59) |
| 48 | 0.03 | 5 | AGAACAAGGTGTTAAACGTTATGCCCCAGCATACCATCGAA |
| | | | GAGGTCGGCAA (SEQ ID NO: 60) |
| 49 | 0.03 | 5 | ACAGGTCAAGGCGCTAGACGAAGGAAATCTCCTCCATACG |
| | | | GCAATGTTGATT (SEQ ID NO: 61) |
| 50 | 0.03 | 3 | CAAAAGAGATGGATGCAAACACCTGAAAAGGGCATGTCTG |
| | | | AGAAGTGATATA (SEQ ID NO: 62) |

Table 4 lists the top 50 round 7 (R7) aptamer sequences. Sequences do not include constant regions.

Aptamer binding studies. While suspension H9, Jurkat, Raji, and K562 cells were harvested normally, adherent B16F10 cells were non-enzymatically dissociated with Versene (Gibco) prior to binding studies to prevent possible enzymatic cleavage of the α4β1 integrin that would affect aptamer binding. Once harvested, cells (2×10⁵) were pre-stained with Zombie Violet in DPBS for 15 min at room temperature, washed once with wash buffer supplemented with 1% BSA, and incubated with 100 μL folded FAM-labeled ssDNA pools or FAM/Cy5-labeled individual aptamers for 30 min at 4° C. in binding buffer at the indicated concentrations. For antibody co-staining, antibodies were added to the incubation with aptamers. For competitive binding studies, unique aptamers with different fluorescent labels were co-incubated on cells at different concentrations. Cells were then washed twice with 200 μL wash buffer 1% BSA to remove excess staining reagent. If biotinylated aptamers were used for primary staining, cells received a secondary stain with 100 μL fluorescently labeled streptavidin in wash buffer 1% BSA for 20 min at 4° C. and then washed twice as before. Lastly, stained cells were lightly fixed in 200 μL wash buffer 1% BSA with 0.1-0.5% perfluoroalkoxy alkane (PFA) before immediate analysis via flow cytometry as described above. If required, unstained and single-stained cell controls were used for compensation.

Aptamer pull-down assay for receptor identification. The aptamer pull-down assay from cell membrane extracts was adapted from a previously published method (Shangguan et al., (2008) J Proteome Res, 7, 2133-9). For each group (control, HR7A1, and tJBA8.1), 7.5-9×10⁷ cells (Jurkat or H9 cells) were washed three times with DPBS and lysed for 30 min at 4° C. with end-over-end mixing in 3.3 mL hypotonic buffer comprised of 10 mM Tris-HCl PH 7.5 supplemented with Ethylenediaminetetraacetic acid (EDTA)-free complete Protease Inhibitor Cocktail (Roche) and 1 mM PMSF. The resulting cell membranes were pelleted at 16,000 times gravity (xg) for 15 min at 4° C. and washed three times with 3.3 mL of the same hypotonic buffer to remove released intracellular proteins. The membrane pellet was then resuspended in 1 mL wash buffer containing 1% Triton X-100, protease inhibitors, and 1 mM PMSF to extract and solubilize membrane proteins. The extraction was conducted for 30 min at 4° C. with end-over-end mixing followed by a brief 5-min sonication in an ice water bath. Afterwards, the samples were centrifuged as before and the supernatant containing the extracted proteins was collected and stored at −80° C. until further use.

For pre-clearing, the extracts (1 mL) were thawed and spiked with 100 nM of biotinylated random aptamer from the naïve library (RN) and 0.1 mg/mL yeast tRNA. After incubating for 30 min at 4° C. with end-over-end mixing, 200 μL (2 mg) MyOne Streptavidin C1 Dynabeads (Thermo Fisher) were added to the extracts and incubated for another 15 min at 4° C. to remove protein-RN complexes via magnet. For HR7A1 and tJBA8.1 groups, pre-cleared extracts were then spiked with 100 nM of appropriate biotinylated aptamer and 0.1 mg/mL salmon sperm DNA. After incubating for 30 min at 4° C. with end-over-end mixing, 150 μL (1.5 mg) Streptavidin Dynabeads were added to the binding reaction and incubated for another 15 min at 4° C. to capture aptamer-bound proteins. For the control group, 150 μL (1.5 mg) Streptavidin Dynabeads were instead saturated with 50 nmol biotin and added to the pre-cleared extract for a 30-min incubation at 4° C. Afterwards, the beads for all groups were washed 5 times for 5 min each with 1 mL cold wash buffer containing 0.01% Triton X-100 to remove unbound proteins. The captured proteins were eluted by heating the beads for 15 min a 47° C. in 50 μL Laemmli Sample Buffer (Bio-Rad of Hercules, CA) containing 2.5% 2-mercaptoethanol (Bio-Rad), 4.6M urea (Fisher), and 0.01% Triton X-100 (Sigma of St. Louis, MO). Eluted proteins were stored at −80° C. unless used immediately.

Eluted proteins (25 μL) were loaded onto a Novex WedgeWell 8% Tris-Glycine gel (Invitrogen) and separated by SDS-PAGE. The gel was stained with a Colloidal Blue Staining Kit (Invitrogen) according to the manufacturer's instructions and imaged on a Gel Doc EZ system (Bio-Rad). Enriched protein bands were excised and submitted to the Proteomics division at the Fred Hutchinson Cancer Research Center for processing by tandem mass spectrometry on an OrbiTrap Fusion (Thermo Fisher). The data were searched using Proteome Discoverer 2.2 against a Uniprot Human database that included common contaminants. Results were filtered for high confidence 1% false discovery rate at the peptide level.

siRNA knockdown. Some 2×10⁶ Jurkat cells in logarithmic growth phase were nucleofected with 100 pmol NS or CD29 siRNA listed in Table 5 using the Human T cell Nucleofector Kit (Lonza of Basel, Switzerland) with Program X-001 following the manufacturer's instructions. The cells were analyzed 42 h later for HR7A1 binding and anti-CD29 and anti-CD49d antibody staining by flow cytometry. A CD8.A3 negative aptamer control was also included to subtract off any non-specific aptamer binding to the nucleofected cells before data normalization.

TABLE 5
SiRNA duplexes used for CD29 knockdown.

| Name | Sequence |
|---|---|
| hs.Ri.ITGB1.13.1- | 5′-ACUCUUGUCAGCUAAGGUCACAUTG-3′ |
| SEQ1 | (SEQ ID NO: 63) |
| hs.Ri.ITGB1.13.1- | 5′-CAAUGUGACCUUAGCUGACAAGAGUAA-3′ |
| SEQ2 | (SEQ ID NO: 64) |

Table 5 lists the siRNA duplexes used for CD29 (integrin β1) knockdown.

Bio-layer interferometry. BLI studies were performed on an Octet RED96 instrument (Sartorius of Göttingen, Germany) using streptavidin biosensors (Sartorius) as previously described (Cheng et al. (2022) J Am Chem Soc, 144, 13851-64). Briefly, wash buffer supplemented with 1% BSA, 0.1 mg/mL yeast tRNA, 0.1 mg/mL salmon sperm DNA, and 0.01% Tween-20 (Sigma) was used as the sample buffer for all steps. All steps were performed at 25° C. with sample agitation at 1000 rpm. Biosensors were allowed to equilibrate in buffer for at least 10 min before loading. For sensor loading, 50 nM biotinylated aptamers were loaded until a 0.5 nm threshold before performing rinse and baseline steps in buffer alone for 100 s each. Afterwards, aptamer-loaded sensors were associated with His-tagged human α4β1 (IT1-H52W1, ACROBiosystems of Beijing, China) or His-tagged human α4β7 (IT7-H52W4, ACROBiosystems) at concentrations and times indicated in the figures and captions. Lastly, sensors were transferred to wells containing buffer alone for dissociation. Data analysis was performed with the Octet Data Analysis 9.0 software (Sartorius). Association and dissociation curves were normalized to sensors that received capture ligand alone. Kinetic constants were calculated for datasets with several analyte concentrations by conducting a global fit of the association and dissociation curves to a 1:1 ligand binding model. The quality of each fit was evaluated using R2 and χ2 values, which are listed in Table 6 for the appropriate datasets along with the calculated kinetic constants.

TABLE 6
Bio-layer interferometry (BLI) measured affinity kinetics

| Aptamer | K_{D} (nM) | Kₒₙ (nM⁻¹s⁻¹) × 10⁻⁵ | K_{dis} (s⁻¹) × 10⁻⁴ | Full χ² | Full R² |
|---|---|---|---|---|---|
| HR7A1 | 6.28 (±0.04) | 7.43 (±0.03) | 4.67 (±0.02) | 0.2548 | 0.9980 |
| HR7A1.Tr1 | 7.74 (±0.03) | 5.82 (±0.02) | 4.50 (±0.01) | 0.1694 | 0.9993 |
| HR7A1.Tr2 | 9.89 (±0.05) | 4.16 (±0.01) | 4.12 (±0.01) | 0.1619 | 0.9993 |

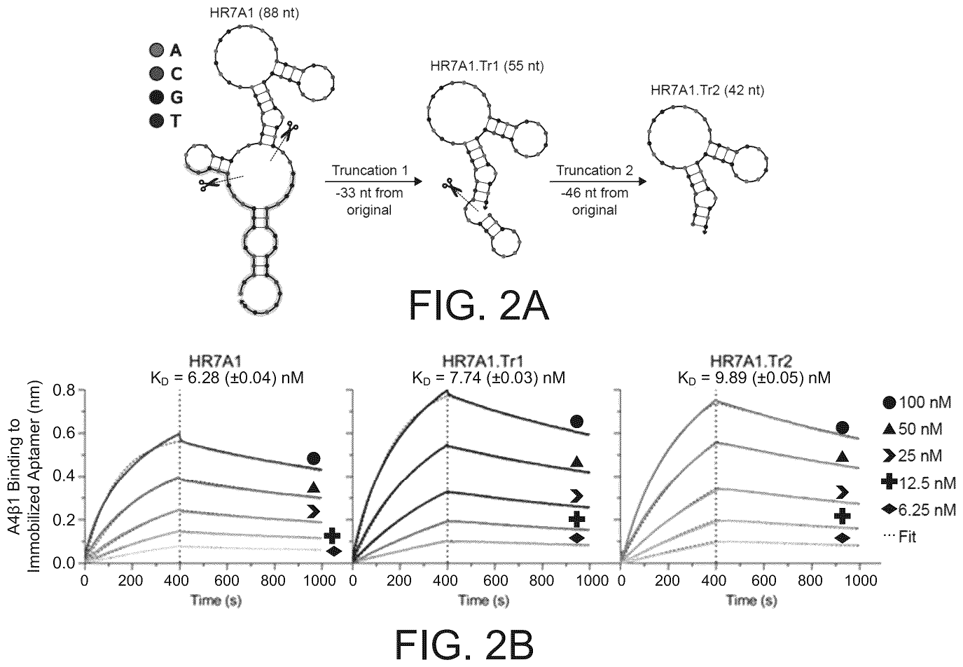

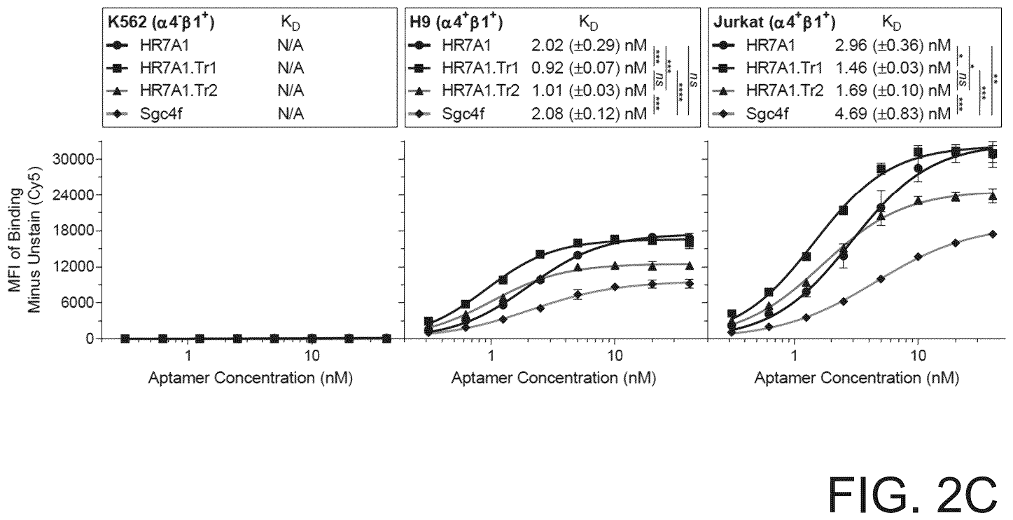

Table 6 lists BLI measured affinity kinetics of immobilized HR7A1, HR7A1.Tr1, and HR7A1.Tr2 aptamer binding to recombinant α4β1 protein. Data are mean±SD, n=5 individual protein concentrations. Values were calculated by performing a global fit of the binding curve data in FIG. 2B to a 1:1 binding model. The ratio between the dissociation rate constant (Kdis) and the association rate constant (Kon) give the equilibrium dissociation constant (KD). The goodness of fit was evaluated by a reduced chi-square (χ2) and a R2 value approaching 1.

Peptide synthesis and purification. The C-terminal biotinylated CS1-b (EILDVPST-b) and SCR (SIELTPVD-b) peptides were synthesized with standard Fmoc-protected amino acids as previously described (Cardle et al., (2021) Journal of Biological Chemistry, 296), except Biotin NovaTag Resin (Novabiochem) was used as the resin support for solid-phase peptide synthesis. Peptides were purified by reverse-phase HPLC with a 1260 Infinity (Agilent), and correct molecular weights of the purified peptides were verified by MALDI-ToF MS on an AutoFlexII (Bruker of Billerica, MA), as previously described (Cardle, supra).

CS1 peptide and VCAM-1 competition. For competition studies involving the CS1 peptide, a Tris-based wash buffer comprised of TBS (Fisher) supplemented with 4.5 g/L glucose, 5.5 mM MgCl², 0.9 mM CaCl², and 10 mM MnCl² was used instead of the normal DPBS-based wash buffer described above. Mn²⁺ has previously been described to support α4β1 binding to the fibronectin CS1 peptide (Masumoto and Hemler. (1993) Journal of Biological Chemistry, 268, 228-234), and as MnCl² precipitates in DPBS, a Tris-based wash buffer was thus required. Peptide stocks were prepared fresh in the Tris-based wash buffer at 20 mM prior to the competition studies and the exact concentration was measured using a QuantTag Biotin Quantification Kit (Vector Labs of Newark, CA). Live/dead and aptamer staining were carried out as described above, except cells were pre-blocked with varying concentrations of the SCR-b or CS1-b peptide in Tris-based wash buffer supplemented with 1% BSA (w/v) and 0.1 mg/mL yeast tRNA for 30 min at room temperature prior to spiking in aptamer and incubating for another 30 min at 4° C. As Tris can neutralize PFA, cells were resuspended in regular wash buffer 1% BSA with 0.5% PFA after washing before analyzing on the flow cytometer. For VCAM-1 competition assays, His-tagged VCAM-1 (VC1-H5224, ACROBiosystems) was co-stained with different concentrations of FAM-labeled aptamers in regular binding buffer as described above, followed by washes and secondary staining with 100 μL APC anti-His tag antibody in wash buffer 1% BSA for 20 min at 4° C. before flow cytometry analysis.

Aptamer serum stability. Normal mouse serum for serum stability studies was prepared in-house. Briefly, whole blood was terminally drawn from mice and allowed to clot for 30 min in BD Microtainer serum-separating tubes (Becton Dickinson). After centrifuging the tubes per the manufacturer's instructions, serum was aliquoted into single-use tubes and stored at −20° C. until needed.

For qualitative assessment of aptamer serum stability, a previously published protocol (Wu et al. (2015) Theranostics, 5, 985-994) was adapted. Briefly, 6 μM of folded FAM-labeled aptamer in wash buffer was mixed 1:1 with normal mouse serum (3 μM aptamer in 50% serum final) and incubated for up to 8 h at 37° C. At 0, 1, 2, 4, 6, and 8 h during the incubation, 10 μl aptamer-serum mixture was removed and frozen at −80° C. to halt nuclease activity until all timepoints were harvested. After the incubation, timepoint samples were then thawed and denatured in 1× loading dye (NEB) containing 4 M urea for 3 min at 70° C. Approximately 12.5 pmol denatured sample was then loaded onto a Novex 15% TBE-urea gel (Invitrogen) and separate by urea-PAGE. FAM-labeled aptamer bands were imaged on a Xenogen IVIS Spectrum (PerkinElmer of Waltham, MA) with 465 nm excitation and 520 nm emission.

For quantitative assessment of functional aptamer serum stability, 1-2 μM of folded Cy5-labeled aptamer in wash buffer was mixed 1:1 with normal mouse serum (0.5-1 μM aptamer in 50% serum final) and incubated for 4, 3, 2, 1, 0.5, and 0 h at 37° C. (staggered timepoints). After the incubation, timepoint samples were placed on ice and diluted to 5-10 nM in binding buffer for staining cells by flow cytometry as described above. Aptamer binding for each timepoint sample was normalized to the 0-h control to assess serum-mediated loss of aptamer functionality.

Aptamer temperature-sensitive binding. Aptamer binding was performed as described above, except aliquots of 2× (10 nM) folded Cy5-labeled aptamer or aptamer-polymer conjugates in binding buffer were pre-equilibrated for 30 min at 4, 20, and 37° C. before mixing 1:1 with Jurkat cells that were similarly pre-equilibrated at the respective temperatures in binding buffer (5 nM aptamer final). The aptamer-cell binding reactions were carried out at the appropriate temperatures for 30 min. Non-specific aptamer binding measured with SCRM controls was subtracted from HR7A1 binding at each temperature before normalizing data to 4° C. binding for assessing temperature-mediated loss of aptamer functionality.

Aptamer plasma circulation half-life. All animal experiments were performed in compliance with the University of Washington (of Seattle, WA) Institutional Animal Care and Use Committee (IACUC) guidelines. Cy5-labeled aptamer or aptamer-polymer conjugates (1 nmol) in wash buffer were each retro-orbitally injected into three C57BL/6 mice (8-12 weeks old). Blood (>10 μL) was drawn at 1-, 5-, 20-, 60-, and 180-min post-injection and collected into pre-weighed tubes with 50 μL DPBS 5 mM EDTA. After blood collection, the tubes were re-weighed to determine the volume of blood collected. The diluted blood was then centrifuged at 1000×g for 10 min at 4° C., and 50 μL of the supernatant was measured for Cy5 fluorescence (Ex: 633/9 nm; Em: 670/20 nm) using an Infinite 200 PRO plate reader (Tecan of Männedorf, Switzerland). The measured fluorescence values at each timepoint were compared to a standard curve of Cy5-labeled aptamer or aptamer-polymer conjugate and normalized by their respective dilution factors to estimate the aptamer concentration in undiluted plasma.

N-(2-Hydroxypropyl) methacrylamide (HPMA) synthesis. 72.1 g of amino-2-propanol (Sigma, 0.96 mol, 2 eq) was dissolved in 1 L of dichloromethane (DCM, Sigma) and chilled over an ice bath. 49.1 g of methacryloyl chloride (Sigma, 0.47 mol, 1 eq) was then added dropwise under vigorous stirring, and the reaction was kept over ice for 30 min after the last drop of methacryloyl chloride was added. The reaction was covered with a glass bung and allowed to warm to room temperature overnight. The next day, an oil/salt solution was removed and discarded from the DCM by filtering the solution through cotton. The DCM was then washed by brine, dried over sodium sulfate, and concentrated at 25° C. by rotary evaporation. Care was taken to not over-concentrate the DCM, which would lead to the HPMA precipitating out. The mixture was then purified by repeated precipitation and recrystallization in acetone (Sigma), with acetone evaporated off in between. A fine, clear, white crystal (20 g) was obtained and characterized by hydrogen-1 NMR (1H NMR).

11-azido-3,6,9-trioxaundecan-1-methacrylamide (AzP3MA) synthesis. 800 mg of 11-azido-3,6,9-trioxaundecan-1-amine (Tokyo Chemical Industries of Tokyo, Japan, 3.7 mmol, 1 eq) and 409 mg of triethylamine (TEA, Sigma, 4.04 mmol, 1.1 eq) were dissolved in 15 mL of anhydrous DCM and chilled over an ice bath. 422 mg of methacryloyl chloride (4.04 mmol, 1.1 eq) was then added dropwise under vigorous stirring. The reaction was kept over ice for 30 min after the last drop of methacryloyl chloride was added. The reaction was covered with a glass bung and allowed to warm to room temperature overnight. The next day, the reaction was quenched by adding 2 mL of methanol and allowing the bubbling to subside. The mixture was diluted to 50 mL with DCM, extracted twice with saturated sodium carbonate and once with brine, and concentrated with a rotary evaporator to obtain a crude yellow oil. The crude mixture was purified with column chromatography (5% v/v methanol:ethyl acetate) to obtain a clear slightly yellow oil (503 mg) and characterized by 1H NMR (vinyl 5.5-6.0 ppm, trioxaundecan 3.5-3.7 ppm).

Biotin-PEG4-CCP synthesis. 606 mg of 4-Cyano-4-(dodecylsulfanylthiocarbonyl)sulfanylpentanoic acid (CCP, Sigma, 1.97 mmol, 1 eq), 1.46 g of tetraethylene glycol (PEG4, Tokyo Chemical Industries, 7.5 mmol, 5 eq) and 27.5 mg of dimethylaminopyridine (DMAP, Sigma, 0.23 mmol, 0.12 eq) was dissolved in 15 mL of DCM and allowed to cool in an ice bath. An excess of PEG4 was used to avoid bi-terminal coupling products. In the meantime, 345 mg of 1-Ethyl-3-(3-dimethylaminopropyl)carbodiimide hydrochloride (EDC, Tokyo Chemical Industries, 1.8 mmol, 0.95 eq) was dissolved in 3 mL DCM. The EDC/DCM was added dropwise over ice while stirring with a stir bar. After addition of the EDC, the reaction was allowed to warm to room temperature and stirred overnight. The reaction was diluted with 150 mL of ethyl acetate, then washed 4× with 0.1M HCl and once with brine. The organic layer was then filtered over sodium sulfate, and rotary evaporated down to obtain a yellow-orange, viscous liquid (900 mg) containing CCP-PEG4. The product was confirmed by TLC and proton NMR.

900 mg of CCP-PEG4 (1.55 mmol, 1 eq), 1.83 g of biotin (Tokyo Chemical Industries, 7.5 mmol, 4.8 eq) and 27.5 mg of DMAP (0.23 mmol, 0.15 eq) was dissolved in 15 mL of DMF (Sigma) and allowed to cool on an ice bath. In the meantime, 345 mg of EDC (Tokyo Chemical Industries, 1.8 mmol, 1.16 eq) was dissolved in 3 mL DMF. The EDC/DMF was added dropwise over ice while stirring with a stir bar. After EDC addition, the reaction was allowed to warm to room temperature and stirred overnight. The reaction was diluted with 150 mL of ethyl acetate, then washed 4× with 0.1M HCl and once with brine. The organic layer was filtered over sodium sulfate, and rotary evaporated to obtain a crude yellow oil. The crude mixture was purified with column chromatography (5% v/v methanol:DCM) to obtain Biotin-PEG4-CCP.

Biotin-PEG4-(HPMA-co-AzP3MA-CCP synthesis and characterization. A 40% wt/wt solution of Methyl-b-cyclodextrin (Fisher of Waltham, MA) in 0.1 M pH 5.0 sodium acetate buffer was first prepared as the reaction solvent to assist in the dissolution of Biotin-PEG4-CCP. With this, a 20 mg/mL solution of Biotin-PEG4-CCP was prepared with 20 minutes of sonication. Thereafter, 200 mg of AzP3MA (0.69 mmol, 37.5 eq) was combined with 498.5 mg of HPMA (3.48 mmol, 187 eq) and 3.1 mL of reaction solvent in a 5 mL pear-shaped flask. Thereafter, 785 uL of Biotin-PEG4-CCP solution (0.019 mmol, 1 eq) and 60.2 uL of a 20 mg/mL solution of VA-044 (0.0038 mmol, 0.2 eq) were added. The solution was purged with argon for 20 min and allowed to react at 50° C. for 26 h. The polymer was purified via dialysis (10 k MWCO RC membrane) in nanopure water for 5 days (d), with a water change every day, and finally lyophilized.

The polymer molar mass was estimated with conversion 1H NMR. Briefly, aliquots of the polymerization reaction at times 0 and 20 h were resuspended into D₂O and analyzed. Overall conversion was determined by quantifying the depletion of the monomeric vinyl peaks (5.5-6.0 ppm) against the solvent peak. The estimation is further refined by NMR of the finished polymer in D2O, taking the ratio of the HPMA peak (4 ppm) and the AzP3MA peak (3.5-3.7 ppm) to obtain the HPMA:AzP3MA ratio. The HPMA and AzP3MA conversions are then individually determined based on overall conversion and the HPMA:AzP3MA ratio.

HPMA-TazP3MA-HR7A1.Tr2.S2E2 synthesis, purification and characterization. For small-scale optimization of aptamer conjugation to polymer, 66.7 μM DBCO-labeled HR7A1.Tr2.S2E2 was reacted with varying amounts of biotin-PEG4-(HPMA-co-AzP3MA)-CCP ranging from 2.22 μM (30:1 aptamer-to-polymer ratio) to 667 μM (1:10 aptamer-to-polymer ratio) in 3 μL DPBS for 24 h at 37° C. on a thermal cycler (Bio-Rad). Afterwards, 250 ng 50 bp DNA ladder (Thermo Scientific), 100 ng unconjugated aptamer, and 100 ng aptamer-polymer conjugates were denatured in 1× loading dye containing 4 M urea for 3 min at 70° C. and separated on a Novex 15% TBE-urea gel by urea-PAGE. The gel was stained with SYBR Gold (1:10000, Invitrogen) in TBE buffer (Thermo Scientific) for 30 min at room temperature and imaged on a Xenogen IVIS Spectrum (PerkinElmer) with 500 nm excitation and 540 nm emission.

For large-scale conjugation of aptamer to polymer, 91 μM DBCO-and Cy5-dual functionalized HR7A1.Tr2.S2E2 was reacted with 91 μM, 30.3 μM, and 18.2 μM HPMA-AzP3MA (1:1, 3:1, and 5:1 aptamer-to-polymer ratio, respectively) in 22-110 μL DPBS for 24 h at 37° C. on a thermal cycler. The aptamer-polymer conjugate was purified and exchanged into wash buffer using a Amicon Ultra-0.5 mL centrifugal filter unit (EMD Millipore) with a nominal molecular weight limit (NMWL) of 50 kDa. The concentration of aptamer in the purified conjugate solution was determined using a NanoDrop UV-Vis spectrophotometer (Thermo Fisher). Cell binding studies with the aptamer-polymer conjugate were carried out as described above based on the measured aptamer concentration.

### Results

Selective therapeutic targeting of T-cell malignancies is notoriously difficult due to the shared lineage between healthy and malignant T cells. Current front-line chemotherapy for these cancers is largely non-specific, resulting in frequent cases of relapsed or refractory disease that have especially grim outcomes. The development of novel targeting approaches for effectively treating T-cell leukemia and lymphoma thus remains a critical goal for the oncology field. This Experimental Example describes a novel DNA aptamer, named HR7A1, that displays single-digit to sub-nanomolar affinity for the heterodimeric integrin α4β1 (also known as VLA-4), a marker associated with chemoresistance and relapse in leukemia patients. After rational truncation of HR7A1 to a minimal binding motif, elevated binding of the aptamer to T-lineage leukemia and lymphoma cell lines over healthy immune cells is demonstrated. Using competition studies, it was found that the aptamer shares an overlapping binding site on the integrin with fibronectin and VCAM-1, which has implications for sensitizing blood cancers to chemotherapy. This Example describes the synthesis of an aptamer-polymer conjugate to address many of these challenges.

This Experimental Example describes a DNA aptamer named HR7A1 that binds to immortalized leukemia and lymphoma cells. Using membrane protein pull-down, the integrin α4β1 (also known as VLA-4) was identified, a marker associated with pediatric r/r lymphoma and lymphocytic leukemia, as a potential target of HR7A1. Aptamer and antibody co-staining, siRNA knockdown, and bio-layer interferometry confirmed aptamer targeting of the human and mouse α4β1 heterodimer and not just one of individual subunits. The 88-nucleotide (nt) HR7A1 sequence was rationally and progressively truncated, generating aptamers as small as 37 nt that retain nanomolar affinity to the α4β1 protein and α4+β1+ cell lines. Of relevance, these truncated HR7A1 aptamers robustly and selectively bind T-lineage malignant cell lines over healthy peripheral immune cells and compete off fibronectin and VCAM-1 binding to the integrin, indicating that they could be used for therapeutic targeting of T-cell leukemias and lymphomas. Modification of one of the truncated aptamers was explored, including nucleotide substitutions and multivalent grafting onto a polymer, to overcome barriers of in vivo translation, laying the foundation for future targeting studies in tumor-bearing mice.

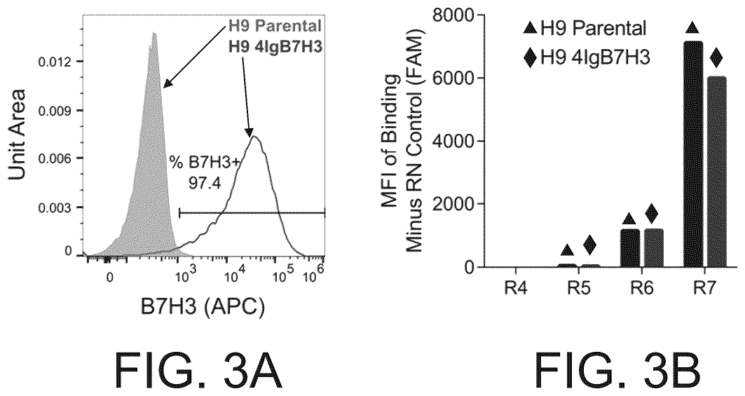

FIGS. 3A, 3B illustrate that aptamer pools from rounds 4-7 (R4-7) of cell-SELEX non-selectively bind both H9 parental and H9 4IgB7H3 cells. (3A) Flow cytometry histograms of extracellular B7H3 expression on H9 parental and H9 4IgB7H3 cells, as measured by antibody staining. Histograms are representative of three independent experiments. (3B) Flow cytometry binding of 250 nM aptamer pools to H9 parental and H9 4IgB7H3 cells after consecutive rounds of SELEX, as measured by MFI. Binding of a random aptamer from the naïve library (RN) at the same concentration was subtracted from values to report specific binding. The bars graphs are representative of one independent experiment. APC, allophycocyanin; FAM, 6-carboxyfluorescein.

Discovery of HR7A1, a leukemia/lymphoma binding aptamer, by cell-SELEX. Cell-SELEX was performed on H9 T-lymphoma cells that were lentivirally transduced to express the predominant isoform of B7H3, 4IgB7H3 (>97% expression, FIG. 3A). H9 cells were chosen due to being a readily available suspension cell line in the lab, eliminating the need for enzymatic dissociation during culturing that can undesirably cleave target extracellular proteins. An initial round of positive selection was conducted against these H9 4IgB7H3 cells using a single-stranded DNA (ssDNA) library with a theoretical 10¹⁵-10¹⁶ unique sequences. Afterwards, the selected aptamer pool underwent multiple rounds of positive and negative selection against H9 4IgB7H3 cells and H9 parental cells, respectively. Selection pressure was increased by decreasing DNA concentration and the number of positive selection cells, switching BSA for FBS, adding additional anion competitors such as salmon sperm DNA and poly(I:C), decreasing positive selection incubation time, increasing washes, and implementing multiple sequential negative selections in a single round, as summarized in Table 2. The enrichment of cell-binding aptamers was monitored by flow cytometry starting after round 4, and non-selective binding to both H9+4IgB7H3 cells and H9 parental cells was observed after round 6 (FIG. 3B). To enrich B7H3 binders from the non-selective pool, ligand-guided selection (LIGS) in round 7 was attempted, which uses antibody competition after the positive selection step to outcompete and elute antigen-specific aptamers (Zumrut, supra). Indeed, a greater hit-rate with SELEX when reversing the order of positive and negative selection has been reported (Yang et al., (2022) Anal Chem, 94, 7278-85).

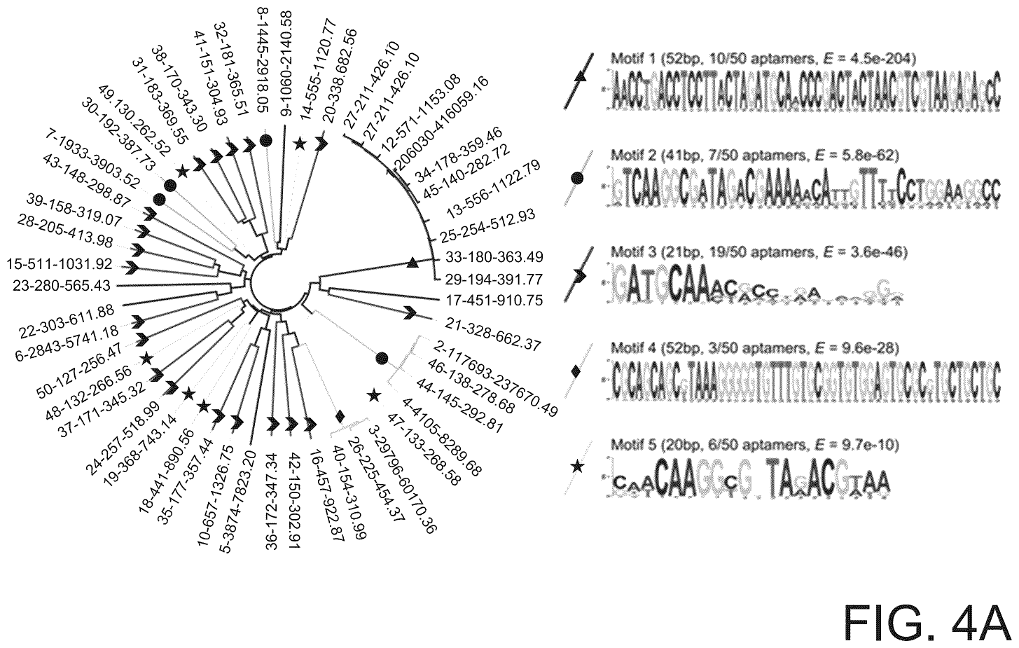

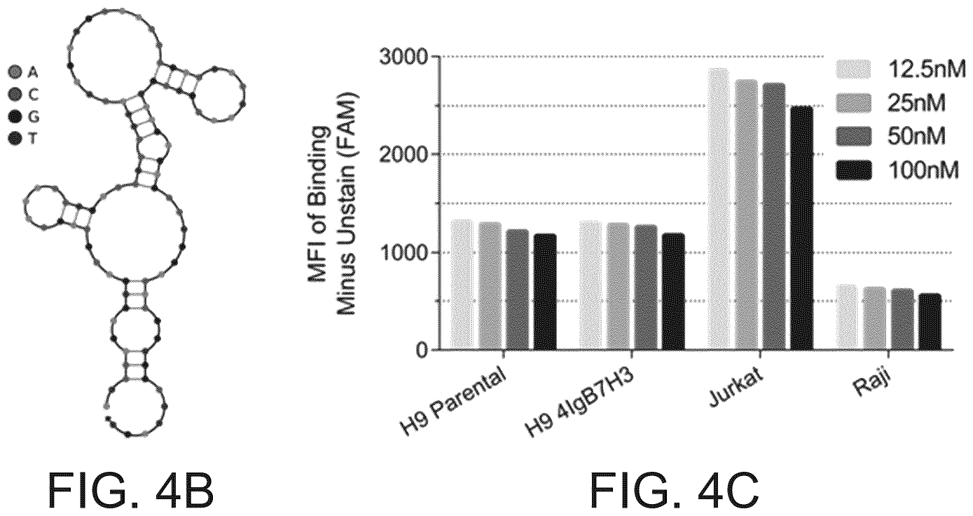

FIGS. 4A-4C illustrate the identification of HR7A1, a highly enriched aptamer that broadly binds lymphoma and lymphocytic leukemia cells. (4A) Phylogenetic tree of the top 50 aptamers from round 7 of cell-SELEX with H9 cells and corresponding consensus motifs. Aptamers with different consensus motifs are highlighted by color in the phylogenetic tree. The E-values of the consensus motifs represents their statistical significance. (4B) The minimum free energy structure of the HR7A1 aptamer, predicted using NUPACK (temperature=4° C.; Na+=137 mM; Mg2+=5.5 mM). (4C) Flow cytometry binding of FAM-labeled HR7A1 to H9 parental, H9 4IgB7H3, Jurkat, and Raji cells at different concentrations, as determined by median fluorescence intensity (MFI). The bars graphs are representative of n=1 independent experiment. FAM, 6-carboxyfluorescein.

Nonetheless, next generation sequencing (NGS) of the round 7 pool using the primers detailed in Table 3 identified H9-binding aptamers of potential interest. FASTAptamer was used to analyze the sequencing data (Alam, supra), and phylogenetic trees and consensus motifs of the top 50 aptamers were generated using FigTree and MEME software, respectively (FIG. 4A) (Caroli et al., (2015) Bioinformatics, 32, 161-164; Bailey, supra). Table 4 shows the top 50 aptamers identified from round 7 in order of prevalence with predicted motifs. Top aptamer sequences collapsed into five motifs, with Motifs 1, 2, and 4 spanning at least 40 nt of the 52-nt random region and containing the rank 1, 2, and 3 aptamers, respectively. In contrast, motifs 3 and 5 were smaller (20 nt), contained lower ranking aptamers, and their respective aptamer sequences clustered less closely on the phylogenetic trees with each other. Surprisingly, Motifs 2 and 4 corresponded to aptamers previously identified by SELEX with different selection cells (Motifs 2 and 4) and starting libraries (Motif 4). Motif 2 represents an aptamer that binds to CD62L (unpublished), and Motif 4 represents the published tJBA8.1 aptamer that binds the transferrin receptor (Cheng, J Am Chem Soc, supra). Motif 1 was a unique motif that had not previously observed; importantly, it was the most statistically significant motif of the five, encompassing the whole 52-nt random region and containing 10 of the top 50 aptamers with 96-98% sequence identity. The rank 1 aptamer from this motif, which was named H9 SELEX Round 7 Aptamer 1 (HR7A1), represented 41.6% of the whole round 7 sequence pool, demonstrating excellent enrichment. The minimum free energy (MFE) structure of HR7A1, predicted by NUPACK (Zadeh, supra) also conformed well to the library design, with the binding motif residing on top of a stem formed by two flanking 18-nt constant regions (FIG. 4B). Due to these properties, the HR7A1 sequence was selected (Table 1) for further characterization.

The binding of fluorescein-labeled HR7A1 was evaluated to four cell lines: H9 parental cells, H9 4IgB7H3 cells, T-leukemia Jurkat cells, and B-lymphoma Raji cells. HR7A1 exhibited broad binding to all these cell lines, with the highest and lowest binding observed on Jurkat and Raji cells, respectively (FIG. 4C). Binding to H9 parental cells was moderate and equivalent to that of the H9 4IgB7H3 cells, indicating that HR7A1 does not bind B7H3. Impressively, HR7A1 binding to all these cells was already saturated at the lowest concentration tested (12.5 nM), demonstrating that HR7A1 has at least single digit nanomolar affinity.

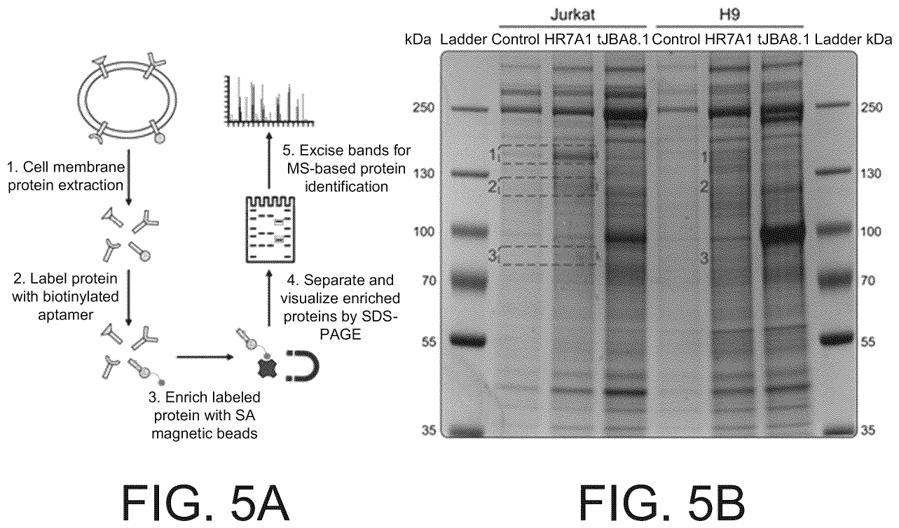

FIGS. 5A, 5B illustrate that integrin α4β1 is identified as a potential target of HR7A1. (5A) Schematic representation of aptamer pull-down procedure for identifying candidate target proteins. (5B) Colloidal blue-stained 8% SDS-PAGE gel of enriched proteins from aptamer pull-down assay with Jurkat and H9 membrane protein extracts. Protein bands that were enriched by HR7A1 relative to the no aptamer and tJBA8.1 controls are marked by numbers, and the dotted boxes represent the bands that were excised for mass spectrometry analysis.

HR7A1 aptamer receptor identification. To identify the target receptor of HR7A1, a pull-down procedure described by Shangguan et al. (FIG. 5A) was used (Shangguan 2008, supra). Briefly, membrane proteins were extracted from Jurkat and H9 cells and incubated with and without biotinylated HR7A1 aptamer, the latter group serving as the negative control. Biotinylated tJBA8.1, which binds to the transferrin receptor, was also included as a positive control. Aptamer-bound proteins were then isolated using streptavidin-coated magnetic beads, and SDS-PAGE was used to separate and visualize enriched protein bands under mild reducing conditions (FIG. 5B). HR7A1 enriched three protein bands relative to the no aptamer and tJBA8.1 controls: an intense band between 130-250 kDa (Band 1), a moderate band between 100-130 kDa (Band 2), and a faint band between 70-100 kDa (Band 3). Encouragingly, these enriched protein bands were found in the membrane protein pull-downs from both Jurkat and H9 cells, although they were noticeably darker for the Jurkat extracts. This tracks with the HR7A1 aptamer's higher binding to Jurkat cells, which points to higher target receptor expression on Jurkat versus H9 cells.

The enriched protein bands from the Jurkat extracts along with corresponding regions of the negative control were excised, digested, and submitted for LC-MS/MS analysis. Ignoring contaminating skin/hair proteins and intracellular proteins, the top protein hit for each excised Jurkat band is shown in Table 7. The integrin α4 (CD49d) was the first ranked candidate from Bands 1 and 3 whereas the integrin β1 (CD29) was the first ranked candidate from Band 2, with minimal representation of these proteins in the corresponding control bands. The presence of multiple integrin α4 bands is consistent with reports that the protein can be expressed fully intact (150 kDa) or as two cleaved, non-covalently associated fragments (70-80 kDa) (Teixidó et al., (1992) Journal of Biological Chemistry, 267, 1786-1791). Together, the integrins α4 and β1 partner to form the α4β1 (CD49d/CD29) heterodimer, also known as very late antigen-4 (VLA-4), which is expressed on a variety of immune cells and interacts with VCAM-1 and fibronectin for cell adhesion and migration during hematopoiesis and inflammation (Imai et al., (2010) Int J Hematol, 91, 569-575). Of relevance to cancer targeting, the integrin α4β1 is overexpressed o many leukemias and lymphomas, and its expression is associated with chemotherapy refractory disease due to the integrin's interaction with stromal cells that promotes cancer cell survival and drug resistance via PI3/AKT/Bcl2 signaling (Matsunaga et al. (2003) Nat Med, 9, 1158-1165; Kurtova et al., (2009) Blood, 113, 4604-4613; Shishido et al., (2014) Front Oncol, 4). Similarly, the expression of integrin α4β1 is also an adverse risk factor in childhood acute lymphoblastic leukemia (ALL) at first relapse (Shalapour et al., (2011) Haematologica, 96, 1627-1635). The tumor-associated expression of this integrin has thus prompted research efforts to target α4β1 for anti-cancer therapy (Choi et al., (2018) Journal of Nuclear Medicine, 59, 1843-1849; Hsieh et al., (2013) Blood, 121, 1814-1818). On account of this and the fact that the expression profile of α4β1 closely resembles the binding profile of the HR7A1 aptamer, this integrin was focused on for further binding validation.

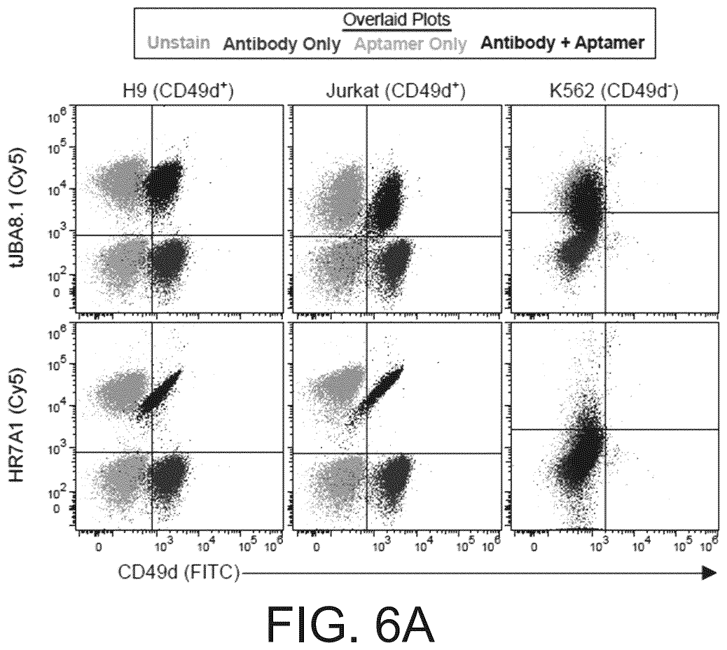

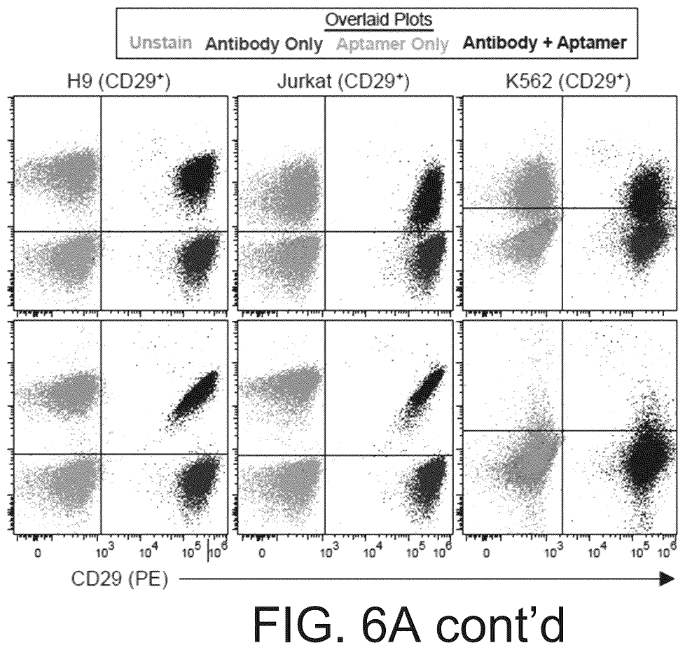

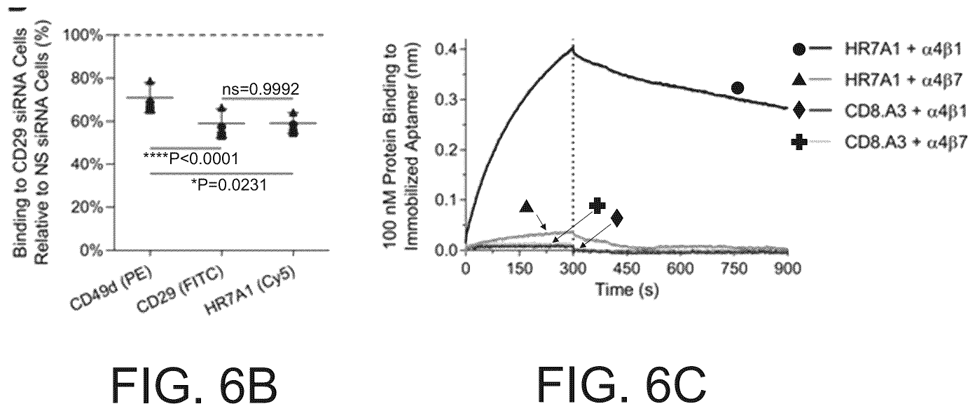

FIGS. 6A-6C illustrate that HR7A1 interacts with both CD49d (α4) and CD29 (β1) for selective recognition of α4β1. (6A) Overlaid flow cytometry plots of 10 nM Cy5-labeled tJBA8.1 and HR7A1 aptamer co-staining with FITC-labeled anti-CD49d and PE-labeled anti-CD29 antibodies on H9, Jurkat, and K562 cells. Plots are representative of n=1 independent experiment. (6B) Flow cytometry analysis of FITC-labeled anti-CD49d antibody, PE-labeled anti-CD29 antibody, and 20 nM Cy5-labeled HR7A1 binding to Jurkat cells, 42 h after nucleofection with a CD29 siRNA duplex. Dashed horizontal line represents binding to non-specific (NS) siRNA-treated controls to which the CD29 siRNA data points were normalized. Horizontal lines and error bars represent mean±SD; n=3 independent experiment. ns>0.05, *P<0.05, ****P<0.0001 (paired one-way ANOVA with Tukey's test). (6C) Association and dissociation kinetics of 100 nM α4β1 and α4β7 binding to biotinylated HR7A1 and CD8.A3 aptamers immobilized on streptavidin biosensors by BLI. The association phase is illustrated from 0-300 s, and the dissociation phase is illustrated from 300-900 s (separated by the vertical dotted line). Data are representative of n=1 independent experiment with one individual concentration of proteins. Cy5, cyanine 5; FITC, fluorescein isothiocyanate; PE, phycoerythrin.

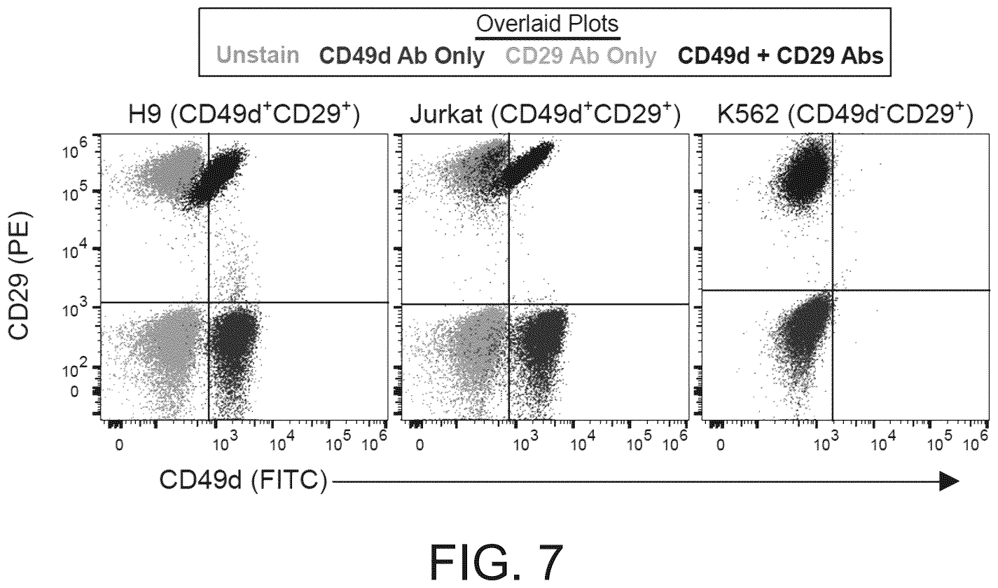

FIG. 7 illustrates that CD49d and CD29 expression on α4+β1+ cell lines correlate with each other. Overlaid flow cytometry plots of FITC-labeled anti-CD49d antibody (CD49d Ab) and PE-labeled anti-CD29 antibody (CD29 Ab) co-staining on H9, Jurkat, and K562 cells. Plots are representative of one independent experiment. FITC, fluorescein isothiocyanate; PE, phycoerythrin.

Validation of HR7A1 binding to α4β1. To confirm the mass spectrometry results, co-staining of the HR7A1 aptamer with anti-CD49d (anti-α4) and anti-CD29 (anti-β1) antibodies was observed on various target cell lines (FIG. 6A), including H9 and Jurkat cells, as well as erythroleukemia K562 cells, which have previously been shown to express α5β1 (CD49e/CD29) but not α4β1 (Bing et al., (2015) Molecular & Cellular Proteomics, 14, 2692-2700; Hemler et al., (1987) Journal of Biological Chemistry, 262, 3300-3309). On H9 and Jurkat cells, HR7A1 aptamer binding correlated strongly with anti-CD49d and anti-CD29 antibody staining, whereas binding of a tJBA8.1 aptamer control did not correlate with antibody staining. The HR7A1 aptamer did not bind to CD49d−CD29+ K562 cells, indicating that HR7A1 is specific for either the integrin α4 alone or the α4β1 dimer. Correlation between anti-CD49d and anti-CD29 antibody staining was also observed (FIG. 7). Consequentially, the correlation between HR7A1 aptamer binding and anti-CD29 antibody staining could be either a direct effect of binding to the α4β1 dimer or an indirect effect of binding to the integrin α4, necessitating further testing.

Accordingly, Jurkat cells were nucleofected with non-specific (NS) and CD29-specific short interfering RNA (siRNA) duplexes (Table 5) and evaluated changes in protein expression and aptamer binding to distinguish if HR7A1 binds the integrin α4 alone or the α4β1 dimer. Interestingly, nucleofection with CD29 siRNA caused not only a 41% reduction in CD29 expression but also a 29% reduction in CD49d expression, indicating that CD29 is required for proper expression of CD49d (FIG. 6B). HR7A1 aptamer binding to CD29 siRNA-nucleofected cells was also reduced and strikingly matched the loss of CD29 expression but not the decrease in CD49d expression. Given that HR7A1 does not bind to CD49d−CD29+ K562 cells, these results indicate that the aptamer specifically binds the α4β1 heterodimer and not just one of the individual subunits.

To further validate the integrin specificity of HR7A1, bio-layer interferometry (BLI) binding of immobilized HR7A1 was performed to recombinant α4β1 and α4β7, the latter of which is an integrin involved in homing of lymphocytes to the gut (Petrovic et al., (2004) Blood, 103, 1542-1547; Kurmaeva et al., (2014) Mucosal Immunol, 7, 1354-1365). Compared to the published CD8-binding A3 aptamer (CD8.A3) that served as a negative control (37), HR7A1 displayed fast and robust binding to α4β1 that dissociated slowly upon removing excess protein (FIG. 6C). In contrast, while HR7A1 did bind α4β7 over the control CD8.A3 aptamer, the resulting binding signal was at least 10-fold lower than it was for α4β1, and α4β7 dissociated quickly from HR7A1 after removal of excess protein. This weak binding to α4β7 presents further evidence that α4 alone is not able to mediate tight HR7A1 binding. Taken together, these assays demonstrate that HR7A1 simultaneously interacts with both the integrin α4 and the integrin β1 to facilitate high-affinity, selective targeting of the α4β1 heterodimer.

FIGS. 2A-2C illustrate that HR7A1 binds α4β1 with single-digit nanomolar affinity and can be significantly truncated without loss of function. (2A) Rational truncation of the HR7A1 sequence to remove stem-forming constant regions (grey), resulting in HR7A1.Tr1 and HR7A1.Tr2. MFE secondary structures for each sequence are shown, predicted using NUPACK (temperature=4° C.; Na+=137 mM; Mg2+=5.5 mM). The dashed lines indicate the sites of truncation. (2B) Association and dissociation kinetics of serially diluted α4β1 binding to biotinylated HR7A1, HR7A1.Tr1, and HR7A1.Tr2 aptamers immobilized on streptavidin biosensors by BLI. The association phase is illustrated from 0-400 s, and the dissociation phase is illustrated from 400-1000 s (separated by the vertical dotted line). KD values were calculated by performing a global fit of the multi-concentration kinetic data to a 1:1 binding model. KD values represent mean±SD; n=5 individual concentrations of protein. (2C) Flow cytometry binding curves of Cy5-labeled HR7A1, HR7A1.Tr1, HR7A1.Tr2, and Sgc4f to K562, H9, and Jurkat cells, as determined by MFI. The curves represent a nonlinear regression assuming one-site specific binding with Hill slope. KD values were calculated by averaging the individual regression values of the independent experiments. Data points and error bars, and KD values, represent mean±SD; n=3 independent experiments. ns>0.05, *P<0.05, **P<0.01, ***P<0.001, ****P<0.0001 (ordinary one-way ANOVA with Tukey's test). Cy5, cyanine 5.

HR7A1 aptamer truncation and binding affinity comparison to other α4β1-binding aptamers. The full-length HR7A1 aptamer contains 88 nt, including two 18-nt flanking constant regions that were used for PCR amplification during SELEX. While constant regions can be important for stabilizing aptamer structure, often they can be omitted or partially truncated without impacting aptamer binding (Wang et al., (2017) Sci Rep, 7, 1-10). By shortening the DNA sequence, aptamer production costs can be reduced even further while increasing synthesis yields, which can enable production of large quantities of aptamers needed for in vivo applications. Accordingly, two truncated versions of the HR7A1 aptamer were synthesized by gradually removing nucleotides from the 5′ and 3′ termini (FIG. 2A). The first truncation (minus 33 nt from the original sequence) removed the majority of the 5′ and 3′ constant regions except for 5 nt at the end of the 5′ constant region that was predicted to form a small hairpin structure with the consensus motif. The second truncation (minus 46 nt from the original sequence) was added onto the first truncation to further remove that small hairpin structure. These truncations yielded the sequences HR7A1.Tr1 and HR7A1.Tr2, respectively (Table 1).

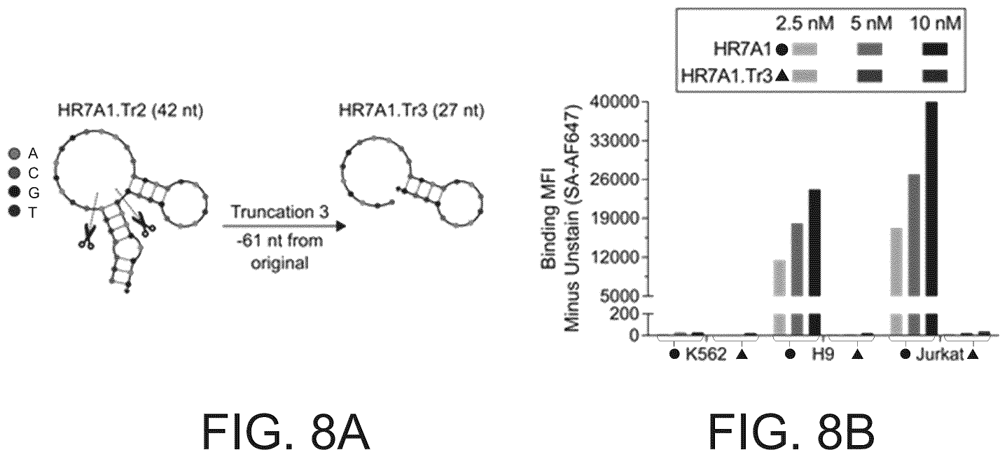

FIGS. 8A, 8B illustrate that HR7A1.Tr3 does not retain binding capabilities of full-length aptamer. (8A) Truncation of the HR7A1.Tr2 sequence to create HR7A1.Tr3. MFE secondary structures for each sequence are shown, predicted using NUPACK (temperature=4° C.; Na+=137 mM; Mg2+=5.5 mM). The dashed lines indicate the sites of truncation. (8B) Flow cytometry binding of biotinylated HR7A1 and HR7A1.Tr3 to K562, H9, and Jurkat cells, as measured by MFI. The bars graphs represent one independent experiment. SA-AF647, streptavidin Alexa Fluor 647.

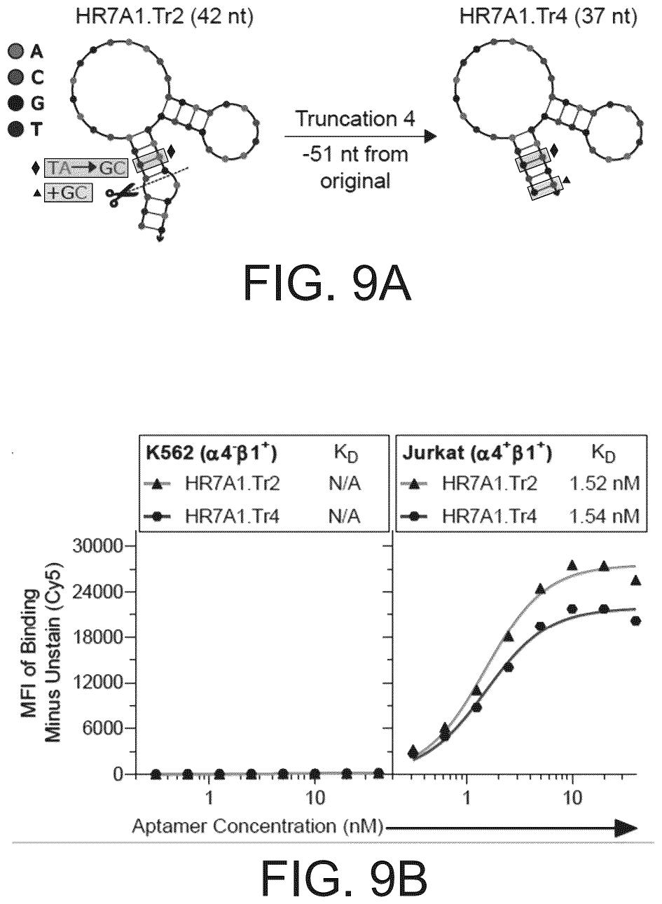

FIGS. 9A, 9B illustrate that HR7A1.Tr4 is the minimal binding motif of HR7A1. (9A) Truncation of the HR7A1.Tr2 sequence to create HR7A1.Tr4. MFE secondary structures for each sequence are shown, predicted using NUPACK (temperature=4° C.; Na+=137 mM; Mg2+=5.5 mM). The dashed lines indicate the sites of truncation. Highlighting denotes GC base pairs that were substituted into (diamond) or appended to (triangle) the truncated stem for added stability. (9B) Flow cytometry binding curves of Cy5-labeled HR7A1.Tr2 and HR7A1.Tr4 to K562 and Jurkat cells, as determined by MFI. The curves represent a nonlinear regression assuming one-site specific binding with Hill slope. Data points are representative of n=1 independent experiment. Cy5, cyanine 5.

The binding kinetics of the full-length HR7A1 aptamer and these truncated variants were compared to recombinant α4β1 by BLI. HR7A1.Tr1 and HR7A1.Tr2 bound to the α4β1 protein with KD values of 7.74±0.03 and 9.89±0.05 nM, respectively, demonstrating negligible loss in binding ability compared to the KD of 6.28±0.04 nM for the full-length HR7A1 aptamer (FIG. 2B and Table 6). Interestingly, both the association rate and dissociation rate of aptamer binding to α4β1 decreased with cumulative truncation, indicating that the truncated variants have slower but also more stable binding to α4β1. The predicted lower stem of HR7A1.Tr2 was truncated, resulting in the 27-nt HR7A1.Tr3 sequence (FIG. 8A and Table 1), but this aptamer failed to bind to Jurkat and H9 cells (FIG. 8B). However, partial truncation of the HR7A1.Tr2 stem that further included stabilizing guanine-cytosine (GC) base pair substitutions/additions did yield a smaller (minus 5 nt) aptamer, named HR7A1.Tr4 (FIG. 9A and Table 1), that retained binding affinity to Jurkat cells (FIG. 9B). In this Example, this sequence represents the minimal binding motif required for high affinity α4β1 recognition.

HR7A1 is likely not the first α4β1-specific aptamer to have been discovered. Sgc4 and its truncated 60-nt Sgc4e and 56-nt Sgc4f sequences are aptamers discovered by the Tan group that reportedly bind the α4 integrin (Shangguan et al., (2006) Proceedings of the National Academy of Sciences, 103, 11838-11843; Bing, supra; Shangguan et al., (2007) ChemBioChem, 8, 603-606). Of relevance to the application of targeting T-cell malignancies, Sgc4 was found to robustly bind T-cell acute lymphoblastic leukemia (T-ALL) cell lines and patient samples over healthy immune cells in normal bone marrow (Shangguan et al., (2007) Clin Chem, 53, 1153-1155). Given this data, it was sought to compare the cell binding capabilities of the HR7A1, HR7A1.Tr1, and HR7A1.Tr2 aptamers against Sgc4f by flow cytometry (Table 7). Like the HR7A1 aptamer, HR7A1.Tr1, HR7A1.Tr2, and Sgc4f did not bind α4−β1+ K562 cells, again showing that β1 alone is not able to facilitate binding of these aptamers. Analyzing binding to H9 cells and Jurkat cells that express α4β1 at different levels, all four aptamers boasted apparent KD values of 0.9-2.1 nM and 1.5-4.7 nM for the respective cells. HR7A1.Tr1 and HR7A1.Tr2 bound H9 and Jurkat cells with two-to three-fold lower KD values than those of HR7A1 and Sgc4f, indicating that the truncations made to HR7A1 provided enhancements in equilibrium (i.e., steady-state) binding affinity at 4° C. Additionally, HR7A1.Tr2 exhibited lower maximum binding MFI values than HR7A1 and HR7A1.Tr1, which were similarly observed with HR7A1.Tr4 when compared to HR7A1.Tr2 in FIG. 9B. This reduced fluorescence may be attributed to Cy5 fluorescence quenching caused by neighboring nucleotides or the closer proximity of the fluorophore to the aptamer-protein binding interface caused by successive truncation (Kretschy et al., (2016) Bioconjug Chem, 27, 840-848). Comparing HR7A1 to Sgc4f, the two aptamers displayed similar apparent binding affinities to H9 cells whereas HR7A1 had slightly higher affinity over Sgc4f towards Jurkat cells.

TABLE 7
Extracellular protein hit identified by mass spectrometry.

| | | | | | | | SEQUEST | |
|---|---|---|---|---|---|---|---|---|
| | Molecular | | Peptide Coverage | | # of Peptides | | HT Score | |
| Band | Weight | Description | Control | HR7A1 | Control | HR7A1 | Control | HR7A1 |
| 1 | >130 kDa & | Integrin | 0% | 34% | 0 | 41 | 0 | 224 |
| | <250 kDa | alpha 4 | | | | | | |
| 2 | >100 kDa & | Integrin | 1% | 35% | 1 | 40 | 1.65 | 311 |
| | <130 kDa | beta 1 | | | | | | |
| 3 | >70 kDa & | Integrin | 0% | 18% | 0 | 17 | 0 | 72 |
| | <100 kDa | alpha 4 | | | | | | |

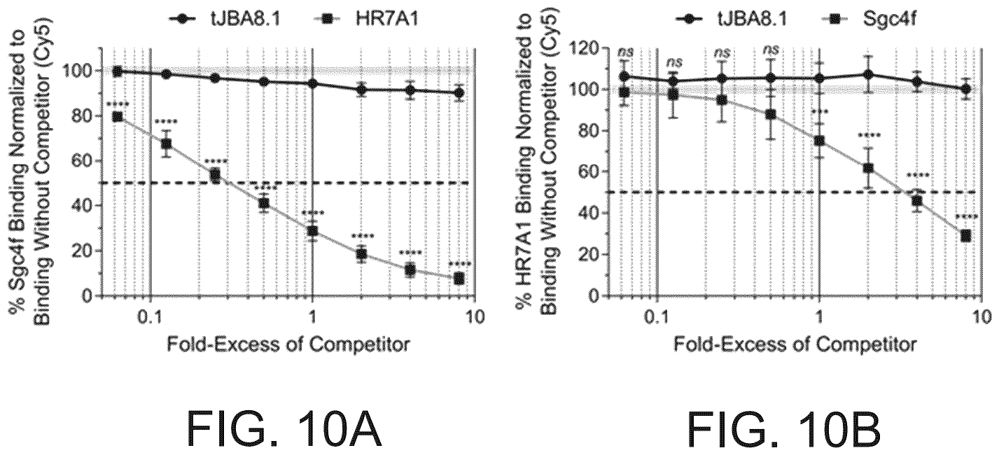

Table 7 includes a summary of the top extracellular protein hit for each excised band identified by mass spectrometry. A higher SEQUEST HT score represents a better identification. Integrin alpha 4: 114.8 kDa (migrates as 150 kDa and 70-80 kDa); Integrin beta 1: 88.4 kDa (migrates as 110-130 kDa). FIGS. 10A, 10B illustrate that HR7A1 outcompetes Sgc4f for binding to α4β1-expressing cells. Competitive binding of (10A) 20 nM Cy5-labeled Sgc4f with varying fold excess of HR7A1 and (10B) 20 nM Cy5-labeled HR7A1 with varying fold excess of Sgc4f to Jurkat cells by flow cytometry. Varying fold excess of TfR1-binding tJBA8.1 was also included in each assay as a negative competitor control. Binding was normalized to aptamer-stained cells without competitor aptamer. Data points and error bars represent mean±SD; n=3 independent experiments. ns>0.05, ***P<0.001, ****P<0.0001 (ordinary two-way ANOVA with Sidak correction). Cy5, cyanine 5.

To assess whether HR7A1 and Sgc4f share a binding site on α4β1, competitive binding studies on Jurkat cells were performed. Increasing concentrations of HR7A1, but not a TfR1-binding tJBA8.1 control aptamer, robustly outcompeted a fixed concentration of Sgc4f for binding to Jurkat cells, demonstrating that the two aptamers share overlapping binding sites on α4β1 (FIG. 10A). Furthermore, the relative affinity of HR7A1 for Jurkat cells appeared to be at least three-fold higher than that of Sgc4f, as only 0.3-fold excess of HR7A1 was required to compete off 50% of Sgc4f binding. The assay was repeated with a fixed concentration of HR7A1 and increasing concentrations of Sgc4f and similarly observed selective competition between the two aptamers, with more than three-fold excess of Sgc4f required to compete off 50% of HR7A1 binding to Jurkat cells (FIG. 10B). Collectively, these results demonstrate that the HR7A1 aptamer family shares an overlapping binding epitope on α4β1 with the Sgc4 aptamer family despite having dissimilar sequences (Table 1). Moreover, the HR7A1 aptamer family binds more tightly to the α4β1protein and with a smaller minimal binding motif than the Sgc4 aptamer family (37-nt HR7A1.Tr4 versus 56-nt Sgc4f). Further studies will be needed to conclusively determine if Sgc4f binds α4 alone as reported or the α4β1 heterodimer.

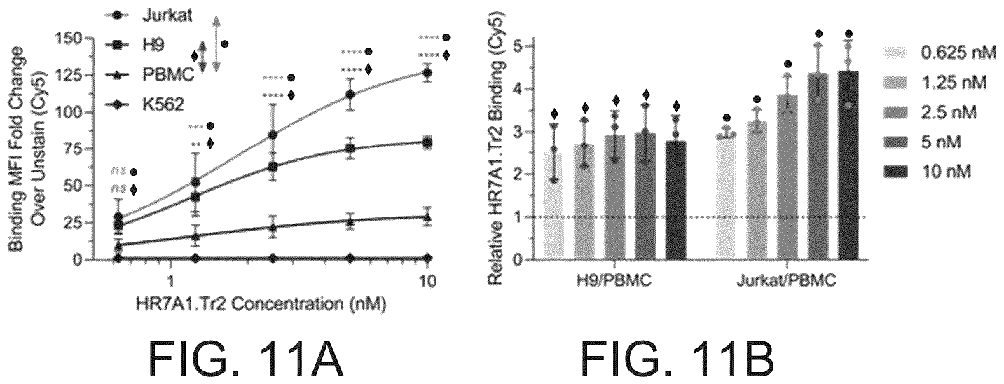

FIGS. 11A, 11B illustrate that HR7A1.Tr2 selectively binds immortalized T-leukemia and T-lymphoma cancer cells over healthy PBMCs. (11A) Flow cytometry binding curves of Cy5-labeled HR7A1.Tr2 to T-leukemia Jurkat cells, T-lymphoma H9 cells, healthy donor PBMCs, and erythroleukemia K562 cells, as determined by MFI fold change over unstained controls. The curves represent a nonlinear regression assuming one-site specific binding with Hill slope. Data points and error bars represent mean±SD; n=3 independent experiments with different PBMC donors. ns>0.05, **P<0.01, ***P<0.001, ****P<0.0001 (circle: significance between aptamer binding to Jurkat cells and PBMCs; diamond: significance between aptamer binding to H9 cells and PBMCs; ordinary two-way ANOVA with Dunnett's test). (11B) Relative binding of Cy5-labeled HR7A1.Tr2 to H9 and Jurkat cells over PBMCs at each of the concentrations tested. Values that fall above the dashed line represent cancer-selective binding over PBMCs. Graph bars and error bars represent mean±SD; n=3 independent experiments with different PBMC donors. Cy5, cyanine 5.

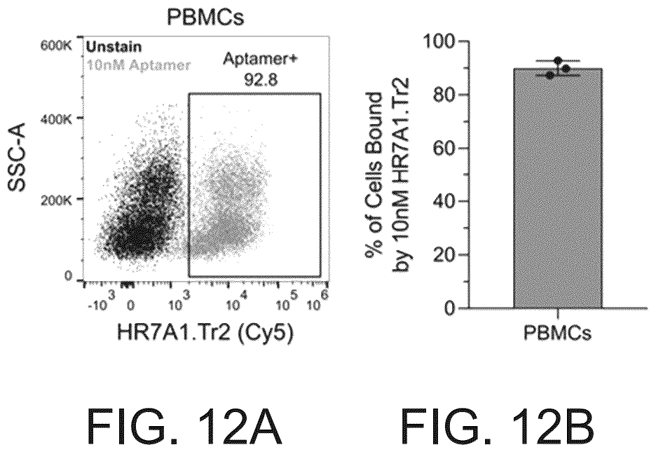

FIGS. 12A, 12B illustrate that HR7A1.Tr2 binds the majority of PBMCs. (12A) Overlaid flow cytometry plot of 0 nM (unstain) and 10 nM Cy5-labeled HR7A1.Tr2 aptamer binding to PBMCs. The plot is representative of n=3 independent experiments with different PBMC donors. (12B) Corresponding percentage of PBMCs labeled by 10 nM Cy5-labeled HR7A1.Tr2 staining. Bar graph and error bars represent mean±SD; n=3 independent experiments with different PBMC donors. Cy5, cyanine 5.

Characterization of cancer-selective properties of HR7A1.Tr2. While the integrin α4β1 is overexpressed in leukemia, it is also expressed by hematopoietic stem cells (HSCs) and healthy peripheral immune cells (Imai, supra), posing a risk of on-target off-tumor toxicities if the integrin is therapeutically targeted. Accordingly, cancer targeting must be sufficiently selective to mitigate these potential side effects. To characterize the on-target off-tumor labeling with the aptamers in vitro, the relative binding of HR7A1.Tr2 at multiple concentrations was compared to healthy donor PBMCs and immortalized Jurkat, H9, and K562 cells. As PBMCs have lower baseline MFI values than immortalized cancer cells on flow cytometry, which can negatively skew raw binding MFIs, aptamer binding was evaluated as a fold change in MFI from each cell's baseline. HR7A1.Tr2 displayed low but specific binding to PBMCs over α4−β1+ K562 cells (FIG. 11A), binding on average 90% PBMCs at 10 nM (FIGS. 12A, 12B). Despite this, aptamer binding to Jurkat and H9 cells was statistically higher at almost all concentrations, labeling these cancer cells 2.5-4.3 times more than PBMCs depending on the concentration (FIG. 11B). While further comparative screening of aptamer binding to CD34+ hematopoietic stem cells (HSCs) and patient-derived leukemia/lymphoma samples is required, these data provide encouraging evidence that HR7A1.Tr2 can selectively target T-lineage leukemia and lymphoma cells over healthy counterparts via α4β1 binding.

Determination of HR7A1.Tr2 binding epitope on α4β1. The α4β1 integrin is known to interact with several ligands, including VCAM-1 expressed by activated vascular endothelium and bone marrow stromal cells (Elices et al., (1990) Cell, 60, 577-584; Oostendorp et al., (1995) Br J Haematol, 91, 275-284) and the highly abundant extracellular matrix (ECM) glycoprotein fibronectin (Garcia-Pardo et al., (1990) The Journal of Immunology, 144, 3361). Importantly, α4β1 binding to VCAM-1 on bone marrow stromal cells has been shown to mediate drug resistance in B- and T-lineage leukemia and lymphoma cells via various signaling pathways (Matsunaga, supra; Kurtova, supra; Mudry, et al., (2000) Blood, 96, 1926-1932; Jacamo et al. (2014) Blood, 123, 2691-2702; Berrazouane et al., (2021) Cancers (Basel), 13, 3512), and blocking this interaction can sensitize these cancer cells to chemotherapy (Shalapour, supra; Hsieh, supra; Berrazouane et al., (2019) Cell Death Dis, 10, 357). Accordingly, an aptamer that blocks α4β1 binding to its natural ligands would be especially valuable for targeted anti-cancer treatment.

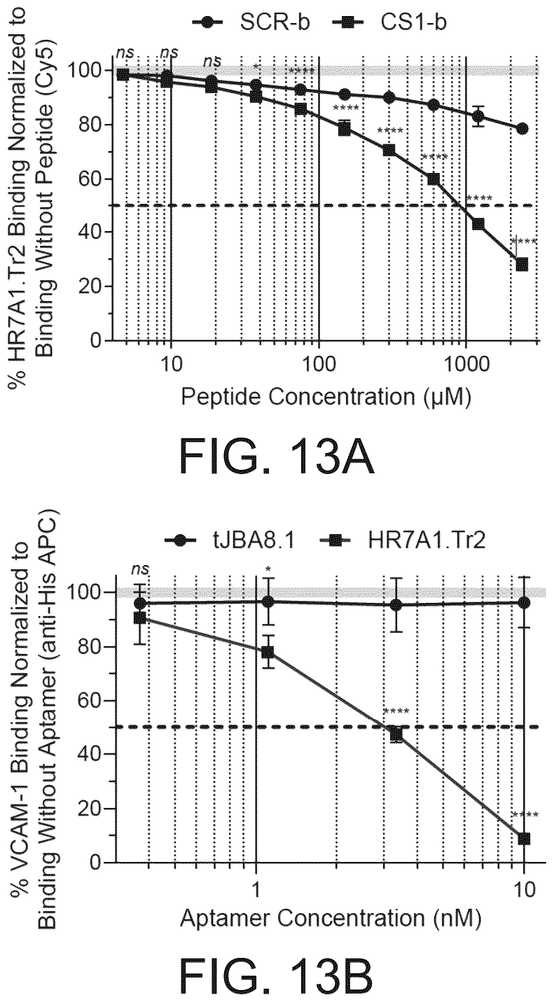

FIGS. 13A, 13B illustrate that HR7A1.Tr2, fibronectin, and VCAM-1 share an overlapping binding epitope on α4β1. (13A and 13B) Competitive binding of (13A) 10 nM Cy5-labeled HR7A1.Tr2 to Jurkat cells pre-incubated with varying concentrations biotinylated fibronectin CS1 peptide (CS1-b) and (13B) 100 nM His-tagged VCAM-1 and varying concentrations of HR7A1.Tr2 to Jurkat cells by flow cytometry. A biotinylated scrambled peptide (SCR-b) and TfR1-binding tJBA8.1 were included in the respective assays as negative competitor controls. Binding was normalized to stained cells without competitor. Data points and error bars represent mean±SD; n=3 independent experiments. ns>0.05, *P<0.05, ****P<0.0001 (ordinary two-way ANOVA with Sidak correction). Cy5, cyanine 5; APC, allophycocyanin.

To evaluate if HR7A1.Tr2 shares an overlapping binding epitope on α4β1 with fibronectin and VCAM-1, we performed competitive binding studies were performed on Jurkat cells similarly to what was done with Sgc4f. For fibronectin competition, a C-terminal biotinylated CS1 peptide (CS1-b) was synthesized that was derived from the type III homology connecting segment (IIICS) of fibronectin that is known to bind α4β1 (Wayner, supra; Komoriya et al., (1991) Journal of Biological Chemistry, 266, 15075-9). Jurkat cells were pre-treated with micromolar-to-millimolar concentrations of CS1-b peptide and evaluated HR7A1.Tr2 binding at a fixed concentration by flow cytometry. Increasing concentrations of CS1-b, but not a biotinylated scrambled peptide control (SCR-b), selectively outcompeted HR7A1.Tr2 binding to Jurkat cells, affirming that HR7A1.Tr2 and fibronectin share an overlapping binding epitope on α4β1 (FIG. 13A). Nearly 1 mM CS1-b was required to block 50% of 10 nM HR7A1.Tr2 binding, which demonstrates the vastly superior binding affinity of HR7A1.Tr2. For VCAM-1, binding of recombinant protein to Jurkat cells was distinguished by flow cytometry, enabling direct assessment of competition. Compared to the TfR1-binding tJBA8.1 control aptamer, which did not inhibit VCAM-1 binding to Jurkat cells during co-staining, HR7A1.Tr2 robustly antagonized VCAM-1 binding (FIG. 13B). Only 3 nM of HR7A1.Tr2 was required to compete off 50% of 100 nM VCAM-1 binding, which is consistent with the reported 40 nM steady state binding affinity of VCAM-1 for α4β1 (Chakraborty et al., (2015) PLoS One, 10).

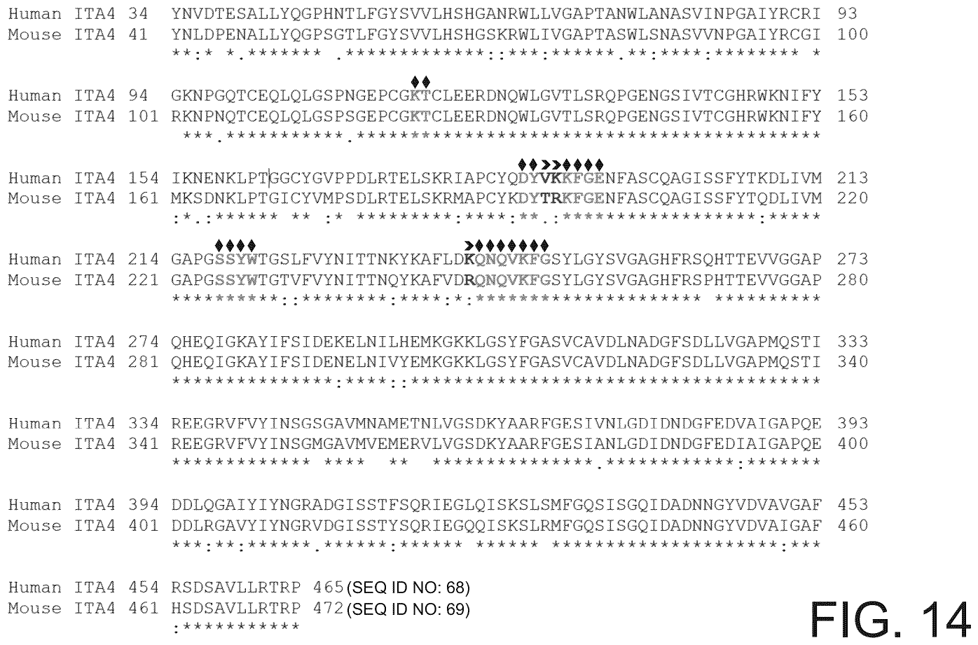

FIG. 14 illustrates that mouse integrin α4 shares high sequence similarity with human α4 in beta-propeller domain. Protein sequence alignment of the human and mouse α4 beta-propeller domains using the Clustal Omega program. Residues marked with a diamond represents the α4 residues predicted to interact with HR7A1.Tr2 that overlap between the human and mouse sequences, whereas residues marked with a chevron represents the α4 residues predicted to interact with HR7A1.Tr2 that do not overlap between the human and mouse sequences.

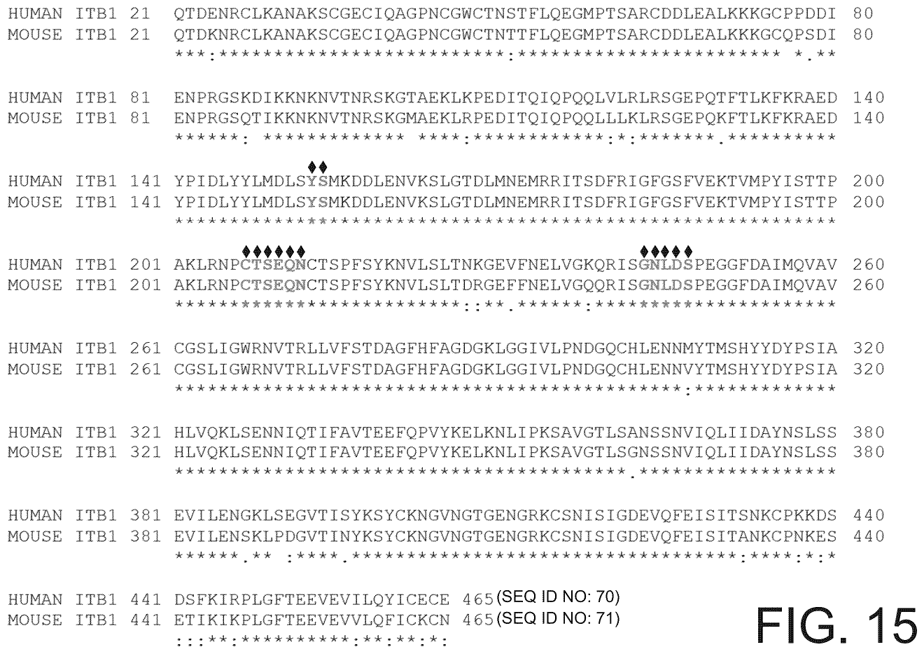

FIG. 15 illustrates that mouse integrin β1 shares high sequence similarity with human β1 in β1 and hybrid domains. Protein sequence alignment of the human and mouse β1 βI and hybrid domains using the Clustal Omega program. Residues marked with a diamond represents the α4 residues predicted to interact with HR7A1.Tr2 that overlap between the human and mouse sequences.

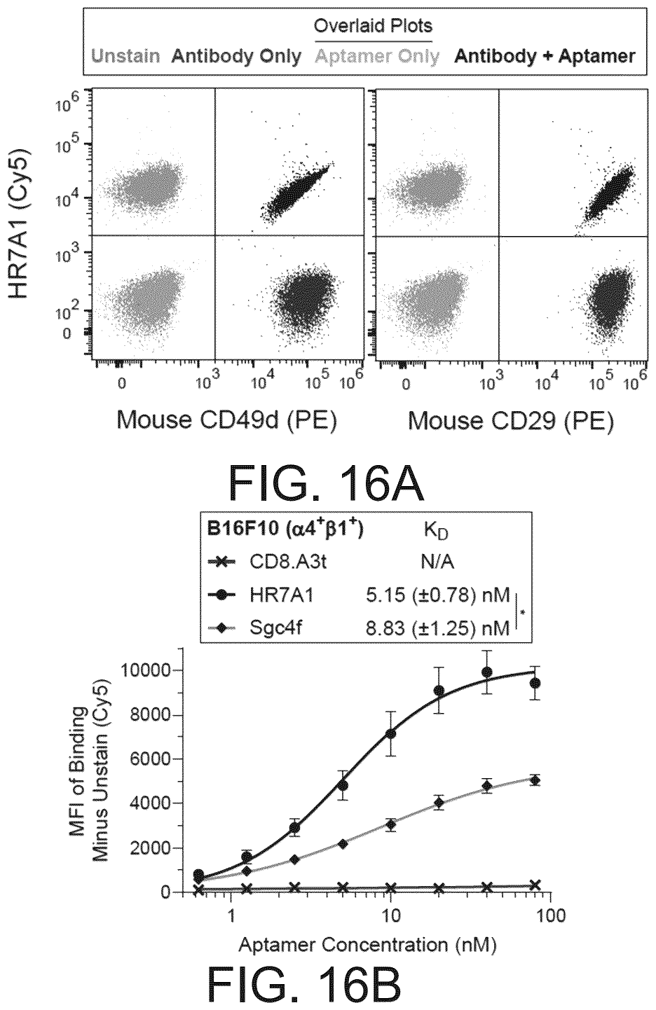

FIGS. 16A, 16B illustrate that HR7A1 binds mouse α4β1. (16A) Overlaid flow cytometry plots of 40 nM Cy5-labeled HR7A1 aptamer co-staining with PE-labeled anti-mouse CD49d and CD29 antibodies on mouse B16F10 cells. Plots are representative of n=1 independent experiment. (16B) Flow cytometry binding curves of Cy5-labeled CD8.A3t, HR7A1, and Sgc4f to B16F10 cells, as determined by MFI. The curves represent a nonlinear regression assuming one-site specific binding with Hill slope. KD values were calculated by averaging the individual regression values of the independent experiments. Data points and error bars, and KD values, represent mean±SD; n=3 independent experiments. *P<0.05 (two-sided unpaired t-test). Cy5, cyanine 5; PE, phycoerythrin.

The domains of the human integrins α4 and β1 that make up the α4β1 headpiece share 88.4% and 92.8% amino acid identity in their extracellular domains with their mouse counterparts, respectively (FIGS. 14, 15). The mouse B16F10 melanoma cell line that is known to express α4β1 (García-Martín et al. (2018) Journal of Cerebral Blood Flow & Metabolism, 39, 1995-2010) was used for evaluation. The full-length HR7A1 displayed high binding to B16F10 cells that correlated strongly with anti-mouse CD49d and anti-mouse CD29 antibody co-staining (Supplementary FIG. 16A). Whereas a control CD8-binding CD8.A3t aptamer showed no binding to B16F10 cells, HR7A1 bound with an apparent KD of 5.2 nM (FIG. 16B), compared to the 2-3 nM apparent KD it displays towards human H9 and Jurkat cells (FIG. 2C). Sgc4f also bound B16F10 cells, although with lower affinity and binding MFI than HR7A1 (FIG. 16B). Collectively, these results demonstrate that HR7A1 and its truncated variants can bind both human and mouse α4β1 and block fibronectin and VCAM-1 binding to the receptor.

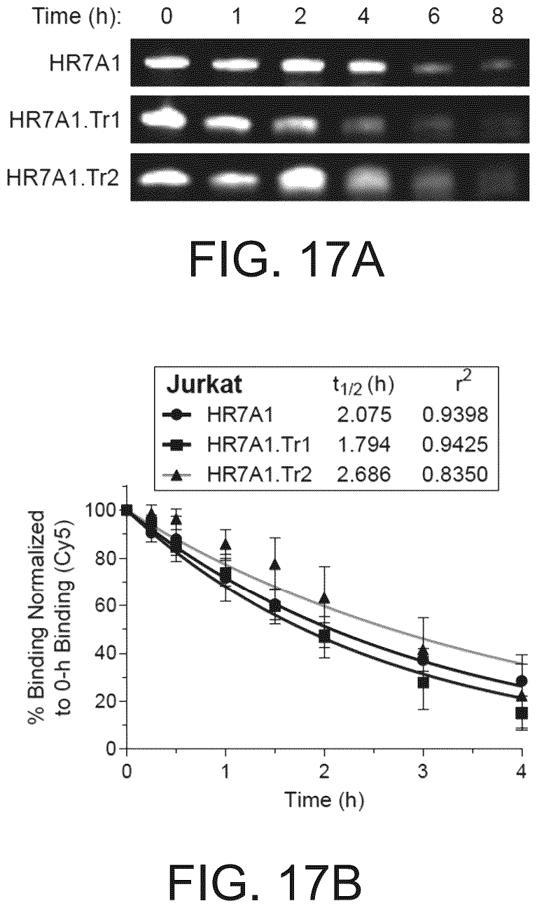

FIGS. 17A, 17B illustrate binding of HR7A1 and truncated variants in serum in this Example. (17A) 15% TBE-urea gel of FAM-labeled HR7A1, HR7A1.Tr1, and HR7A1.Tr2 after 8-h incubation in 50% normal mouse serum at 37° C. Gel image is representative of n=1 independent experiment. (17B) Flow cytometry binding of 10 nM Cy5-labeled HR7A1, HR7A1.Tr1, and HR7A1.Tr2 to Jurkat cells after 4-h incubation in 50% normal mouse serum at 37° C., normalized to a 0-h no incubation control. The curves represent a nonlinear regression assuming one-phase exponential decay. Serum half-life values were calculated from a single regression of the averaged experimental data. Data points and error bars represent mean±SD; n=3-5 independent experiments. FAM, 6-carboxyfluorescein; Cy5, cyanine 5.

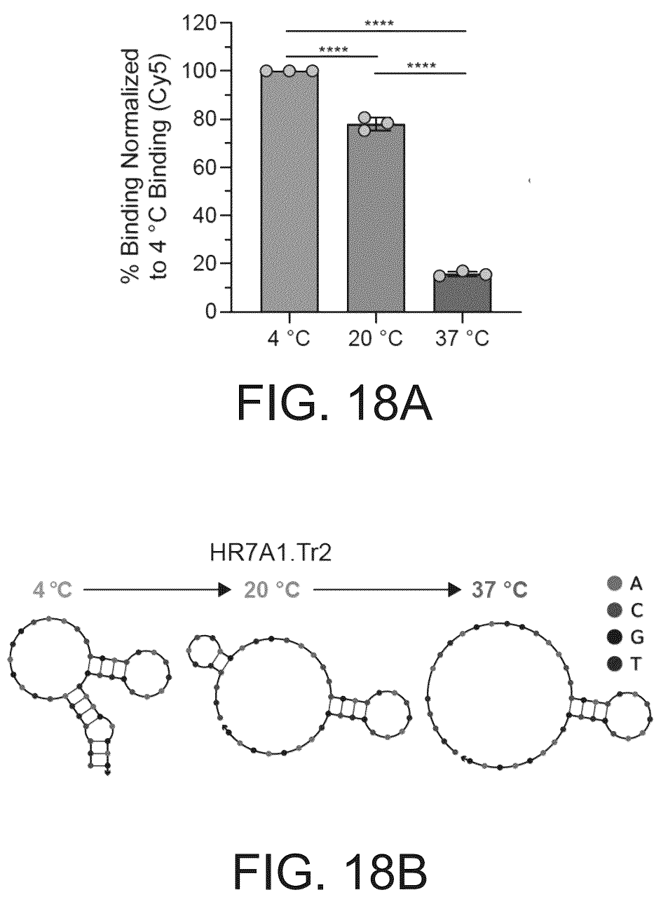

FIGS. 18A, 18B illustrate binding of HR7A1.Tr2 at physiological temperature. (18A) Flow cytometry binding of 5 nM Cy5-labeled HR7A1.Tr2 to Jurkat cells at 4, 20, and 37° C., normalized to binding at 4° C. Non-specific binding of a SCRM control at each temperature was subtracted before normalizing data. Data points and error bars represent mean±SD; n=3 independent experiments. ****P<0.0001 (ordinary one-way ANOVA with Tukey's test). (18B) MFE secondary structure of HR7A1.Tr2 at 4, 20, and 37° C., predicted using NUPACK (Na+=137 mM; Mg2+=5.5 mM). Cy5, cyanine 5.

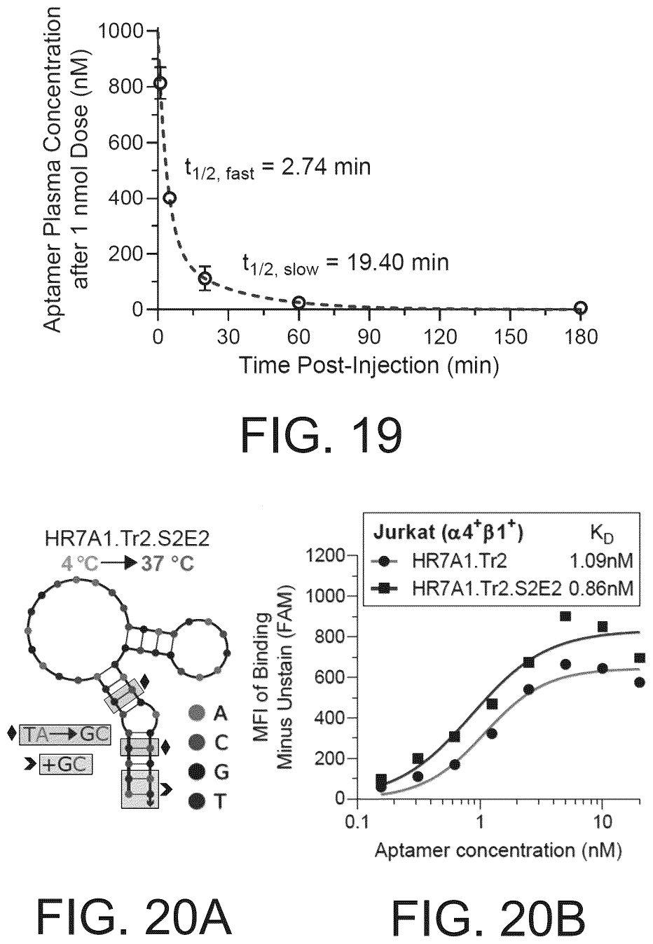

FIG. 19 illustrates circulation half-lives of unmodified aptamers in vivo. Plasma half-life of a 50-nt Cy5-labeled scrambled aptamer (SCRM) in C57BL/6 mice following 1 nmol retro-orbital injection. The curve represents a nonlinear regression assuming two-phase exponential decay. Data points and error bars represent the mean±SD; n=3 mice. Cy5, cyanine 5.

Aptamer-polymer conjugates for overcoming barriers to in vivo translation. Aptamers face many barriers for in vivo usage, including poor serum stability, temperature-sensitive binding, and rapid kidney clearance (White et al., (2000) J Clin Invest, 106, 929-934; Wang et al., (2020) ACS Sens, 5, 3246-3253). Post-SELEX modification of aptamers can address these barriers, allowing translation of aptamers from binding ligands that function solely under controlled in vitro conditions to robust targeting reagents that can actualize a therapeutic effect in vivo (Ni et al., (2017) Int J Mol Sci, 18, 1683). To this end, the impact of serum, temperature, and renal clearance on the functionality of the described aptamers was evaluated to identify areas for improvement. Investigating functional serum stability, it was found that HR7A1, HR7A1.Tr1, and HR7A1.Tr2 exhibit short half-lives of 1.8-2.7 h, respectively, in 50% normal mouse serum (FIGS. 17A, 170B). As these half-lives are expected to be even shorter under more physiologically relevant conditions (i.e., fresh 100% normal mouse serum), serum stability poses a major challenge for translation of these aptamers into mice. Next, for evaluating temperature-sensitive binding, HR7A1.Tr2 was pre-equilibrated at 4, 20 and 37° C. before performing cell binding at these respective temperatures to mimic an in vivo setting where the aptamer would circulate at physiological temperature for some time before encountering target cells. HR7A1.Tr2 retained only 78% and 16% of its 4° C. binding at 20 and 37° C., respectively, identifying another issue for in vivo translation (FIG. 18A). Lastly, to estimate the circulation half-life of the aptamers, 1 nmol of a Cy5-labeled scrambled aptamer (SCRM) was injected into C57BL/6 mice and monitored the aptamer plasma concentration over 3 h. Fitting the exponential decay of aptamer plasma concentration over time to a two-compartment pharmacokinetic model, the scrambled aptamer was cleared rapidly from the blood, displaying a distribution half-life of 3 min and an elimination half-life of 19 min (FIG. 19). Unsurprisingly, these results demonstrate that HR7A1.Tr2 requires additional engineering before it can be feasibly used for cancer targeting in vivo.

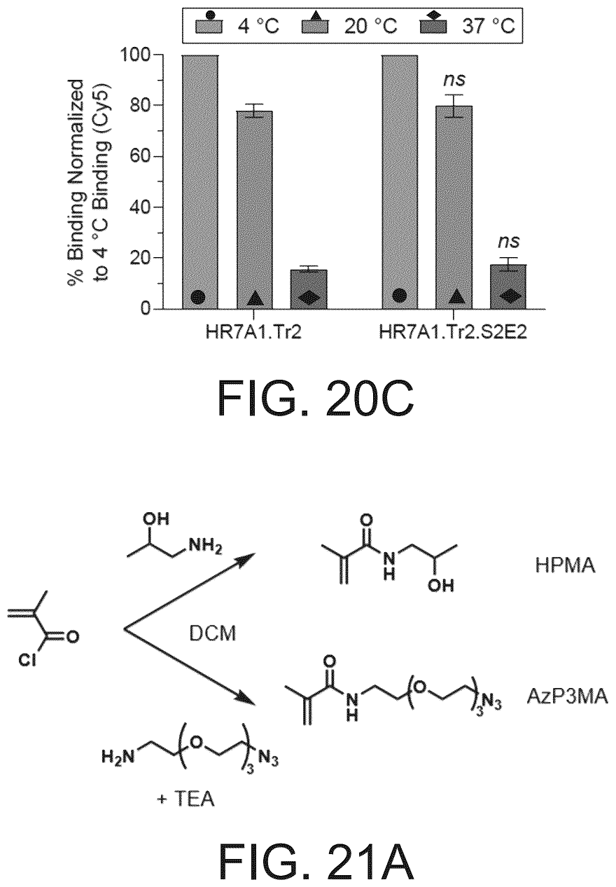

FIGS. 20A-20C illustrate that HR7A1.Tr2.S2E2 preserves binding affinity of HR7A1.Tr2. (20A) MFE secondary structure of HR7A1.Tr2.S2E2 at 4 and 37° C., predicted using NUPACK (Na+=137 mM; Mg2+=5.5 mM). Highlighting denotes base pairs that were substituted into (diamong) or appended to (chevron) the parental HR7A1.Tr2 sequence to create HR7A1.Tr2.S2E2. (20B) Flow cytometry binding curves of FAM-labeled HR7A1.Tr2 and HR7A1.Tr2.S2E2 to Jurkat cells, as determined by MFI. The curves represent a nonlinear regression assuming one-site specific binding with Hill slope. Data points are representative of n=1 independent experiment. (20C) Flow cytometry binding of 5 nM Cy5-labeled HR7A1.Tr2 and HR7A1.Tr2.S2E2 to Jurkat cells at 4, 20, and 37° C., normalized to binding at 4° C. Non-specific binding of a SCRM control at each temperature was subtracted before normalizing data. Data points and error bars represent mean±SD; n=3 independent experiments. ns>0.05 (ordinary two-way ANOVA with Sidak correction). Cy5, cyanine 5.

To stabilize HR7A1.Tr2 for in vivo usage, two adenine-thymine (AT) base pairs were substituted in the HR7A1.Tr2 stem region with more stable GC base pairs and also appended two more GC base pairs at the 5′ and 3′ end of the aptamer, resulting in the HR7A1.Tr2.S2E2 sequence that maintains it predicted MFE structure between 4 and 37° C. (FIG. 20A and Table 1). Encouragingly, HR7A1.Tr2.S2E2 preserved the binding affinity of the parental HR7A1.Tr2 aptamer and even exhibited higher maximal binding (FIG. 20B), indicating that the nucleotide substitutions and additions that were made to the HR7A1.Tr2 stem reversed the fluorescence quenching observed in FIG. 2C.

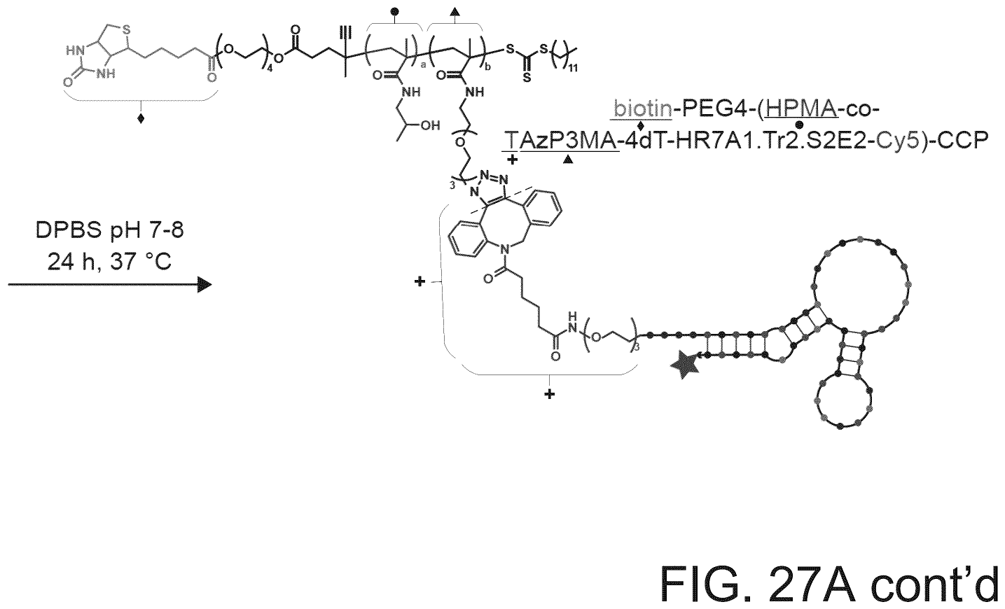

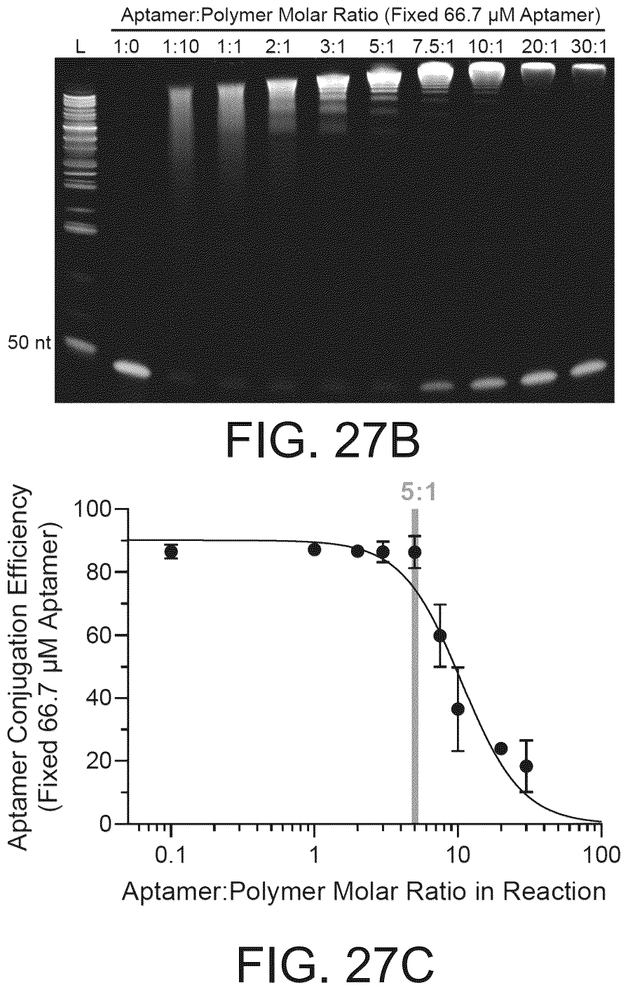

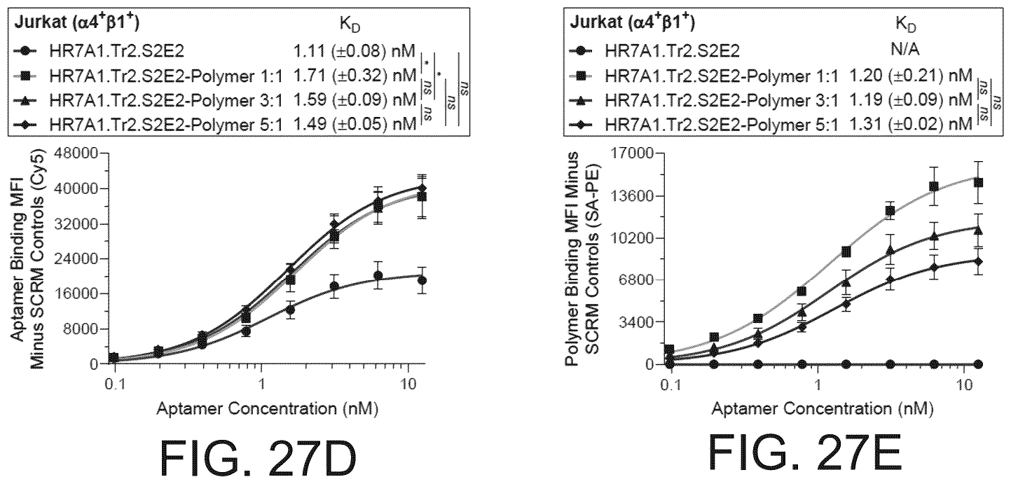

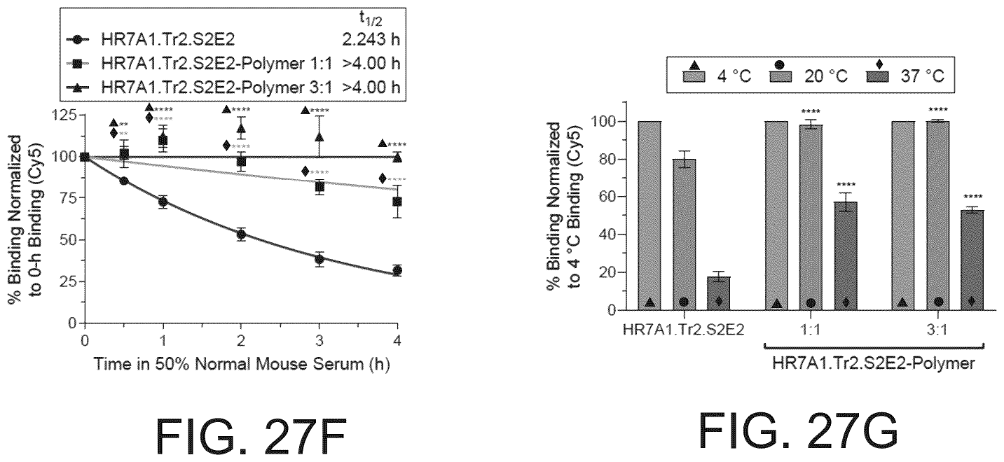

FIGS. 27A-27G illustrate multivalent aptamer-polymer conjugates constructed with metal-free click chemistry address issues with aptamer serum and thermal stability. (27A) Schematic representation of dual DBCO-TEG-and Cy5-labeled HR7A1.Tr2.S2E2 aptamer conjugation to biotinylated HPMA-AzP3MA polymer by strain-promoted azide-alkyne cycloaddition (SPAAC). Biotin, HPMA, and AzP3MA monomers within the polymer structure are shown by a diamond, a circle, and a triangle, respectively, whereas DBCO-TEG and Cy5 modifications on the 5′ and 3′ ends of the aptamer are shown by a cross and a star, respectively. Dashed line indicates separation between HPMA polymer and DBCO-TEG modification. (27B) SYBR Gold-stained 15% urea-PAGE gel of aptamer conjugation to polymer at different molar ratios, with fixed 66.7 μM aptamer in each reaction. The large upward shift in the aptamer band size signifies successful conjugation of aptamer onto the polymer via SPAAC, and further shifts in conjugate band size reflects different aptamer valencies on the polymer. (27C) Semi-quantitative analysis of aptamer conjugation efficiency to the polymer at different molar ratios, as measured by reduction in SYBR gold signal intensity of the unconjugated aptamer band in the urea-PAGE gel. Data point and error bars represent mean±SD; n=2 independent gels ran from the same reactions. (27D and 27E) Flow cytometry binding curves of free HR7A1.Tr2.S2E2 and 1:1, 3:1, and 5:1 HR7A1.Tr2.S2E2-polymer conjugates to Jurkat cells, as determined by MFI of (27D) the Cy5 label on the aptamer and (27E) SA-PE labeling of the biotinylated polymer minus the binding MFI of matched SCRM controls. The curves represent a nonlinear regression assuming one-site specific binding with Hill slope. KD values were calculated by averaging the individual regression values of the independent experiments. Data points and error bars, and KD values, represent mean±SD; n=3 independent experiments. ns>0.05, *P<0.05 (ordinary one-way ANOVA with Tukey's test). (27F) Flow cytometry binding of 5 nM Cy5-labeled free HR7A1.Tr2.S2E2 and 1:1 and 3:1 HR7A1.Tr2.S2E2-polymer conjugates to Jurkat cells after 4-h incubation in 50% normal mouse serum at 37° C., normalized to a 0-h no incubation control. The curves represent a nonlinear regression assuming one-phase exponential decay. Serum half-life values were calculated from a single regression of the averaged experimental data. Data points and error bars represent mean±SD; n=3 independent experiments. **P<0.01, ****P<0.0001 (diamond: significance between HR7A1.Tr2.S2E2 and 1:1 HR7A1.Tr2.S2E2-polymer; triangle: significance between HR7A1.Tr2.S2E2 and 3:1 HR7A1.Tr2.S2E2-polymer; ordinary two-way ANOVA with Dunnett's test). (27G) Flow cytometry binding of 5 nM Cy5-labeled free HR7A1.Tr2.S2E2 and 1:1 and 3:1 HR7A1.Tr2.S2E2-polymer conjugates to Jurkat cells at 4, 20, and 37° C., normalized to binding at 4° C. Non-specific binding of matched SCRM controls at each temperature was subtracted before normalizing data. Data points and error bars represent mean±SD; n=3 independent experiments. ****P<0.0001 (ordinary two-way ANOVA with Dunnett's test). DBCO, dibenzocyclooctyne; TEG, triethylene glycol; PEG, polyethylene glycol; HPMA, 2-hydroxypropyl methacrylamide; AzP3MA; 11-azido-3,6,9-trioxaundecan-1-methacrylamide; CCP, 4-Cyano-4-(dodecylsulfanylthiocarbonyl)sulfanylpentanoic acid; Cy5, cyanine 5; SA-PE, streptavidin phycoerythrin.

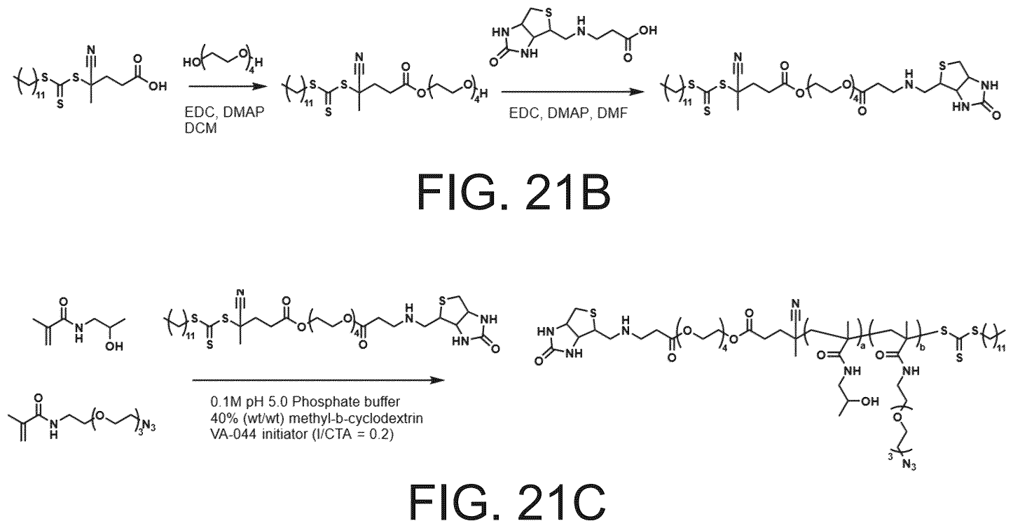

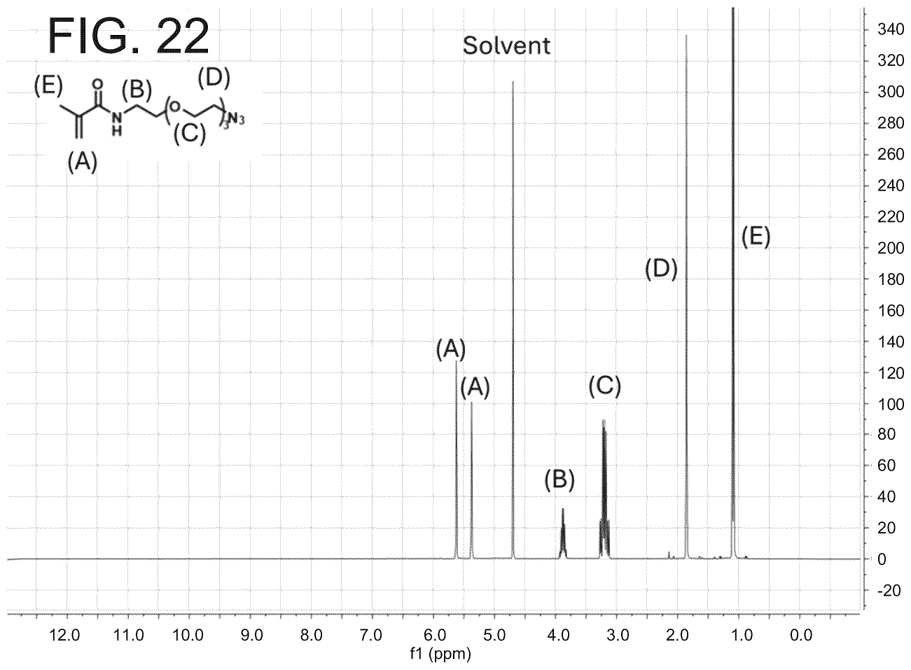

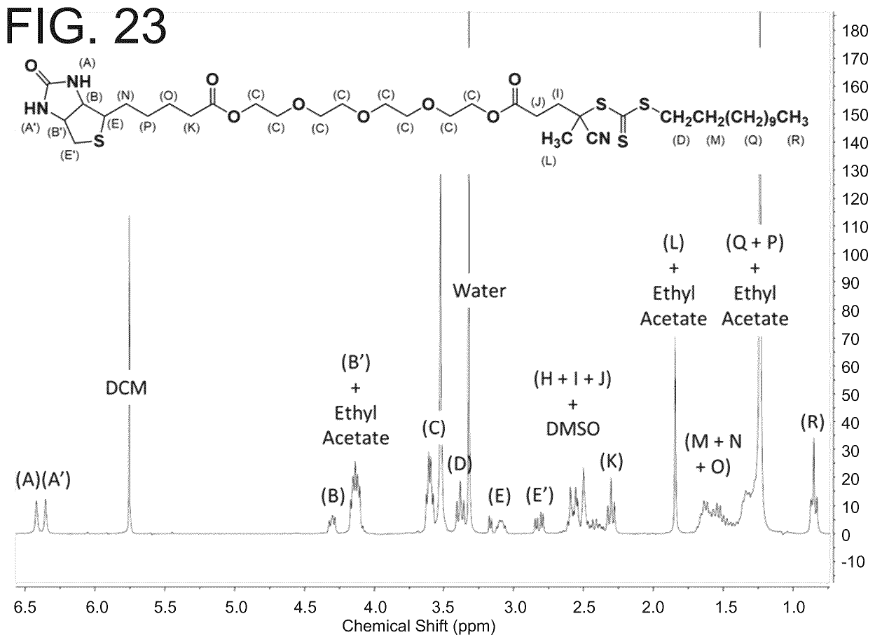

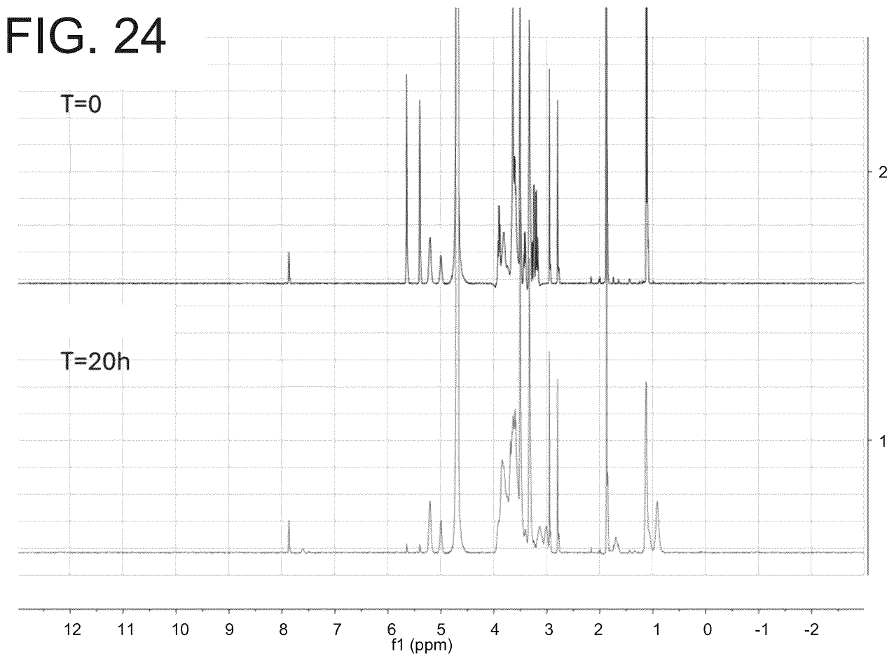

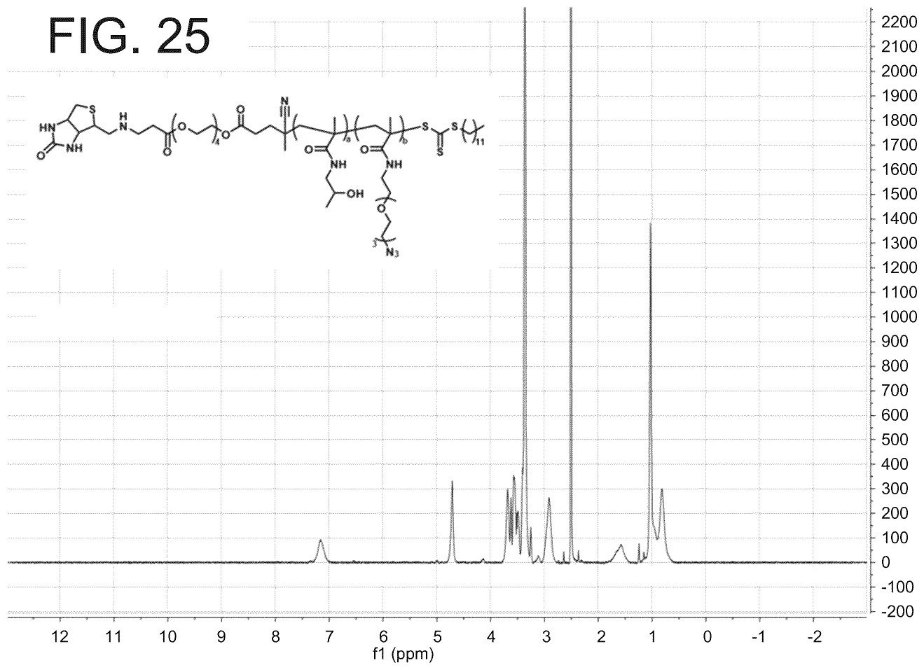

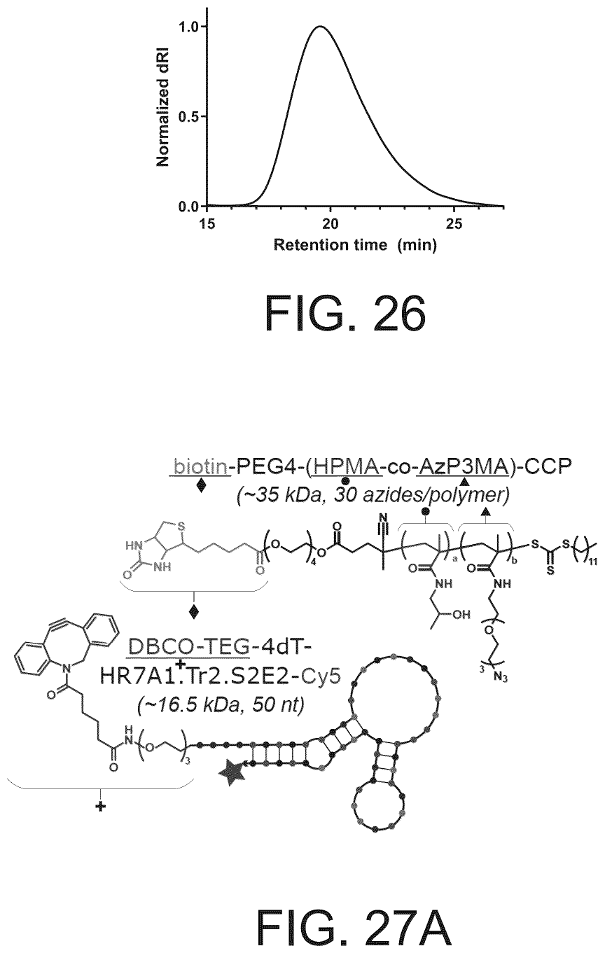

Accordingly, polymeric display of HR7A1.Tr2.S2E2 was next explored for comprehensive improvement of aptamer temperature-sensitive binding, serum stability, and circulation time, as this method has been well characterized in the literature to improve aptamer targeting and biodistribution in vivo (Yang et al., (2011) J Am Chem Soc, 133, 13380-13386; Fletcher et al., (2018) Chemical Communications, 54, 11538-11541; Deng et al., (2020) Bioconjug Chem, 31, 37-42; Nerantzaki et al., (2021) Polym Chem, 12, 3498-3509). Importantly, polymer conjugation is generally considered non-immunogenic and enables multivalent display of aptamer (Yang 2011, supra; Hoang Thi et al., (2020) Polymers (Basel), 12, 298; Ozer et al., (2022) Advanced Materials, 34, 2107852), unlike PEGylation that can cause life-threatening allergic reactions in patients with pre-existing anti-PEG antibodies (Ganson et al., (2016) Journal of Allergy and Clinical Immunology, 137, 1610-1613). For the polymer itself, 2-hydroxypropyl methacrylamide (HPMA) and 11-azido-3,6,9-trioxaundecan-1-methacrylamide (AzP3MA) monomers were first synthesized as well as a biotin-PEG4 4-Cyano-4-(dodecylsulfanylthiocarbonyl)sulfanylpentanoic (biotin-PEG4-CCP) chain transfer reagent (FIGS. 21A, 21B), and proper synthesis was confirmed by 1H NMR (FIGS. 22, 23). These components were copolymerized via reversible addition-fragmentation chain transfer (RAFT) to create the biotin-PEG4-(HPMA-co-AzP3MA)-CCP polymer (FIG. 21C). 1H NMR confirmed successful copolymerization of the monomers (FIGS. 24, 25), and conversion calculations as well as gel permeation chromatography (GPC) estimated a polymer molecular weight of 35-53 kDa with 30 azide groups/polymer (FIG. 26 and Table 8). The azide groups allow conjugation of dibenzocyclooctyne (DBCO) functionalized ligands to the polymer via strain-promoted azide-alkyne cycloaddition (SPAAC), a metal-free click reaction that occurs efficiently under mild aqueous conditions to form a stable triazole (Taz) (Lutz, J.-F. (2008) Angewandte Chemie Intl Ed, 47, 2182-2184). For aptamer attachment to the polymer, a dual-functionalized HR7A1.Tr2.S2E2 was designed with a 5′ DBCO-TEG modification further extended by a four thymidine spacer (4dT) and a 3′ Cy5 label (FIG. 27A).

TABLE 8
Composition information of polymer.

| Polymer | DP/# of | DP/# of | Mn (kDa, | Mn | |
|---|---|---|---|---|---|
| Conversion | HPMA | AzP3MA | NMR, | (kDa, | |
| (%, NMR) | (NMR) | (NMR) | theoretical) | GPC) | PDI |
| 95 | 183 | 30 | 35.3 | 52.8 | 1.35 |

Table 8 lists compositional information of biotin-PEG4-(HPMA-co-AzP3MA)-CCP polymer.

To optimize both the conjugation efficiency and multivalent display of the aptamer on the polymer, small-scale SPAAC reactions were performed with a fixed concentration of the modified aptamer and varying amounts of polymer and assessed conjugation using an electrophoretic mobility shift assay (FIG. 27B). Aptamer conjugation to the polymer resulted in a large upward shift in the aptamer band on urea-PAGE (i.e., less mobile aptamer), and a laddering effect was also observed, meaning a given reaction produced a dispersity of aptamer valencies on the polymer. Importantly, varying the aptamer-to-polymer ratio in the SPAAC reactions provided flexible control over the average aptamer valency on the polymer, as indicated by the shift in the conjugate band at different ratios. Using the disappearance of the unconjugated aptamer band to semi-quantitatively interpret the conjugation efficiency, it was found that a 5:1 aptamer-to-polymer ratio or lower, which translates to at least a 6-fold excess of polymer azide groups over aptamer DBCO groups, maximized aptamer conjugation with 90% efficiency (FIG. 27C). As increasing the ratio further led to a sharp decline in conjugation efficiency, 1:1, 3:1, and 5:1 aptamer-polymer ratios were selected for large-scale SPAAC reactions, purification by molecular weight cut-off membrane filtration, and further testing.

Evaluating binding of the aptamer-polymer conjugates to Jurkat cells by the Cy5 label on the individual aptamers, all three ratios of conjugate display similar aptamer binding affinities to that of the free aptamer with KD values of 1.1-1.7 nM (FIG. 27D), demonstrating that conjugation to the polymer does not negatively affect aptamer binding. Interestingly, the maximal Cy5 binding fluorescence of the aptamer-polymer conjugates is higher than that of the free aptamer, which indicates slower off-kinetics with the conjugates that leads to higher steady-state binding. The maximal Cy5 binding fluorescence for the conjugates does not change regardless of the aptamer-polymer ratio, indicating that most aptamers on each polymer participate in binding. Supporting this, when binding of the conjugates by the biotin label on the polymer was evaluated, decreasing maximal binding fluorescence was seen with increasing aptamer-to-polymer ratio, signifying that it takes less polymer to saturate cell binding when you have higher valencies of the aptamer on the polymer (FIG. 27E). While enhanced apparent binding affinities were not observed with the multivalent aptamer-polymer conjugates that would be expected when avidity is at play, it is important to highlight that these binding curves are graphed by aptamer concentration and not conjugate concentration and binding was only carried out for 30 min. To truly observe avidity enhancements caused by a decreased dissociation rate, a much longer incubation period would be required to reach equilibrium at low concentrations (Vauquelin and Charlton. (2013) Br J Pharmacol, 168, 1771-1785).

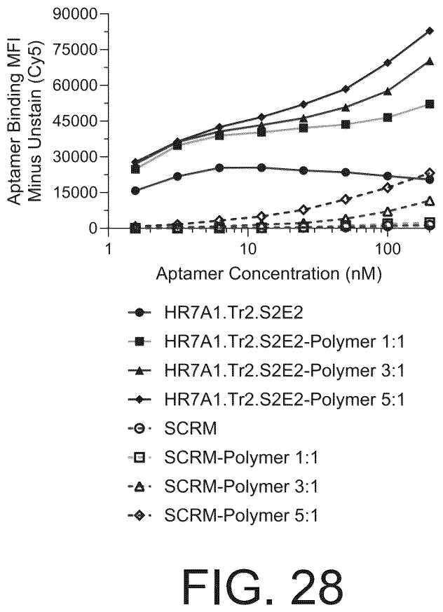

FIG. 28 illustrates that higher valency aptamer-polymer conjugates display more non-specific cell binding. Flow cytometry binding curves of free HR7A1.Tr2.S2E2, 1:1, 3:1, and 5:1 HR7A1.Tr2.S2E2-polymer conjugates, and matched SCRM controls to Jurkat cells, as determined by MFI of the Cy5 label on the aptamer. The curves represent a nonlinear regression assuming one-site specific binding with Hill slope. Data points are representative of n=1 independent experiment. Cy5, cyanine 5.

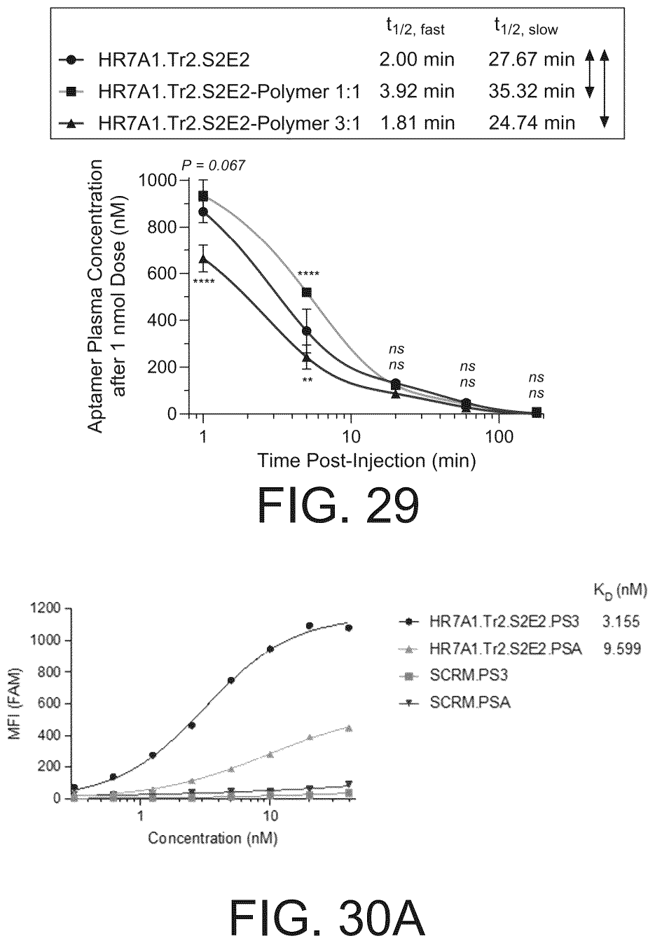

FIG. 29 illustrates that aptamer-polymer conjugates have slightly altered circulation half-lives compared to free aptamers. Plasma half-life of Cy5-labeled free HR7A1.Tr2.S2E2 and 1:1 and 3:1 HR7A1.Tr2.S2E2-polymer conjugates in C57BL/6 mice following 1 nmol retro-orbital injection. The curves represent a nonlinear regression assuming two-phase exponential decay, and the x-axis is plotted on a log-scale to better resolve data from earlier timepoints. Data points and error bars represent the mean±SD; n=3 mice. ns>0.05, **P<0.01, ****P<0.0001 (ordinary two-way ANOVA with Dunnett's test). Cy5, cyanine 5.

As increasing the ratio of aptamer-to-polymer on the conjugates led to increased non-specific binding at high concentrations (FIG. 28), which is consistent with reported observations with aptamer-conjugated nanoparticles (Woythe et al., (2023) Journal of Controlled Release, 355, 228-237), it was elected to only pursue the 1:1 and 3:1 aptamer-polymer conjugates for further assessment of serum stability, temperature sensitive binding, and circulation half-life. Evaluating functional serum stability, the 1:1 and 3:1 aptamer-polymer conjugates displayed markedly prolonged half-lives in 50% normal mouse serum (>4 h) compared to the free aptamer that had a serum half-life of only 2.2 h (FIG. 27F). These findings are consistent with the polymer shielding effect described by others that drives increased aptamer biostability (Deng et al., (2020) Bioconjug Chem, 31, 37-42). Similarly, it was found that the 1:1 and 3:1 aptamer-polymer conjugates also better retained binding at physiological temperatures, retaining 98-100% and 53-57% of their binding at 20 and 37° C., respectively, compared to 80% and 18% for the free aptamer (FIG. 27G). Thus, aptamer conjugation to polymers leads to all-around improvements in serum and thermal stability. The circulation half-life of the aptamer-polymer conjugates in C57BL/6 mice was also assessed, and an improvement in the plasma concentration was observed at early timepoints (1 and 5 min) with the 1:1 aptamer-polymer conjugate compared to the free aptamer (FIG. 29). Of importance, the 3:1 aptamer-polymer conjugate was cleared quicker than the free aptamer, indicating that a higher negative charge-to-mass ratio may negatively influence circulation half-life, potentially due to selective renal uptake of anionic polymers (Liu et al. (2018) Biomaterials, 178, 317-325). Accordingly, decreasing aptamer valency and/or increasing polymer size may lead to improved circulation half-life. Taken together, HR7A1.Tr2.S2E2 conjugation to polymer leads to improved biostability that will support future in vivo cancer targeting applications.

### Experimental Example 2

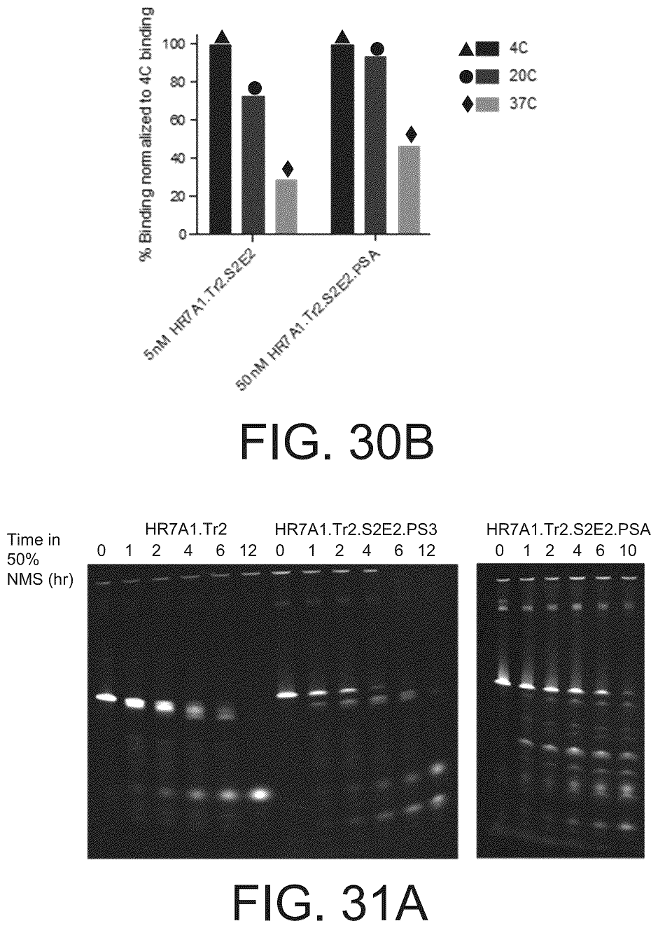

Binding assay: Jurkat cells were washed with DPBS and incubated with Zombie Violet (1:500, 10⁷ cells/mL, BioLegend) in DPBS for live/dead staining for 15 min at room temperature. After live/dead staining, cells were washed with aptamer washed buffer supplemented with 1% BSA to quench the remaining dye and aliquoted in a 96-well plate at 1-2e5 cells/well. For aptamer staining, cells were incubated with annealed aptamers at the indicated concentrations in 100 μL binding buffer (aptamer wash buffer supplemented with 1% BSA and 0.1 mg/ml yeast RNA) for 20-30 min at 4° C. For binding analysis at different temperatures, aptamers (diluted in binding buffer) and cells were separately incubated at the indicated temperatures for 20 min for equilibration. The equilibrated aptamer solutions were then added to the cells and allowed to incubate for 20-30 min. After incubation, cells were washed 2 times with 200 μL aptamer wash buffer supplemented with 1% BSA, resuspended in 200 μL aptamer wash buffer supplemented with 1% BSA and 0.2% PFA, and analyzed on an Attune NxT cytometer. FIG. 30A illustrates that phosphorothioate-substituted HR7A1.Tr2.S2E2 aptamers PS3 and PSA retain binding to Jurkat cells, with apparent KD values of 3.2 and 9.6 nM, respectively. FIG. 30B illustrates that the phosphorothioate-substituted HR7A1.Tr2.S2E2.PSA aptamer displays improved thermal stability than the parental HR7A1.Tr2.S2E2 aptamer, retaining more binding to Jurkat cells at both 20° C. and 37° C. relative to its 4° C. binding.

TABLE 9
Aptamer sequences.

| | Sequence |
|---|---|
| Name | (* denotes a phosphorothioate bond) |
| HR7A1.Tr2. | C*G*C*GCCGTACTAGATGCAACCCGACTACTAACGTC |
| S2E2.PS3 | GTACGAGC*G*C*G |
| | (SEQ ID NO: 66) |
| HR7A1.Tr2. | C*G*CGCCGTA*CTA*GA*TGCA*A*CCCGA CTA |
| S2E2.PSA | CTA A*CGTCGTA CGA GCG*C*G |
| | (SEQ ID NO: 67) |

Table 9 lists the aptamer sequences used in this Example.

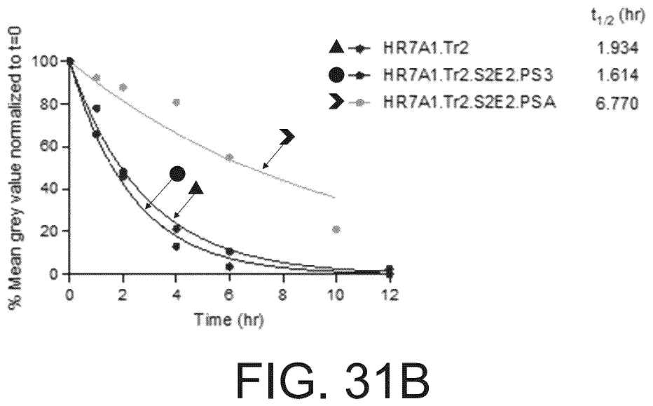

Serum stability analysis: FAM-labeled aptamers were annealed at 6 μM in aptamer wash buffer by heating at 95° C. for 5 min followed by snap cooling on ice. Annealed aptamers were incubated with equal volumes of 100% normal mouse serum at 37° C. for 10-12 hours. Samples of the aptamer serum mixtures were taken at the indicated time points and frozen at −80 C. To visualize aptamer degradation in serum, aptamer serum mixtures taken at different time points were analyzed by urea-PAGE and band intensities were quantified by ImageJ analysis. FIGS. 31A and 31B illustrate that the more phosphorothioate-substituted HR7A1.Tr2.S2E2.PSA aptamer displays improved serum stability than the parental HR7A1.Tr2.S2E2 aptamer and the less phosphorothioate-substituted HR7A1.Tr2.S2E2.PS3 aptamer, displaying a serum half-life of 6.8 h compared <2 h for the other aptamers.

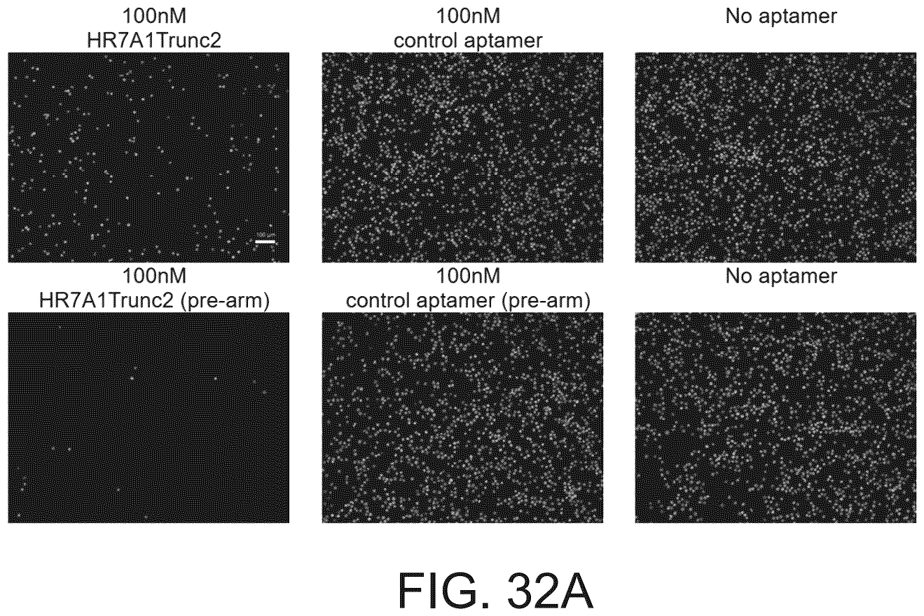

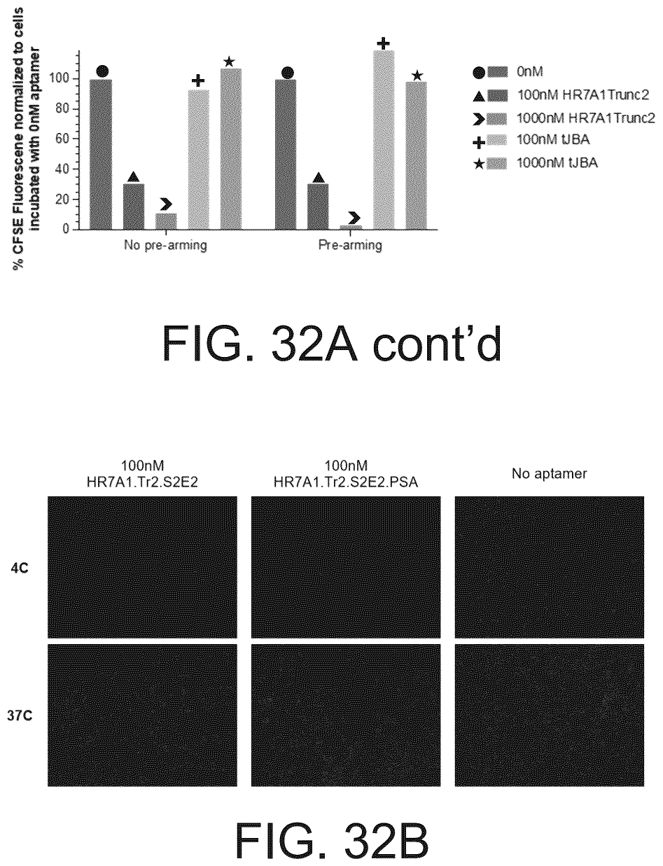

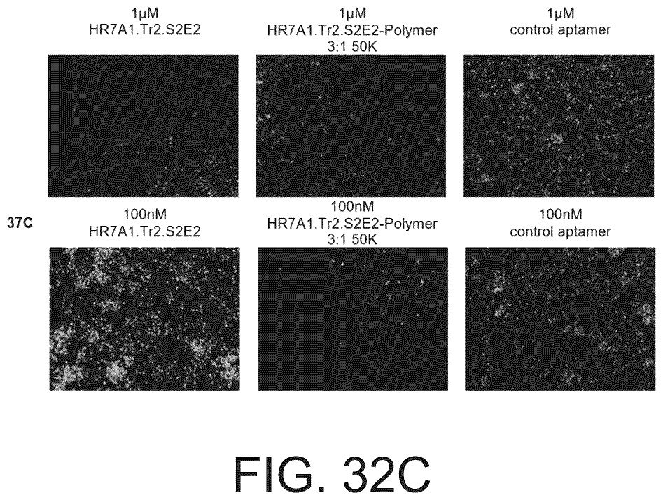

Leukocyte adhesion assay: Each well of a NUNC™ MaxiSorp plate was coated with 100 nM His tagged VCAM-1 protein in 100 μL DPBS overnight at 4° C. The plate was washed 3 times with 200 μL DPBS 1% BSA to remove non-adsorbed proteins and was further blocked with 200 μL DPBS 1% BSA for 1 hr at room temperature. For fluorescence imaging and quantification, Jurkat cells were labeled with 1 μM CellTrace CFSE or 0.5 μM CellTrace Far Red at 1e6 cells/mL in DPBS for 20 min at RT, followed by a 1:3 dilution with RPMI 10% FBS to inactivate remaining dyes. For aptamer competition with pre-arming, labeled cells were first incubated with aptamers or controls in 100 μL binding buffer (aptamer wash buffer supplemented with 1% BSA and 0.1 mg/mL yeast tRNA for 30 min at 4° C. The pre-armed cells were then applied to the VCAM-1-coated wells and allowed to adhere for 30 min-1 hour at 4° C. or 37° C. For aptamer competition without pre-arming, labeled Jurkat cells were resuspended in binding buffer with aptamers and directly applied to the VCAM-1-coated plate for adhesion. The plate was washed 3 times with aptamer wash buffer 1% BSA and analyzed on a fluorescence microscope or a microplate reader. FIG. 32A illustrates that 100 nM and 1 μM HR7A1.Tr2 can inhibit Jurkat cell adhesion to VCAM-1 coated plates at 4° C. by >70% and >90%, respectively, regardless of the cells being pre-armed with aptamer or not. FIG. 32B illustrates that 100 nM HR7A1.Tr2.S2E2 and phosphorothioate-substituted HR7A1.Tr2.S2E2.PSA inhibit Jurkat cell adhesion to VCAM-1 coated plates less at 37° C. compared to 4° C. FIG. 32C illustrates that the 3:1 HR7A1.Tr2.S2E2-polymer conjugate displays improved inhibition of Jurkat cell adhesion to VCAM-1 coated plates at 37° C. compared to free aptamer, especially at lower concentrations.

### Example Clauses

1. An oligonucleotide that specifically binds α4β1, the oligonucleotide including a sequence having at least 75% sequence identity to SEQ ID NO: 65.

2. The oligonucleotide of clause 1, wherein the oligonucleotide has a length of 22 to 150 nucleotides.

3. The oligonucleotide of clause 1 or 2, wherein the oligonucleotide has a length of 35 to 129 nucleotides.

4. The oligonucleotide of at least one of clauses 1-3, wherein the oligonucleotide has:
at least 80%, at least 95%, at least 97%, at least 98%, or at least 99% sequence identity to the sequence set forth as SEQ ID NO: 1, SEQ ID NO: 5, SEQ ID NO: 6, SEQ ID NO: 8, or SEQ ID NO: 10; or
the sequence set forth in SEQ ID NO: 1, SEQ ID NO: 5, SEQ ID NO: 6, SEQ ID NO: 8, or SEQ ID NO: 10.

5. The oligonucleotide of at least one of clauses 1-4, wherein an affinity between the oligonucleotide and α4β1 is less than 1 μM.

6. The oligonucleotide of at least one of clauses 1-5, wherein an affinity between the oligonucleotide and the α4β1 is less than 100 nM.

7. The oligonucleotide of at least one of clauses 1-6, wherein the oligonucleotide is conjugated to a polymer, a solid support, a tag, a linker, a protein, or a lipid, or
wherein the oligonucleotide further includes a phosphorothioate bond.

8. The oligonucleotide of clause 7, wherein the polymer includes a water-soluble polymer and/or poly[N-(2-Hydroxypropyl) methacrylamide-11-azido-3,6,9-trioxaundecan-1-methacrylamide] (poly[HPMA-AzP3MA]),
wherein the solid support includes a magnetic bead, paper, glass, or a polymer,
wherein the tag includes a fluorophore, biotin, or a dye, or
wherein the lipid is within a cell membrane, a liposome, a lipid nanoparticle, a microbubble, or an extracellular vesicle.

9. A composition including the oligonucleotide of at least one of clauses 1 to 8 and a pharmaceutically acceptable carrier.

10. The composition of clause 9, further including a polymer or a dendrimer, wherein the polymer or the dendrimer is conjugated to the oligonucleotide.

11. The composition of at least one of clauses 9 or 10, wherein the polymer includes poly[N-(2-Hydroxypropyl) methacrylamide-11-azido-3,6,9-trioxaundecan-1-methacrylamide] (poly[HPMA-AzP3MA)]).

12. A method including:
administering a therapeutically effective dosage of the composition including:
an oligonucleotide that specifically binds α4β1, the oligonucleotide including a sequence having at least 75% sequence identity to SEQ ID NO: 65; and
a pharmaceutically acceptable carrier to a subject.

13. The method of clause 12, wherein the subject has a disease, and the therapeutically effective dosage treats the subject, the disease including sickle cell disease, cancer, an autoimmune disease, autoimmune encephalomyelitis multiple sclerosis (MS), rheumatoid arthritis (RA), inflammatory bowel syndrome (IBS), a T-cell mediated autoimmune disease, duchenne muscular dystrophy, dry eye disease, or dry age-related macular degeneration.

14. The method of clause 12 or 13, wherein the composition further includes a polymer or a dendrimer, wherein the polymer or the dendrimer is conjugated to the oligonucleotide, and/or
wherein the oligonucleotide of clause 1 further includes a phosphorothioate bond.

15. The method of at least one of clauses 12-14, further including:
administering a therapeutically effective dosage of a chemotherapy to the subject; and/or
administering a therapeutically effective dosage of an immunotherapy to the subject.

16. The method of clause 15, wherein the chemotherapy includes a cytotoxic chemotherapy.

17. The method of clause 16, wherein the cytotoxic chemotherapy includes cytarabine; nilotinib; vincristine, dexamethasone, L-asparaginase (VDL); doxorubicin; paclitaxel; or an auristatin.

18. The method of at least one of clauses 15-17, wherein the immunotherapy includes a checkpoint inhibitor, a bispecific antibody, a bispecific T-cell engager, an adoptive cell therapy, a cancer vaccine, or a cytokine.

19. A method of isolating cells that express α4β1, the method including:
obtaining a solution including a mixture of cells;
exposing the solution to an oligonucleotide conjugated to a solid support, the oligonucleotide including a sequence having at least 75% sequence identity to SEQ ID NO: 65; and
removing the solid support from the exposed solution, thereby isolating cells that express α4β1.

20. The method of clause 19, further including:
releasing the cells that express α4β1 from the oligonucleotide by:
administering a reversal construct to the isolated cells;
changing a pH of the solution;
changing a salt concentration of the solution;
changing a chelator concentration of the solution;
changing a temperature of the solution;
applying a physical force to the solution; or
administering a nuclease enzyme to the solution.

21. A method of isolating cells that express α4β1, the method including:
obtaining a solution including a mixture of cells;
exposing the solution to an oligonucleotide of any one of clauses 1 to 16 bound to a magnetic bead; and
applying a magnetic field to the exposed solution, thereby isolating cells that express α4β1.

22. The method of clause 21, wherein the cells that express α4β1 include lymphocytes.

23. The method of clause 21 or 22, further including:
administering a reversal construct to the isolated cells, thereby releasing the cells that express α4β1 from the oligonucleotide.

24. A method of transforming stem cells into immune cells, the method including:
exposing the stem cells to the oligonucleotide of claim 1 conjugated to a microbead, thereby transforming the stem cells into the immune cells.

25. The method of clause 24, wherein the stem cells include induced pluripotent stem cells (iPSCs), and/or
wherein the immune cells include T cells.

26. A method of detecting α4β1-expressing cells, the method including:
exposing cells within a subject or within a biological sample derived from the subject to the oligonucleotide of claim 1, wherein the oligonucleotide is conjugated to a label configured to output a detection signal; and
detecting the detection signal,
thereby detecting the α4β1-expressing cells.

27 The method of clause 26, wherein the subject has a disease, and
wherein the detecting detects the disease.

28. The method of clause 26 or 27, wherein the label includes a radionuclide, a contrast agent, or a fluorescent tag,
wherein the detection signal includes one or more photons, or
wherein detecting the detection signal includes imaging the cells.

29 The method of clause 28, wherein the contrast agent includes gadolinium, and
wherein imaging the cells includes performing magnetic resonance imaging (MRI).

30. A polymer conjugated to at least two oligonucleotides of claim 1.

31. The polymer of clause 30, wherein an oligonucleotide-to-polymer ratio is in a range of about 1:1 to about 10:1,
wherein the polymer has a molecular weight in a range of about 20 to about 100 kDa, or
wherein the polymer is bound to a second polymer.

32. The polymer of clause 31, wherein the second polymer changes chemical or physical properties in response to a stimulus, the stimulus including a temperature, a pH, photomodulation, or a chemical reduction, and/or
wherein the second polymer includes poly(N-isopropylacrylamide).

33. The polymer of at least one of clauses 30-32, further including poly[N-(2-Hydroxypropyl) methacrylamide-11-azido-3,6,9-trioxaundecan-1-methacrylamide] (poly[HPMA-AzP3MA]).

34. The polymer of at least one of clauses 30-33, further including a water-soluble polymer.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (±3.0); Lys (±3.0); aspartate (±3.0+1); glutamate (+3.0+1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding domain (of, for example, a CAR binding domain or a nanoparticle selected cell targeting ligand) to its cognate binding molecule with an affinity or Ka (i.e., an equilibrium association constant of a particular binding interaction with units of 1/M) equal to or greater than 10⁵ M⁻¹, while not significantly associating with any other molecules or components in a relevant environment sample. “Specifically binds” is also referred to as “binds” herein. Binding domains may be classified as “high affinity” or “low affinity”. In particular embodiments, “high affinity” binding domains refer to those binding domains with a Ka of at least 107 M-1, at least 108 M-1, at least 109 M-1, at least 1010 M-1, at least 1011 M-1, at least 1012 M-1, or at least 1013 M-1. In particular embodiments, “low affinity” binding domains refer to those binding domains with a Ka of up to 107 M-1, up to 106 M-1, up to 105 M-1. Alternatively, affinity may be defined as an equilibrium dissociation constant (Kd) of a particular binding interaction with units of M (e.g., 10-5 M to 10-13 M). In certain embodiments, a binding domain may have “enhanced affinity,” which refers to a selected or engineered binding domains with stronger binding to a cognate binding molecule than a wild type (or parent) binding domain. For example, enhanced affinity may be due to a Ka (equilibrium association constant) for the cognate binding molecule that is higher than the reference binding domain or due to a Kd (dissociation constant) for the cognate binding molecule that is less than that of the reference binding domain, or due to an off-rate (Koff) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

1. An oligonucleotide that specifically binds α4β1, the oligonucleotide comprising a sequence having at least 75% sequence identity to SEQ ID NO: 65.

2. The oligonucleotide of claim 1, wherein the oligonucleotide has a length of 22 to 150 nucleotides.

3. The oligonucleotide of claim 1, wherein the oligonucleotide has:
at least 80%, at least 95%, at least 97%, at least 98%, or at least 99% sequence identity to the sequence set forth as SEQ ID NO: 1, SEQ ID NO: 5, SEQ ID NO: 6, SEQ ID NO: 8, or SEQ ID NO: 10; or
the sequence set forth in SEQ ID NO: 1, SEQ ID NO: 5, SEQ ID NO: 6, SEQ ID NO: 8, or SEQ ID NO: 10.

4. The oligonucleotide of claim 1, wherein an affinity between the oligonucleotide and α4β1 is less than 1 μM.

5. The oligonucleotide of claim 1, wherein the oligonucleotide is conjugated to a polymer, a solid support, a tag, a linker, a protein, or a lipid, or
wherein the oligonucleotide further comprises a phosphorothioate bond.

6. The oligonucleotide of claim 5, wherein the polymer comprises a water-soluble polymer and/or poly[N-(2-Hydroxypropyl) methacrylamide-11-azido-3,6,9-trioxaundecan-1-methacrylamide] (poly[HPMA-AzP3MA]),
wherein the solid support comprises a magnetic bead, paper, glass, or a polymer,
wherein the tag comprises a fluorophore, biotin, or a dye, or
wherein the lipid is within a cell membrane, a liposome, a lipid nanoparticle, a microbubble, or an extracellular vesicle.

7. A method comprising:
administering a therapeutically effective dosage of a composition comprising:
an oligonucleotide that specifically binds α4β1, the oligonucleotide comprising a sequence having at least 75% sequence identity to SEQ ID NO: 65; and
a pharmaceutically acceptable carrier to a subject.

8. The method of claim 7, wherein the subject has a disease, and the therapeutically effective dosage treats the subject, the disease comprising sickle cell disease, cancer, an autoimmune disease, autoimmune encephalomyelitis multiple sclerosis (MS), rheumatoid arthritis (RA), inflammatory bowel syndrome (IBS), a T-cell mediated autoimmune disease, duchenne muscular dystrophy, dry eye disease, or dry age-related macular degeneration.

9. The method of claim 7, wherein the composition further comprises a polymer or a dendrimer, wherein the polymer or the dendrimer is conjugated to the oligonucleotide, and/or
wherein the oligonucleotide of claim 1 further comprises a phosphorothioate bond.

10. The method of claim 7, further comprising:
administering a therapeutically effective dosage of a chemotherapy to the subject; and/or
administering a therapeutically effective dosage of an immunotherapy to the subject.

11. A method of isolating cells that express α4β1, the method comprising:
obtaining a solution comprising a mixture of cells;
exposing the solution to an oligonucleotide conjugated to a solid support, the oligonucleotide comprising a sequence having at least 75% sequence identity to SEQ ID NO: 65; and
removing the solid support from the exposed solution, thereby isolating cells that express α4β1.

12. The method of claim 11, further comprising:
releasing the cells that express α4β1 from the oligonucleotide by:
administering a reversal construct to the isolated cells;
changing a pH of the solution;
changing a salt concentration of the solution;
changing a chelator concentration of the solution;
changing a temperature of the solution;
applying a physical force to the solution; or
administering a nuclease enzyme to the solution.

13. A method of transforming stem cells into immune cells, the method comprising:
exposing the stem cells to the oligonucleotide of claim 1 conjugated to a microbead, thereby transforming the stem cells into the immune cells.

14. The method of claim 13, wherein the stem cells comprise induced pluripotent stem cells (iPSCs), and/or
wherein the immune cells comprise T cells.

15. A method of detecting α4β1-expressing cells, the method comprising:
exposing cells within a subject or within a biological sample derived from the subject to the oligonucleotide of claim 1, wherein the oligonucleotide is conjugated to a label configured to output a detection signal; and
detecting the detection signal,
thereby detecting the α4β1-expressing cells.

16. The method of claim 15, wherein the subject has a disease, and
wherein the detecting detects the disease.

17. The method of claim 15, wherein the label comprises a radionuclide, a contrast agent, or a fluorescent tag,
wherein the detection signal comprises one or more photons, or
wherein detecting the detection signal comprises imaging the cells.

18. A polymer conjugated to at least two oligonucleotides of claim 1.

19. The polymer of claim 18, wherein an oligonucleotide-to-polymer ratio is in a range of about 2:1 to about 10:1,
wherein the polymer has a molecular weight in a range of about 20 to about 100 kDa, or
wherein the polymer is bound to a second polymer.

20. The polymer of claim 19, wherein the second polymer changes chemical or physical properties in response to a stimulus, the stimulus comprising a temperature, a pH, photomodulation, or a chemical reduction, and/or
wherein the second polymer comprises poly(N-isopropylacrylamide).


SEQUENCE LISTING (71 sequences)

SEQ ID NO: 1 - DNA, 88 bp, synthetic construct
  source 1..88: mol_type = other DNA
atccagagtgacgcagcaaacctgacctccttactagatgcaacccgactactaacgtcgtaagagagcctggacacggtggcttagt

SEQ ID NO: 2 - DNA, 81 bp, synthetic construct
  source 1..81: mol_type = other DNA
atccagagtgacgcagcaaattccaaactcgagtaagcgtagagcctctcatcgcctcaataatggacacggtggcttagt

SEQ ID NO: 3 - DNA, 51 bp, synthetic construct
  source 1..51: mol_type = other DNA
gcagcagcgtaaagggggtgtttgtgcggtgtggagtgcgcgtgctgctgc

SEQ ID NO: 4 - DNA, 88 bp, synthetic construct
  source 1..88: mol_type = other DNA
atccagagtgacgcagcaacagaggtgtagaagtacacgtgaacaagcttgaaattgtctctgacagaggtggacacggtggcttagt

SEQ ID NO: 5 - DNA, 55 bp, synthetic construct
  source 1..55: mol_type = other DNA
cagcaaacctgacctccttactagatgcaacccgactactaacgtcgtaagagag

SEQ ID NO: 6 - DNA, 41 bp, synthetic construct
  source 1..41: mol_type = other DNA
tccttactagatgcaacccgactactaacgtcgtaagagag

SEQ ID NO: 7 - DNA, 27 bp, synthetic construct
  source 1..27: mol_type = other DNA
ctagatgcaacccgactactaacgtcg

SEQ ID NO: 8 - DNA, 37 bp, synthetic construct
  source 1..37: mol_type = other DNA
gcgtactagatgcaacccgactactaacgtcgtacgc

SEQ ID NO: 9 - DNA, 56 bp, synthetic construct
  source 1..56: mol_type = other DNA
atcacttataacgagtgcggatgcaaacgccagacagggggacaggagataagtga

SEQ ID NO: 10 - DNA, 46 bp, synthetic construct
  source 1..46: mol_type = other DNA
cgcgccgtactagatgcaacccgactactaacgtcgtacgagcgcg

SEQ ID NO: 11 - DNA, 59 bp, synthetic construct
  source 1..59: mol_type = other DNA
aatgatacggcgaccaccgagatctacaccgaggagataccactaagccaccgtgtcca

SEQ ID NO: 12 - DNA, 61 bp, synthetic construct
  source 1..61: mol_type = other DNA
caagcagaagacggcatacgagatgaatggacacagaccgtcgatccagagtgacgcagca

SEQ ID NO: 13 - DNA, 52 bp, synthetic construct
  source 1..52: mol_type = other DNA
aacctgacctccttactagatgcaacccgactactaacgtcgtaagagagcc

SEQ ID NO: 14 - DNA, 52 bp, synthetic construct
  source 1..52: mol_type = other DNA
tagtcaaggcgatagacgaaaaacattgttttcctggaaggcccagaatctg

SEQ ID NO: 15 - DNA, 52 bp, synthetic construct
  source 1..52: mol_type = other DNA
cgcagcagcgtaaagggggtgtttgtgcggtgtggagtgcgcgtgctgctgc

SEQ ID NO: 16 - DNA, 52 bp, synthetic construct
  source 1..52: mol_type = other DNA
tagtcaaggcgatagacgaaaaacattgttttcctggaaggcccggaatctg

SEQ ID NO: 17 - DNA, 52 bp, synthetic construct
  source 1..52: mol_type = other DNA
ggttgctgtacctacaattgacaataaaaaatctagcgggcttagagacagt

SEQ ID NO: 18 - DNA, 52 bp, synthetic construct
  source 1..52: mol_type = other DNA
aggcgctagacgcaaacacttaagcaccatgagtgccaagttttcctaatgg

SEQ ID NO: 19 - DNA, 52 bp, synthetic construct
  source 1..52: mol_type = other DNA
ataagtacgcagtcaaggcgatagacgaaaagaacactttcccggaagggcc

SEQ ID NO: 20 - DNA, 52 bp, synthetic construct
  source 1..52: mol_type = other DNA
atagtcaaggcgatagacgaaaaacattgttttcctggaaggcccagaatcg

SEQ ID NO: 21 - DNA, 52 bp, synthetic construct
  source 1..52: mol_type = other DNA
gcggaaccgaagatttggccaaaaaaatcaatcgggctttggctcaccccgt

SEQ ID NO: 22 - DNA, 52 bp, synthetic construct
  source 1..52: mol_type = other DNA
gatgcaaatgctcgagagaggcacggcgcaaatctggtagatgagcaacgta

SEQ ID NO: 23 - DNA, 52 bp, synthetic construct
  source 1..52: mol_type = other DNA
agcctgacctccttactagatgcaacccgactactaacgtcgtaagagagcc

SEQ ID NO: 24 - DNA, 52 bp, synthetic construct
  source 1..52: mol_type = other DNA
aacctgacctccttactagatgcaacccgactactaacgtcgtaagagagtc

SEQ ID NO: 25 - DNA, 52 bp, synthetic construct
  source 1..52: mol_type = other DNA
aacctgacctccttactagatgcagcccgactactaacgtcgtaagagagcc

SEQ ID NO: 26 - DNA, 52 bp, synthetic construct
  source 1..52: mol_type = other DNA
gcgcaagtatggactatcccaacaaggcgttagacgtaaatgaattatttcc

SEQ ID NO: 27 - DNA, 52 bp, synthetic construct
  source 1..52: mol_type = other DNA
tcacatctcggcagatgcaaatgcctggccaggaacccgaaacctgtgatag

SEQ ID NO: 28 - DNA, 52 bp, synthetic construct
  source 1..52: mol_type = other DNA
cgcagcaacccaaaccaccagccggggatgcaaacaccgcacagggaacggc

SEQ ID NO: 29 - DNA, 52 bp, synthetic construct
  source 1..52: mol_type = other DNA
agctgtaggtgtgcggttaactccgtgctagataggaggtatctccacaggg

SEQ ID NO: 30 - DNA, 52 bp, synthetic construct
  source 1..52: mol_type = other DNA
cgcagcaatcacagggctacaaggtgctaaacgtaaactagcaagagaacta

SEQ ID NO: 31 - DNA, 52 bp, synthetic construct
  source 1..52: mol_type = other DNA
gagtaggaagagtaaccaacaaggcggtagacgtaaaaagatcaattttccg

SEQ ID NO: 32 - DNA, 52 bp, synthetic construct
  source 1..52: mol_type = other DNA
gacgttgattaggcggatgcaaacgcacggtcgtggtaccaaaacgcactgc

SEQ ID NO: 33 - DNA, 52 bp, synthetic construct
  source 1..52: mol_type = other DNA
gaagagagtccggatgcaaacgcctgactaggatagacgatggttctgtgta

SEQ ID NO: 34 - DNA, 52 bp, synthetic construct
  source 1..52: mol_type = other DNA
aggtgctaaacgcaaacctacctctgctgagggcaaggtgtttcctacggtg

SEQ ID NO: 35 - DNA, 52 bp, synthetic construct
  source 1..52: mol_type = other DNA
acgcagcaagcggttttcgggttcgggtctgggggttgggttgtcggcacta

SEQ ID NO: 36 - DNA, 52 bp, synthetic construct
  source 1..52: mol_type = other DNA
gtgacacgttccaaatccaccaacatcggatgcaaacgccagacatgggtaa

SEQ ID NO: 37 - DNA, 52 bp, synthetic construct
  source 1..52: mol_type = other DNA
aacctgacctccttactagatgcaacccgactactaacgtcgtaagagatcc

SEQ ID NO: 38 - DNA, 52 bp, synthetic construct
  source 1..52: mol_type = other DNA
cgcagcagcttaaagggggtgtttgtgcggtgtggagtgcgcgtgctgctgc

SEQ ID NO: 39 - DNA, 52 bp, synthetic construct
  source 1..52: mol_type = other DNA
aacctgacctccttgctagatgcaacccgactactaacgtcgtaagagagcc

SEQ ID NO: 40 - DNA, 52 bp, synthetic construct
  source 1..52: mol_type = other DNA
aagctttattggggatgcaaacaccgaacagggaacaatagattccagagca

SEQ ID NO: 41 - DNA, 52 bp, synthetic construct
  source 1..52: mol_type = other DNA
aacctgacctccttactagatgcaacccgactgctaacgtcgtaagagagcc

SEQ ID NO: 42 - DNA, 52 bp, synthetic construct
  source 1..52: mol_type = other DNA
cggccccaagcatcaaggcgttagacgaaacccagggttcacgtagtgtaat

SEQ ID NO: 43 - DNA, 52 bp, synthetic construct
  source 1..52: mol_type = other DNA
cgacgtggttgacggatgcaaacgcctgacaagggaatcaatagtggtgtaa

SEQ ID NO: 44 - DNA, 52 bp, synthetic construct
  source 1..52: mol_type = other DNA
ttccagacaggggggatgcaaacacatgataatgggaccatggttggaaatc

SEQ ID NO: 45 - DNA, 52 bp, synthetic construct
  source 1..52: mol_type = other DNA
aacccgacctccttactagatgcaacccgactactaacgtcgtaagagagcc

SEQ ID NO: 46 - DNA, 52 bp, synthetic construct
  source 1..52: mol_type = other DNA
aacctgacctccttactagatgcatcccgactactaacgtcgtaagagagcc

SEQ ID NO: 47 - DNA, 52 bp, synthetic construct
  source 1..52: mol_type = other DNA
gatgcaaatgcctgacgagggtgacagcgaaactttggtatataagcacctc

SEQ ID NO: 48 - DNA, 52 bp, synthetic construct
  source 1..52: mol_type = other DNA
gcaagatgcaactgatgactatctaggtggaagctaagtttcaccgtaatgc

SEQ ID NO: 49 - DNA, 52 bp, synthetic construct
  source 1..52: mol_type = other DNA
ttatcggggatgcaaccaacgaatgtggtacgggaatgtctgatgctacaaa

SEQ ID NO: 50 - DNA, 52 bp, synthetic construct
  source 1..52: mol_type = other DNA
agaaatgagcaacggatgcaaacgcccaaacggggaactgcgtatattttct

SEQ ID NO: 51 - DNA, 52 bp, synthetic construct
  source 1..52: mol_type = other DNA
ttgcgtgaagggagatgcaactaacgatagtggtacctgcacccgcactgca

SEQ ID NO: 52 - DNA, 52 bp, synthetic construct
  source 1..52: mol_type = other DNA
cgcagcagcgtaaagggggtgtttgtgcggtgtggagtgcgcttgctgctgc

SEQ ID NO: 53 - DNA, 52 bp, synthetic construct
  source 1..52: mol_type = other DNA
ccatcttgacttcggatgcaaacgcttgaccaagggaaagacatgatgcgct

SEQ ID NO: 54 - DNA, 52 bp, synthetic construct; identical to SEQ ID NO: 53
  source 1..52: mol_type = other DNA

SEQ ID NO: 55 - DNA, 52 bp, synthetic construct
  source 1..52: mol_type = other DNA
gagaatcggggatgcaaccagctacgcggtacggttcacaacttgtacacac

SEQ ID NO: 56 - DNA, 52 bp, synthetic construct
  source 1..52: mol_type = other DNA
tagtcaaggcgataggcgaaaaacattgttttcctggaaggcccagaatctg

SEQ ID NO: 57 - DNA, 52 bp, synthetic construct
  source 1..52: mol_type = other DNA
aacctgacctccttactagatgcaccccgactactaacgtcgtaagagagcc

SEQ ID NO: 58 - DNA, 52 bp, synthetic construct
  source 1..52: mol_type = other DNA
tagtcaaggcgatagacgaaaaacattgttttcctggaaggcccataatctg

SEQ ID NO: 59 - DNA, 52 bp, synthetic construct
  source 1..52: mol_type = other DNA
aaaaacggccaagtcatcaaggcgatagacgaaaacgaatccctgatttccg

SEQ ID NO: 60 - DNA, 52 bp, synthetic construct
  source 1..52: mol_type = other DNA
agaacaaggtgttaaacgttatgccccagcataccatcgaagaggtcggcaa

SEQ ID NO: 61 - DNA, 52 bp, synthetic construct
  source 1..52: mol_type = other DNA
acaggtcaaggcgctagacgaaggaaatctcctccatacggcaatgttgatt

SEQ ID NO: 62 - DNA, 52 bp, synthetic construct
  source 1..52: mol_type = other DNA
caaaagagatggatgcaaacacctgaaaagggcatgtctgagaagtgatata

SEQ ID NO: 63 - RNA, 25 nt, synthetic construct
  source 1..25: mol_type = other RNA
  modified_base 24: mod_base = OTHER; note = thymine
actcttgtcagctaaggtcacattg

SEQ ID NO: 64 - RNA, 27 nt, synthetic construct
  source 1..27: mol_type = other RNA
caatgtgaccttagctgacaagagtaa

SEQ ID NO: 65 - DNA, 29 bp, synthetic construct
  source 1..29: mol_type = other DNA
actagatgcaacccgactactaacgtcgt

SEQ ID NO: 66 - DNA, 46 bp, synthetic construct; identical to SEQ ID NO: 10
  source 1..46: mol_type = other DNA
  modified_base 1: mod_base = OTHER; note = phosphorothioate bond between positions 1 and 2
  modified_base 2: mod_base = OTHER; note = phosphorothioate bond between positions 2 and 3
  modified_base 3: mod_base = OTHER; note = phosphorothioate bond between positions 3 and 4
  modified_base 43: mod_base = OTHER; note = phosphorothioate bond between positions 43 and 44
  modified_base 44: mod_base = OTHER; note = phosphorothioate bond between positions 44 and 45
  modified_base 45: mod_base = OTHER; note = phosphorothioate bond between positions 45 and 46

SEQ ID NO: 67 - DNA, 46 bp, synthetic construct; identical to SEQ ID NO: 10
  source 1..46: mol_type = other DNA
  modified_base 1: mod_base = OTHER; note = phosphorothioate bond between positions 1 and 2
  modified_base 2: mod_base = OTHER; note = phosphorothioate bond between positions 2 and 3
  modified_base 9: mod_base = OTHER; note = phosphorothioate bond between positions 9 and 10
  modified_base 12: mod_base = OTHER; note = phosphorothioate bond between positions 12 and 13
  modified_base 14: mod_base = OTHER; note = phosphorothioate bond between positions 14 and 15
  modified_base 17: mod_base = OTHER; note = phosphorothioate bond between positions 17 and 18
  modified_base 18: mod_base = OTHER; note = phosphorothioate bond between positions 18 and 19
  modified_base 24: mod_base = OTHER; note = phosphorothioate bond between positions 24 and 25
  modified_base 27: mod_base = OTHER; note = phosphorothioate bond between positions 27 and 28
  modified_base 30: mod_base = OTHER; note = phosphorothioate bond between positions 30 and 31
  modified_base 31: mod_base = OTHER; note = phosphorothioate bond between positions 31 and 32
  modified_base 38: mod_base = OTHER; note = phosphorothioate bond between positions 38 and 39
  modified_base 41: mod_base = OTHER; note = phosphorothioate bond between positions 41 and 42
  modified_base 44: mod_base = OTHER; note = phosphorothioate bond between positions 44 and 45
  modified_base 45: mod_base = OTHER; note = phosphorothioate bond between positions 45 and 46

SEQ ID NO: 68 - protein, 432 aa, Homo sapiens
YNVDTESALLYQGPHNTLFGYSVVLHSHGANRWLLVGAPTANWLANASVINPGAIYRCRIGKNPGQTCEQLQLGSPNGEPCGKTCLEERDNQWLGVTLSRQPGENGSIVTCGHRWKNIFYIKNENKLPTGGCYGVPPDLRTELSKRIAPCYQDYVKKFGENFASCQAGISSFYTKDLIVMGAPGSSYWTGSLFVYNITTNKYKAFLDKQNQVKFGSYLGYSVGAGHFRSQHTTEVVGGAPQHEQIGKAYIFSIDEKELNILHEMKGKKLGSYFGASVCAVDLNADGFSDLLVGAPMQSTIREEGRVFVYINSGSGAVMNAMETNLVGSDKYAARFGESIVNLGDIDNDGFEDVAIGAPQEDDLQGAIYIYNGRADGISSTFSQRIEGLQISKSLSMFGQSISGQIDADNNGYVDVAVGAFRSDSAVLLRTRP

SEQ ID NO: 69 - protein, 432 aa, Mus musculus
YNLDPENALLYQGPSGTLFGYSVVLHSHGSKRWLIVGAPTASWLSNASVVNPGAIYRCGIRKNPNQTCEQLQLGSPSGEPCGKTCLEERDNQWLGVTLSRQPGENGSIVTCGHRWKNIFYMKSDNKLPTGICYVMPSDLRTELSKRMAPCYKDYTRKFGENFASCQAGISSFYTQDLIVMGAPGSSYWTGTVFVYNITTNQYKAFVDRQNQVKFGSYLGYSVGAGHFRSPHTTEVVGGAPQHEQIGKAYIFSIDENELNIVYEMKGKKLGSYFGASVCAVDLNADGFSDLLVGAPMQSTIREEGRVFVYINSGMGAVMVEMERVLVGSDKYAARFGESIANLGDIDNDGFEDIAIGAPQEDDLRGAVYIYNGRVDGISSTYSQRIEGQQISKSLRMFGQSISGQIDADNNGYVDVAIGAFHSDSAVLLRTRP

SEQ ID NO: 70 - protein, 445 aa, Homo sapiens
QTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECE

SEQ ID NO: 71 - protein, 445 aa, Mus musculus
QTDKNRCLKANAKSCGECIQAGPNCGWCTNTTFLQEGMPTSARCDDLEALKKKGCQPSDIENPRGSQTIKKNKNVTNRSKGMAEKLRPEDITQIQPQQLLLKLRSGEPQKFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTDRGEFFNELVGQQRISGNLDSPEGGFDAIMQVAVCGSLIGWRNVTRLLVFSTDAGFHFAGDGKLGGIVLPNDGQCHLENNVYTMSHYYDYPSIAHLVQKLSENNIQTIFAVTEEFQPVYKELKNLIPKSAVGTLSGNSSNVIQLIIDAYNSLSSEVILENSKLPDGVTINYKSYCKNGVNGTGENGRKCSNISIGDEVQFEISITANKCPNKESETIKIKPLGFTEEVEVVLQFICKCN